(19) United States
(12) Patent Application Publication
(10) Pub. No.: US 2022/0056044 A1
(43) Pub. Date: Feb. 24, 2022

(54) TRICYCLIC COMPOUNDS AS STING AGONISTS, AND PREPARATION METHODS AND MEDICINAL USES THEREOF

(71) Applicants: Jiangsu Hengrui Medicine Co., Ltd., Lianyungang (CN); Shanghai Hengrui Pharmaceutical Co., Ltd., Shanghai (CN)

(72) Inventors: Dong Liu, Basking Ridge, NJ (US); Puhui Li, Plainsboro, NJ (US); Linghang Zhuang, Chalfont, PA (US); Fengqi Zhang, Edison, NJ (US); Lei Chen, Basking Ridge, NJ (US); Xinzhu Zhang, Franklin Park, NJ (US); Chunying Song, Newtown, PA (US); Suxing Liu, Edison, NJ (US); Matthew Miller, Branchburg, NJ (US); Qiyue Hu, Shanghai (CN); Yuna Yan, Shanghai (CN); Jing Li, Skillman, NJ (US); Feng He, Shanghai (CN); Weikang Tao, North Brunswick, NJ (US)

(21) Appl. No.: 17/413,287

(22) PCT Filed: Dec. 14, 2019
(86) PCT No.: PCT/US19/66413

§ 371 Date: Jun. 11, 2021

Related U.S. Application Data
(60) Provisional application No. 62/779,907, filed on Dec. 14, 2018.

(51) Int. Cl. C07D 498/06; C07D 487/06

(52) U.S. Cl. CPC C07D 498/06; C07D 487/06

(57) ABSTRACT

Compounds of formula (I) useful as agonists of stimulator of interferon genes (STING), the preparation method therefor, pharmaceutical compositions comprising the compounds, and the pharmaceutical uses for the treatment of STING-mediated diseases or disorders are disclosed.

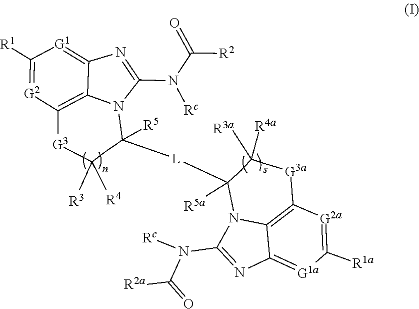

### CROSS-REFERENCE TO RELATED APPLICATIONS

This application claims priority under 35 U.S.C. § 119(e) to U.S. Provisional Patent Application No. 62/779,907, filed on Dec. 14, 2018, the disclosure of which is incorporated herein by reference in its entirety.

### FIELD OF THE INVENTION

The present invention belongs to the filed of medicine and in particular relates to new tricyclic compounds as agonists of stimulator of interferon genes (STING) useful for the treatment of STING-mediated diseases or disorders, and preparation methods thereof.

### BACKGROUND OF THE INVENTION

Vertebrates defend against microorganisms or respond to signals from cellular or tissue damage by innate and adaptive immunity. Innate immunity has no antigen specificity and executes the defense mechanisms immediately after an antigen's appearance in the body. Adaptive immunity requires time to generate a full response, but it is antigen-specific and long lasting. Once an antigen has been processed and recognized, the adaptive immune system utilizes a set of immune cells specifically designed to attack that antigen. During the course of an adaptive immune response, memory immune cells are generated which allow for a more rapid and effective response to re-exposure to antigens. The innate immune system is required to activate our adaptive immune system. Numerous molecules and cells involved in innate immunity and adaptive immunity function cooperatively (Shanker A. and Marincola F., Cancer Immunol. Immunother., 2011, 60: 1061-1074).

Innate immunity is initiated when the pathogen-associated molecular patterns (PAMPs) present in pathogens are recognized by pattern recognition receptors (PRRs) (Medzhitov, R. J. Immunol. 2013, 191, 4473-4474). Some endogenous damage-associated molecular patterns (DAMPs), including various tumor-derived antigens can also be recognized by these PRRs as well (Matzinger, P., Science 2002, 296: 301-305). The free cytosolic DNA from pathogens and abnormal cells can be recognized by DNA sensors. cGAS (cyclic GMP-AMP Synthase) has been shown to be an important DNA sensor and catalyzes free cytosolic DNA into cyclic di-nucleotides (CDN) 2′3′-GAMP (Ng K W., et al, Trends in Immunology, 2018, 39: 44-54).

Stimulator of interferon genes (STING; also known as MITA and MPYS, and encoded by TMEM173) is a signaling molecule associated with the endoplasmic reticulum (ER). Upon binding to the cyclic dinucleotides (CDNs) generated by cGAS as well as bacterial cyclic di-AMP(c-di-AMP) or c-di-GMP in the cytosol, STING undergoes a conformational change and forms a complex with TBK1. This complex translocates from the ER to the perinuclear Golgi and then phosphorylates IRF3, which dimerizes and enters the nucleus to initiate the transcription of type I interferon (IFN)s. TBK1 also phosphorylates residues on the protein IκB, leading to its degradation, which causes the activation and translocation of NF-κB to the nucleus and the transcription of pro-inflammatory cytokines such as TNFα, IL-6 and IL-10 (Ahn J. and Barber G., Current Opinion in Immunology 2014, 31:121-126). Accumulating evidence indicates that STING-dependent signaling is critical in promoting antitumor immunity. STING deficient mice have decreased tumor rejection observed when compared with wild type mice (Woo S. et al, Immunity, 2014, 41:830-842). Activation of STING significantly suppressed the growth of multiple types of mouse tumors (Corrales et al., Cell Reports, 2015, 11:1018-1030).

The antitumor activity mediated by STING is at least partially via type I IFNs (IFNα/β) (Corrales L. and Gajewski F., Clin. Cancer Res., 2015, 21: 4774-4779). The effect of type I IFNs on immune cells has been well established. Upon binding to IFNα/β, the IFNα/β receptor activates a cascade of events and induces the transcription of a wide variety of genes regulated by IFN-stimulated response elements (ISRE), thus modulating multiple types of immune cells. In particular, type I IFNs promote cross-priming, boost effector T cell function and expansion, mediate memory development, thereby coupling innate immunity with adaptive immunity (Zitvogel L. et al, Nature Reviews Immunology, 2015, 15: 405-414). Type I IFNs contribute to antitumor immunity in various types of cancer (Parker B. et al., Nat Rev Cancer, 2016, 16:131-144). TNFα may be another important contributor to the therapeutic effect observed with the activation of STING (Francica B. et al., Cancer Immunol Res., 2018, 6: 1-12).

In summary, the antitumor function of STING signaling has been well-established. The compounds of this invention stimulate the function of STING and accordingly may have a beneficial impact on cancer therapy.

### SUMMARY OF THE INVENTION

The present invention, in one aspect, provides a compound of formula (I), or a pharmaceutically acceptable salt, solvate, or prodrug thereof, including tautomers, cis- or trans-isomers, mesomers, racemates, enantiomers, diastereomers, and mixtures thereof:

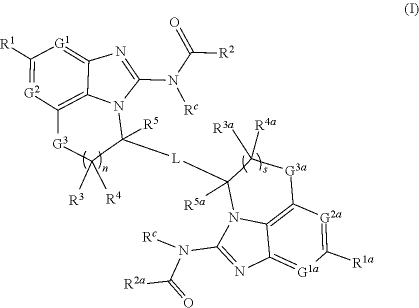

wherein:

G¹, G², G^{1a} and G^{2a} are identical or different, and each is independently N or CR⁶;

G³ and G^{3a} are identical or different, and each is independently O, NR^{g}, or CR⁷R⁸;

L is selected from the group consisting of alkylene, alkenylene, alkynylene, alkylene-Q-alkylene, alkylene-O-alkylene, alkylene-NH-alkylene, alkylene-S(O)ₘ-alkylene, alkylene-C(O)-alkylene, alkylene-C(O)NH-alkylene, alkylene-NHC(O)-alkylene, and alkylene-HNC(O)NH-alkylene, wherein the alkylene, alkenylene, and alkynylene each is unsubstituted or substituted with one or more substituents selected from the group consisting of halogen, alkyl, alkoxy, haloalkyl, amino, nitro, hydroxy, hydroxyalkyl, cyano, cycloalkyl, heterocyclyl, aryl, and heteroaryl;

R^{c} is selected from the group consisting of hydrogen, alkyl, haloalkyl, alkenyl, and alkynyl;

R^{g} is selected from the group consisting of hydrogen, alkyl, cycloalkyl, and alkenyl; wherein the alkyl, cycloalkyl or alkenyl is unsubstituted or substituted with one or more, sometimes preferably one to five, sometimes more preferably one to three, substituents selected from the group consisting of halogen, alkyl, alkoxy, haloalkyl, amino, cyano, hydroxy, hydroxyalkyl, cycloalkyl, heterocyclyl, aryl, and heteroaryl;

R¹ and R^{1a} are identical or different, and each is independently selected from the group consisting of —C(O)NR⁹R¹⁰, —C(O)OR^{m}, hydrogen, halogen, alkyl, alkoxy, haloalkyl, amino, nitro, hydroxy, hydroxyalkyl, cyano, cycloalkyl, heterocyclyl, aryl, and heteroaryl, wherein the alkyl, alkoxy, cycloalkyl, heterocyclyl, aryl or heteroaryl is unsubstituted or substituted with one or more, sometimes preferably one to five, sometimes more preferably one to three, substituents selected from the group consisting of halogen, alkyl, alkoxy, haloalkyl, amino, nitro, hydroxy, hydroxyalkyl, cyano, cycloalkyl, heterocyclyl, aryl, and heteroaryl;

R² and R^{2a} are identical or different, and each is independently selected from the group consisting of alkyl, haloalkyl, cycloalkyl, heterocyclyl, aryl, and heteroaryl; wherein the alkyl, cycloalkyl, heterocyclyl, aryl, or heteroaryl is unsubstituted or substituted with one or more, sometimes preferably one to five, sometimes more preferably one to three, substituents selected from the group consisting of halogen, alkyl, alkoxy, haloalkyl, amino, nitro, cyano, hydroxy, hydroxyalkyl, cycloalkyl, heterocyclyl, aryl, and heteroaryl;

R³, R⁴, R^{3a} and R^{4a} are identical or different, and each is independently selected from the group consisting of hydrogen, halogen, alkyl, alkoxy, haloalkyl, amino, nitro, hydroxy, hydroxyalkyl, cyano, cycloalkyl, heterocyclyl, aryl, and heteroaryl;

R⁵ and R^{5a} are identical or different, and each is independently selected from the group consisting of hydrogen, halogen, alkyl, alkoxy, haloalkyl, amino, nitro, hydroxy, hydroxyalkyl, and cyano;

R⁶ is selected from the group consisting of hydrogen, halogen, alkyl, alkoxy, haloalkyl, amino, nitro, hydroxy, hydroxyalkyl, cyano, cycloalkyl, heterocyclyl, aryl, and heteroaryl;

R⁷ and R⁸ are identical or different, and each is independently selected from the group consisting of hydrogen, halogen, alkyl, alkoxy, haloalkyl, amino, nitro, hydroxy, hydroxyalkyl, cyano, cycloalkyl, heterocyclyl, aryl, and heteroaryl;

R⁹ and R¹⁰ are identical or different, and each is independently selected from the group consisting of hydrogen, alkyl, haloalkyl, hydroxy, cycloalkyl, heterocyclyl, aryl, and heteroaryl; wherein the alkyl, cycloalkyl, heterocyclyl, aryl, or heteroaryl is unsubstituted or substituted with one or more, sometimes preferably one to five, sometimes more preferably one to three, substituents selected from the group consisting of halogen, alkyl, alkoxy, haloalkyl, amino, nitro, cyano, hydroxy, hydroxyalkyl, cycloalkyl, heterocyclyl, aryl, and heteroaryl;

R^{m} is selected from the group consisting of hydrogen, alkyl, haloalkyl, hydroxyalkyl, cycloalkyl, heterocyclyl, aryl, and heteroaryl;

Q is selected from the group consisting of cycloalkyl, heterocyclyl, aryl, and heteroaryl;

m is 0, 1 or 2;

n is 0, 1, 2 or 3; and

s is 0, 1, 2 or 3.

In another aspect, the present invention provides a pharmaceutical composition, comprising a compound of formula (I), or a pharmaceutically acceptable salt, solvate, or prodrug thereof, and a pharmaceutically acceptable carrier.

In another aspect, the present invention provides a method for treating a STING-mediated disease or disorder, comprising administering to a subject in need thereof a therapeutically effective amount of a compound of formula (I), or a pharmaceutically acceptable salt, solvate, or prodrug thereof, or a pharmaceutical composition comprising a compound of formula (I), or a pharmaceutically acceptable salt, solvate, or prodrug thereof.

In another aspect, the present invention relates to use of a compound of formula (I), or a pharmaceutically acceptable salt, solvate, or prodrug thereof, in the manufacture of a medicament for treatment of a STING-mediated disease or disorder, wherein the disease or disorder is selected from a cancer, a pre-cancerous syndrome, and viral infections, preferably a cancer and a pre-cancerous syndrome.

In another aspect, this invention provides a method for preparing a compound of formula (I), or a pharmaceutically acceptable salt, solvate, or prodrug thereof, the method comprising a step of reacting a compound of formula (IA) with a compound of formula (IB) to obtain the compound of formula (I):

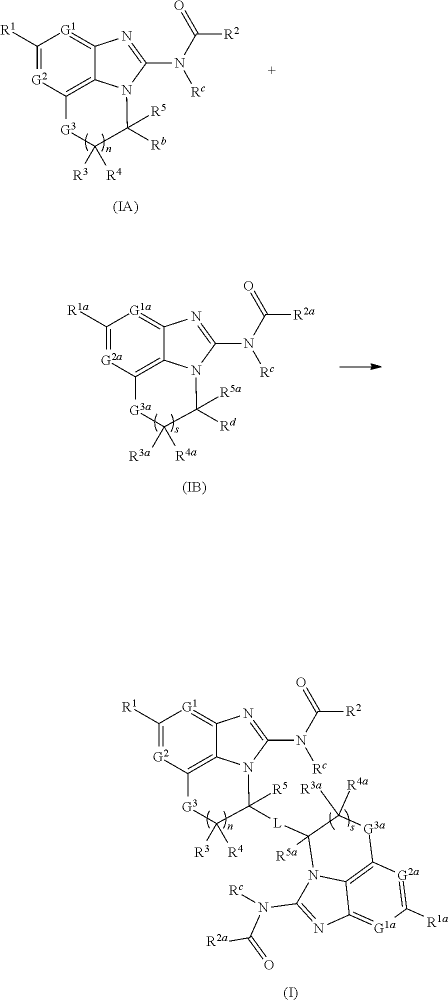

wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3; and

R¹ to R⁵, R^{1a} to R^{5a}, R^{c}, G¹ to G³, G^{1a} to G^{3a}, n and s are each as defined in formula (I).

In another aspect, this invention provides a method for preparing a compound of formula (IG), or a pharmaceutically acceptable salt, solvate, or prodrug thereof, the method comprising a step of reacting a compound of formula (IK) with a compound of NHR⁹R¹⁰ to obtain the compound of formula (I):

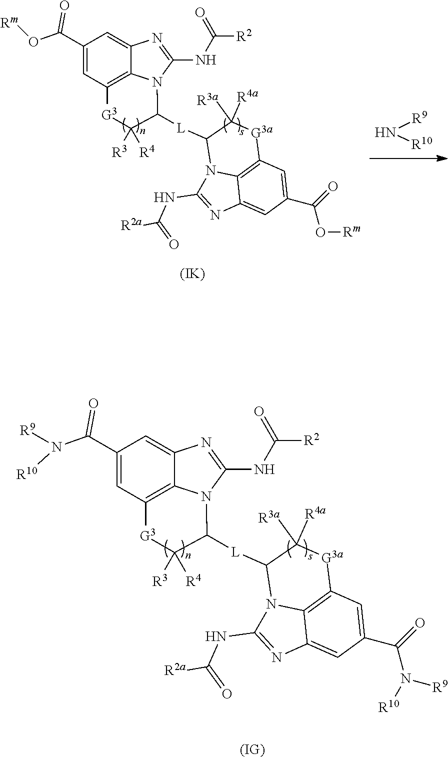

reacting a compound of formula (IK) with a compound of NHR⁹R¹⁰ to obtain the compound of formula (IG);

wherein:

R^{m} is hydrogen or alkyl;

R² to R⁴, R^{2a} to R^{4a}, G³, G^{3a}, R⁹, R¹⁰, n and s are each as defined in formula (IG).

In another aspect, this invention provides a compound of formula (IA) or (IB), used as an intermediate for preparing the compound of formula (I):

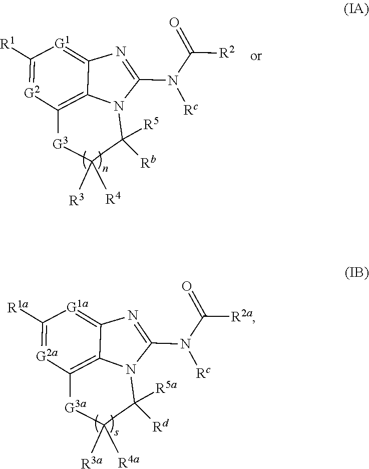

wherein:

R^{b} is —(CH₂)ₚ₁—CH═CR^{e}R^{f};

R^{d} is —(CH₂)ₚ₂—CH═CR^{e}R^{f};

R^{e} and R^{f} are identical or different, and each is independently selected from the group consisting of hydrogen and alkyl;

p¹ is 0, 1, 2 or 3;

p² is 0, 1, 2 or 3;

R¹ to R⁵, R^{1a} to R^{5a}, R^{c}, G¹ to G³, G^{1a} to G^{3a}, n and s are each as defined in formula (I).

Other aspects and advantages of the present invention will be better appreciated in view of the following detailed description, experimental details, and claims.

### DETAILED DESCRIPTION OF THE INVENTION

The present invention relates to a new class of tricyclic compounds useful as STING agonists, preparation methods thereof, and their use as therapeutic agents for treatment of STING-mediated diseases or disorders.

In one aspect, the present invention provides a compound of formula (I), or a pharmaceutically acceptable salt, solvate, or prodrug thereof, including tautomers, cis- or trans-isomers, mesomers, racemates, enantiomers, diastereomers, and mixtures thereof:

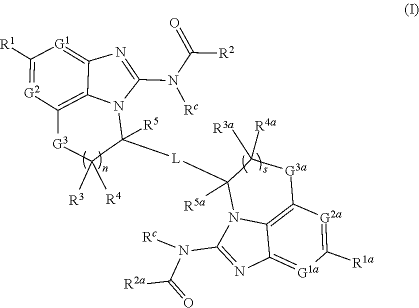

wherein:

G¹, G², G^{1a} and G^{2a} are identical or different, and each is independently N or CR⁶;

G³ and G^{3a} are identical or different, and each is independently O, NR^{g}, or CR⁷R⁸;

L is selected from the group consisting of alkylene, alkenylene, alkynylene, alkylene-Q-alkylene, alkylene-O-alkylene, alkylene-NH-alkylene, alkylene-S(O)ₘ-alkylene, alkylene-C(O)-alkylene, alkylene-C(O)NH-alkylene, alkylene-NHC(O)-alkylene, and alkylene-HNC(O)NH-alkylene, wherein the alkylene, alkenylene, and alkynylene each is unsubstituted or substituted with one or more, sometimes preferably one to five, sometimes more preferably one to three, substituents selected from the group consisting of halogen, alkyl, alkoxy, haloalkyl, amino, nitro, hydroxy, hydroxyalkyl, cyano, cycloalkyl, heterocyclyl, aryl, and heteroaryl;

R^{c} is selected from the group consisting of hydrogen, alkyl, haloalkyl, alkenyl, and alkynyl;

R^{g} is selected from the group consisting of hydrogen, alkyl, cycloalkyl, and alkenyl; wherein the alkyl, cycloalkyl or alkenyl is unsubstituted or substituted with one or more, sometimes preferably one to five, sometimes more preferably one to three, substituents selected from the group consisting of halogen, alkyl, alkoxy, haloalkyl, amino, cyano, hydroxy, hydroxyalkyl, cycloalkyl, heterocyclyl, aryl, and heteroaryl;

R¹ and R^{1a} are identical or different, and each is independently selected from the group consisting of —C(O)NR⁹R¹⁰, —C(O)OR^{m}, hydrogen, halogen, alkyl, alkoxy, haloalkyl, amino, nitro, hydroxy, hydroxyalkyl, cyano, cycloalkyl, heterocyclyl, aryl, and heteroaryl, wherein the alkyl, alkoxy, cycloalkyl, heterocyclyl, aryl or heteroaryl is unsubstituted or substituted with one or more, sometimes preferably one to five, sometimes more preferably one to three, substituents selected from the group consisting of halogen, alkyl, alkoxy, haloalkyl, amino, nitro, hydroxy, hydroxyalkyl, cyano, cycloalkyl, heterocyclyl, aryl, and heteroaryl;

R² and R^{2a} are identical or different, and each is independently selected from the group consisting of alkyl, haloalkyl, cycloalkyl, heterocyclyl, aryl, and heteroaryl; wherein the alkyl, cycloalkyl, heterocyclyl, aryl, or heteroaryl is unsubstituted or substituted with one or more, sometimes preferably one to five, sometimes more preferably one to three, substituents selected from the group consisting of halogen, alkyl, alkoxy, haloalkyl, amino, nitro, cyano, hydroxy, hydroxyalkyl, cycloalkyl, heterocyclyl, aryl, and heteroaryl;

R³, R⁴, R^{3a} and R^{4a} are identical or different, and each is independently selected from the group consisting of hydrogen, halogen, alkyl, alkoxy, haloalkyl, amino, nitro, hydroxy, hydroxyalkyl, cyano, cycloalkyl, heterocyclyl, aryl, and heteroaryl;

R⁵ and R^{5a} are identical or different, and each is independently selected from the group consisting of hydrogen, halogen, alkyl, alkoxy, haloalkyl, amino, nitro, hydroxy, hydroxyalkyl, and cyano;

R⁶ is selected from the group consisting of hydrogen, halogen, alkyl, alkoxy, haloalkyl, amino, nitro, hydroxy, hydroxyalkyl, cyano, cycloalkyl, heterocyclyl, aryl, and heteroaryl;

R⁷ and R⁸ are identical or different, and each is independently selected from the group consisting of hydrogen, halogen, alkyl, alkoxy, haloalkyl, amino, nitro, hydroxy, hydroxyalkyl, cyano, cycloalkyl, heterocyclyl, aryl, and heteroaryl;

R⁹ and R¹⁰ are identical or different, and each is independently selected from the group consisting of hydrogen, alkyl, haloalkyl, hydroxy, cycloalkyl, heterocyclyl, aryl, and heteroaryl; wherein the alkyl, cycloalkyl, heterocyclyl, aryl, or heteroaryl is unsubstituted or substituted with one or more, sometimes preferably one to five, sometimes more preferably one to three, substituents selected from the group consisting of halogen, alkyl, alkoxy, haloalkyl, amino, nitro, cyano, hydroxy, hydroxyalkyl, cycloalkyl, heterocyclyl, aryl, and heteroaryl;

R^{m} is selected from the group consisting of hydrogen, alkyl, haloalkyl, hydroxyalkyl, cycloalkyl, heterocyclyl, aryl, and heteroaryl;

Q is selected from the group consisting of cycloalkyl, heterocyclyl, aryl, and heteroaryl;

m is 0, 1 or 2;

n is 0, 1, 2 or 3; and

s is 0, 1, 2 or 3.

In some embodiments of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, L is selected from the group consisting of alkylene, alkenylene, alkynylene, alkylene-O-alkylene, alkylene-NH-alkylene, alkylene-S(O)ₘ-alkylene, alkylene-C(O)-alkylene, alkylene-C(O)NH— alkylene, alkylene-NHC(O)-alkylene, and alkylene-HNC(O)NH-alkylene, wherein the alkylene or alkenylene each is unsubstituted or substituted with one or more, sometimes preferably one to five, sometimes more preferably one to three, substituents selected from the group consisting of halogen, alkyl, alkoxy, haloalkyl, amino, nitro, hydroxy, hydroxyalkyl, cyano, cycloalkyl, heterocyclyl, aryl, and heteroaryl.

In some embodiments of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, R¹ and R^{1a} are identical or different, and each is independently selected from the group consisting of —C(O)NR⁹R¹⁰, —C(O)OR^{m}, hydrogen, halogen, alkyl, alkoxy, haloalkyl, amino, nitro, hydroxy, hydroxyalkyl, cyano, cycloalkyl, heterocyclyl, aryl, and heteroaryl, wherein the alkyl, alkoxy, cycloalkyl, heterocyclyl, aryl or heteroaryl is unsubstituted or substituted with one or more, sometimes preferably one to five, sometimes more preferably one to three, substituents selected from the group consisting of halogen, alkyl, alkoxy, haloalkyl, amino, nitro, hydroxy, hydroxyalkyl, cyano, cycloalkyl, heterocyclyl, aryl, and heteroaryl; R⁹ and R¹⁰ are as defined in formula (I).

In one embodiment of the invention, the compound of formula (I) is a compound of formula (IM),

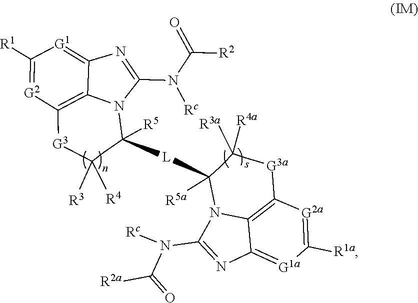

including tautomers, cis- or trans-isomers, mesomers, racemates, enantiomers, diastereomers, or mixtures thereof, or a pharmaceutically acceptable salt, solvate, or prodrug thereof, wherein:

R¹ to R⁵, R^{1a} to R^{5a}, R^{c}, G¹ to G³, G^{1a} to G^{3a}, L, n and s are each as defined in formula (I).

In another embodiment of the invention, the compound of formula (I), when R^{c} is hydrogen, is a compound of formula (I′),

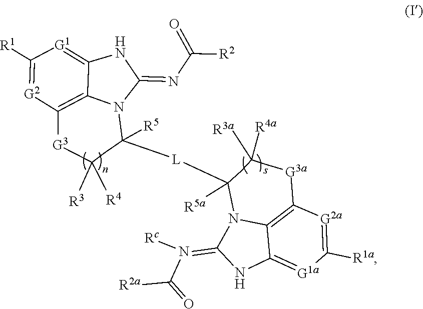

including tautomers, cis- or trans-isomers, mesomers, racemates, enantiomers, diastereomers, or mixtures thereof, or a pharmaceutically acceptable salt, solvate, or prodrug thereof, wherein:

R¹ to R⁵, R^{1a} to R^{5a}, G¹ to G³, G^{1a} to G^{3a}, L, n and s are each as defined in formula (I).

In another embodiment of the invention, the compound of formula (I), when R^{c} is hydrogen, is selected from a compound of formula (I″),

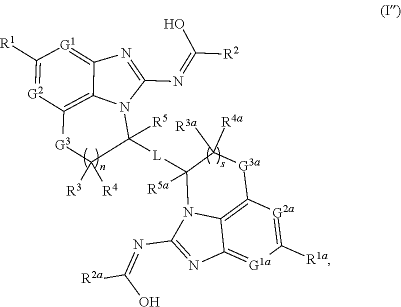

including tautomers, cis- or trans-isomers, mesomers, racemates, enantiomers, diastereomers, or mixtures thereof, or a pharmaceutically acceptable salt, solvate, or prodrug thereof, wherein:

R¹ to R⁵, R^{1a} to R^{5a}, G¹ to G³, G^{1a} to G^{3a}, L, n and s are each as defined in formula (I).

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, G¹, G², G^{1a} and G^{2a} are identical or different, and each is CR⁶, wherein R⁶ is as defined in formula (I).

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, R² and R^{2a} are identical or different, and each is independently selected from the group consisting of aryl and heteroaryl; wherein the aryl or heteroaryl is unsubstituted or substituted with one or more, sometimes preferably one to five, sometimes more preferably one to three, substituents selected from the group consisting of halogen, alkyl, alkoxy, haloalkyl, amino, nitro, cyano, hydroxy, hydroxyalkyl, cycloalkyl, heterocyclyl, aryl, and heteroaryl.

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, R⁵ and R^{5a} are each hydrogen.

In another embodiment of the invention, the compound of formula (I) is a compound of formula (II),

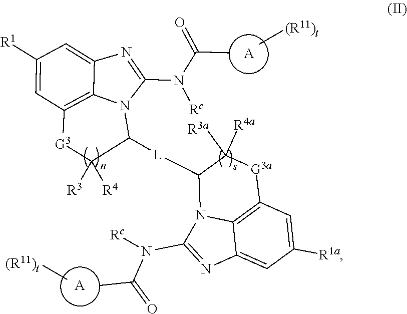

including tautomers, cis- and trans-isomers, mesomers, racemates, enantiomers, diastereomers, or mixtures thereof, or a pharmaceutically acceptable salt, solvate, or prodrug thereof, wherein:

ring A is selected from the group consisting of aryl and heteroaryl;

R¹¹ is each identical or different, and each is independently selected from the group consisting of halogen, alkyl, alkoxy, haloalkyl, amino, nitro, cyano, hydroxy, hydroxyalkyl, cycloalkyl, heterocyclyl, aryl, and heteroaryl;

t is 0, 1, 2, 3 or 4; and

R¹, R^{1a}, R^{c}, R³, R⁴, R^{3a}, R^{4a}, G³, G^{3a}, L, n and s are each as defined in formula (I).

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, R¹ and R^{1a} are identical or different, and each is independently —C(O)NR⁹R¹⁰, wherein R⁹ and R¹⁰ are each as defined in formula (I).

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, R¹ and R^{1a} are identical or different, and each is independently selected from the group consisting of —C(O)NR⁹R¹⁰ and —C(O)OR^{m}, R⁹, R¹⁰ and R^{m} are each as defined in claim 1.

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, R^{c} is hydrogen.

In another embodiment of the invention, the compound of formula (I) is a compound of formula (IG):

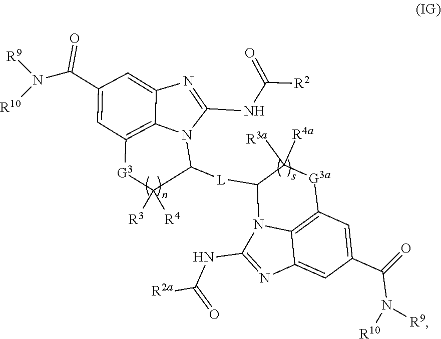

including tautomers, cis- or trans-isomers, mesomers, racemates, enantiomers, diastereomers, or mixtures thereof, or a pharmaceutically acceptable salt, solvate, or prodrug thereof, wherein:

R² to R⁴, R^{2a} to R^{4a}, R⁹, R¹⁰, G³, G^{3a}, L, n and s are each as defined in formula (I).

In another embodiment of the invention, the compound of formula (I) is a compound of formula (IG):

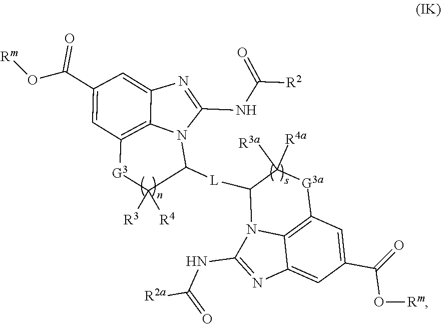

or a tautomer, cis- or trans-isomer, mesomer, racemate, enantiomer, diastereomer, or mixture thereof, or a pharmaceutically acceptable salt, solvate, or prodrug thereof, wherein:

R^{m} is hydrogen or alkyl;

R² to R⁴, R^{2a} to R^{4a}, G³, G^{3a}, L, n and s are each as defined in formula (I).

In another embodiment of the invention, the compound of formula (I) is a compound of formula (III):

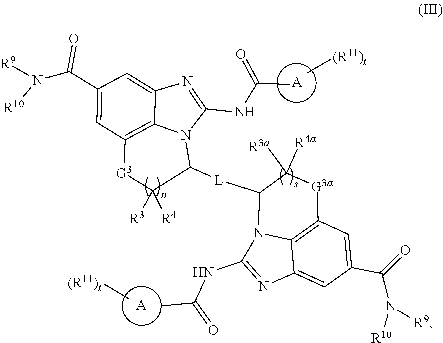

including tautomers, cis- or trans-isomers, mesomers, racemates, enantiomers, diastereomers, or mixtures thereof, or a pharmaceutically acceptable salt, solvate, or prodrug thereof, wherein:

ring A is selected from the group consisting of aryl and heteroaryl;

R¹¹ is each identical or different, and each is independently selected from the group consisting of halogen, alkyl, alkoxy, haloalkyl, amino, nitro, cyano, hydroxy, hydroxyalkyl, cycloalkyl, heterocyclyl, aryl, and heteroaryl;

t is 0, 1, 2, 3 or 4; and

R³, R⁴, R^{3a}, R^{4a}, R⁹, R¹⁰, G³, G^{3a}, L, n and s are each as defined in formula (I).

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, R³, R⁴, R^{3a} and R^{4a} are each hydrogen.

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, G³ and G^{3a} are identical or different, and each is independently O or NH.

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, G³ and G^{3a} are identical or different, and each is independently O or NR^{g}, R^{g} each is identical or different, and each is hydrogen or alkyl, wherein alkyl is unsubstituted or substituted with one or more alkoxy.

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, R⁹ and R¹⁰ are each hydrogen.

In another embodiment of the invention, the compound of formula (I) is a compound of formula (IV):

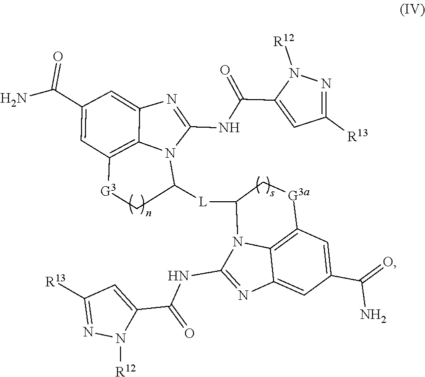

including tautomers, cis- or trans-isomers, mesomers, racemates, enantiomers, diastereomers, or mixtures thereof, or a pharmaceutically acceptable salt, solvate, or prodrug thereof, wherein:

G³ and G^{3a} are identical or different, and each is independently O or NR^{g};

R¹² and R¹³ are identical or different, and each is independently selected from hydrogen and alkyl; and

R^{g}, L, n and s are each as defined in formula (I).

In another embodiment of the invention, the compound of formula (IV) is a compound of formula (IV′):

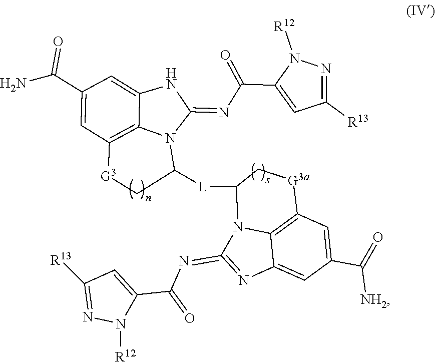

including tautomers, cis- or trans-isomers, mesomers, racemates, enantiomers, diastereomers, or mixtures thereof, or a pharmaceutically acceptable salt, solvate, or prodrug thereof, wherein:

G³, G^{3a}, R¹², R¹³, L, n and s are each as defined in formula (IV).

In another embodiment of the invention, the compound of formula (IV) is a compound of formula (IV″):

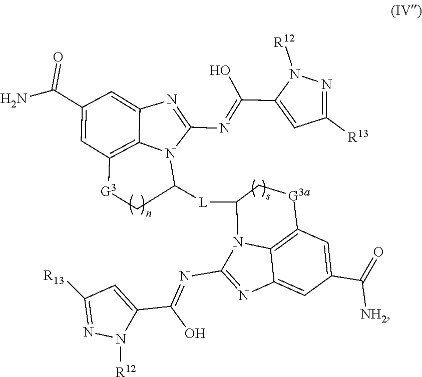

including tautomers, cis- or trans-isomers, mesomers, racemates, enantiomers, diastereomers, or mixtures thereof, or a pharmaceutically acceptable salt, solvate, or prodrug thereof, wherein:

G³, G^{3a}, R¹², R¹³, L, n and s are each as defined in formula (IV).

In another embodiment of the invention, the compound of formula (III) is a compound of formula (IVM),

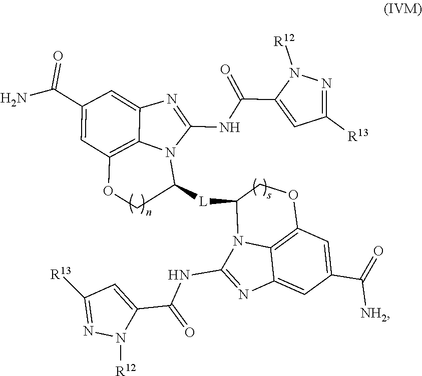

including tautomers, cis- or trans-isomers, mesomers, racemates, enantiomers, diastereomers, or mixtures thereof, or a pharmaceutically acceptable salt, solvate, or prodrug thereof, wherein:

R¹² and R¹³ are identical or different, and each is independently selected from hydrogen and alkyl;

L, n and s are each as defined in formula (I).

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, L is selected from the group consisting of alkylene, alkenylene, and alkylene-O-alkylene, wherein the alkylene or alkenylene each is unsubstituted or substituted with one or more, sometimes preferably one to five, sometimes more preferably one to three, substituents selected from the group consisting of halogen, alkyl, alkoxy, haloalkyl, amino, nitro, hydroxy, hydroxyalkyl, cyano, cycloalkyl, heterocyclyl, aryl, and heteroaryl.

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, L is selected from the group consisting of alkylene, alkenylene, alkynylene, alkylene-Q-alkylene, and alkylene-O-alkylene, wherein the alkylene, alkenylene and alkynylene each is unsubstituted or substituted with one or more substituents selected from the group consisting of halogen, alkyl, alkoxy, haloalkyl, amino, nitro, hydroxy, hydroxyalkyl, cyano, cycloalkyl, heterocyclyl, aryl, and heteroaryl; Q is selected from the group consisting of cycloalkyl, heterocyclyl, aryl, and heteroaryl.

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, L is selected from the group consisting of —(CH₂)ₚ—, —(CH₂)ₚ₁—(CH═CH)_{q}—(CH₂)ₚ₂—, —(CH₂)ₚ₁—O—(CH₂)ₚ₂—, —(CH₂)ₚ₁—(CH(OH))ₜ—(CH₂)ₚ₂—; p is an integer of 1 to 6; p¹ is 0, 1, 2 or 3; p² is 0, 1, 2 or 3; q is 0, 1 or 2; and t is 0, 1, 2 or 3.

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, L is selected from the group consisting of —(CH₂)p-, —(CH₂)p¹-(CH═CH)q-(CH₂)p²-, —(CH₂)p¹-C≡C—(CH₂)p²-, —(CH₂)p¹-cyclopropyl-(CH₂)ₚ²-, —(CH₂)p¹-phenyl-(CH₂)p²-, —(CH₂)p¹-O—(CH₂)p²-, and —(CH₂)p¹-(CH(OH))t-(CH₂)p²-; p is an integer of 1 to 6; p¹ is 0, 1, 2 or 3; p² is 0, 1, 2 or 3; q is 0, 1 or 2; and t is 0, 1, 2 or 3.

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, L is selected from the group consisting of —CH₂—CH═CH—CH₂—, —CH₂CH₂—, —(CH₂)₃—, —(CH₂)₄—, —CH₂CH(OH)CH(OH)CH₂—, —CH₂—CH═CH— and —CH₂—O—CH₂—.

In another embodiment of the invention, in the compound of formula (I), including tautomers, cis- or trans-isomers, mesomers, racemates, enantiomers, diastereomers, or mixtures thereof, or a pharmaceutically acceptable salt, solvate, or prodrug thereof, L is selected from the group consisting of —CH₂—CH═CH—CH₂—, —CH₂CH₂—, —(CH₂)₃—, —(CH₂)₄—, —CH₂CH(OH)CH(OH)CH₂—, —CH₂—CH═CH—, —CH₂-cyclopropyl-CH₂—, —CH₂-phenyl-CH₂—, —CH₂—C≡C—CH₂—, —CH₂—CH═CH—CH₂CH₂—, and —CH₂—O—CH₂—.

Representative compounds of the present invention, or a tautomer, cis- or trans-isomer, mesomer, racemate, enantiomer, diastereomer, or a mixture thereof include, but are not limited to, the compounds listed in Table 1 below.

TABLE 1
Certain exemplified compounds.

| Example | |
|---|---|
| No. | Structure and Name |
| 1 | 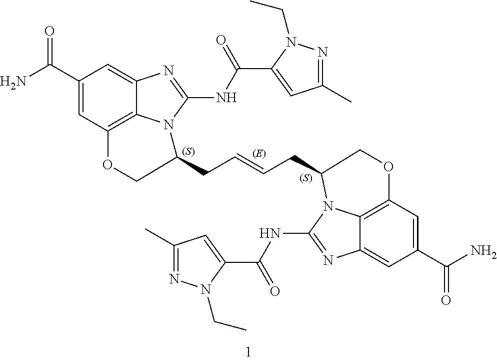 |
| | (3S,3″S)-3,3″-((E)-but-2-ene-1,4-diyl)bis(2-(1-ethyl-3-methyl-1H-pyrazole-5- |
| | carboxamido)-3,4-dihydro-5-oxa-1,2a-diazaacenaphthylene-7-carboxamide) 1 |
| 2 | 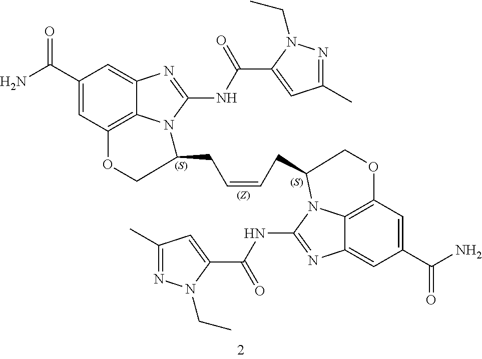 |
| | (3S,3″S)-3,3″-((Z)-but-2-ene-1,4-diyl)bis(2-(1-ethyl-3-methyl-1H-pyrazole-5- |
| | carboxamido)-3,4-dihydro-5-oxa-1,2a-diazaacenaphthylene-7-carboxamide) 2 |
| 2l | 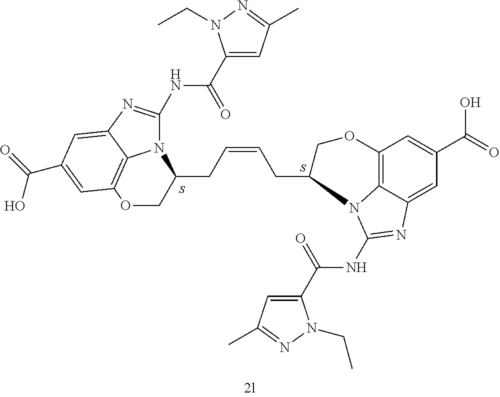 |
| | (3S,3′S)-3,3′-((Z)-but-2-ene-1,4-diyl)bis(2-(1-ethyl-3-methyl-1H-pyrazole-5- |
| | carboxamido)-3,4-dihydro-5-oxa-1,2a-diazaacenaphthylene-7-carboxylic acid) |
| | 2l |
| 2k | 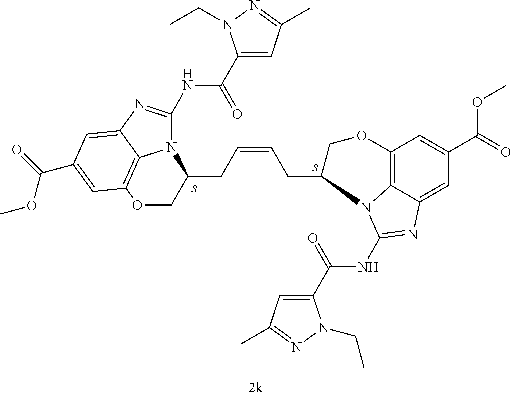 |
| | (3S,3′S)-dimethyl 3,3′-((Z)-but-2-ene-1,4-diyl)bis(2-(1-ethyl-3-methyl-1H- |
| | pyrazole-5-carboxamido)-3,4-dihydro-5-oxa-1,2a-diazaacenaphthylene-7- |
| | carboxylate) 2k |
| 3 | 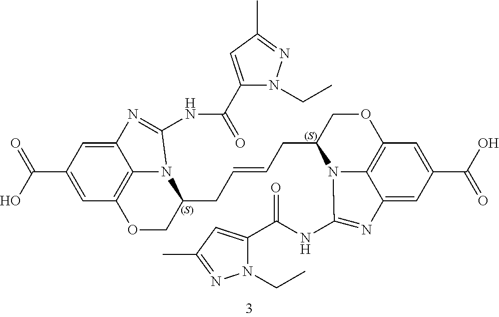 |
| | (3S,3″S)-3,3″-((E)-but-2-ene-1,4-diyl)bis(2-(1-ethyl-3-methyl-1H-pyrazole-5- |
| | carboxamido)-3,4-dihydro-5-oxa-1,2a-diazaacenaphthylene-7-carboxylic acid) |
| | 3 |
| 3l | 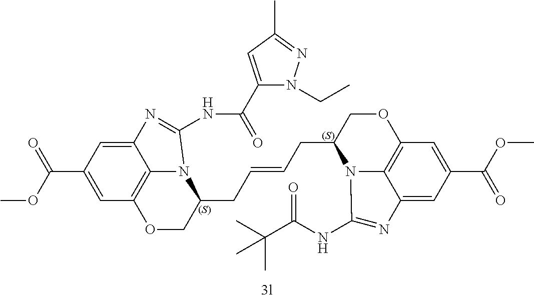 |
| | (S)-methyl 2-(1-ethyl-3-methyl-1H-pyrazole-5-carboxamido)-3-((E)-4-((S)-7- |
| | (methoxycarbonyl)-2-pivalamido-3,4-dihydro-5-oxa-1,2a-diazaacenaphthylen- |
| | 3-yl)but-2-en-1-yl)-3,4-dihydro-5-oxa-1,2a-diazaacenaphthylene-7- |
| | carboxylate 3l |
| 3k | 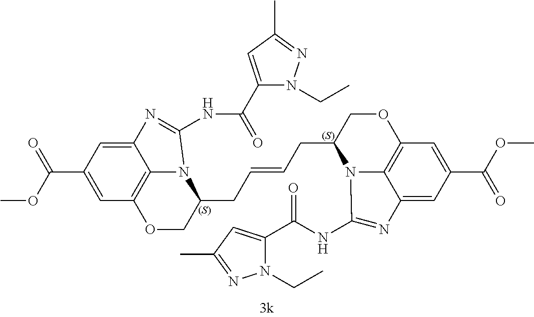 |
| | (3S,3″S)-dimethyl 3,3″-((E)-but-2-ene-1,4-diyl)bis(2-(1-ethyl-3-methyl-1H- |
| | pyrazole-5-carboxamido)-3,4-dihydro-5-oxa-1,2a-diazaacenaphthylene-7- |
| | carboxylate) 3k |
| 3m | 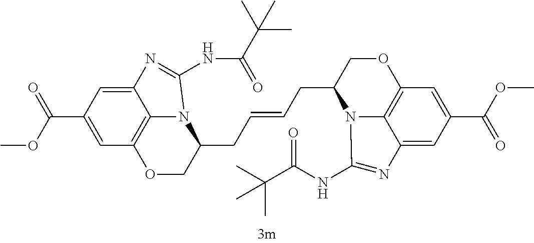 |
| | (3S,3″S)-dimethyl 3,3″-((E)-but-2-ene-1,4-diyl)bis(2-pivalamido-3,4-dihydro- |
| | 5-oxa-1,2a-diazaacenaphthylene-7-carboxylate) 3m |
| 4 | 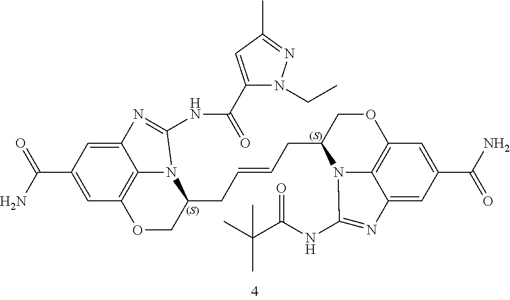 |
| | (S)-3-((E)-4-((S)-7-carbamoyl-2-(1-ethyl-3-methyl-1H-pyrazole-5- |
| | carboxamido)-3,4-dihydro-5-oxa-1,2a-diazaacenaphthylen-3-yl)but-2-en-1- |
| | yl)-2-pivalamido-3,4-dihydro-5-oxa-1,2a-diazaacenaphthylene-7-carboxamide |
| | 4 |
| 4a | 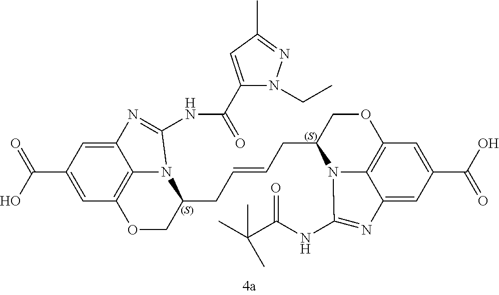 |
| | (S)-3-((E)-4-((S)-7-carboxy-2-(1-ethyl-3-methyl-1H-pyrazole-5- |
| | carboxamido)-3,4-dihydro-5-oxa-1,2a-diazaacenaphthylen-3-yl)but-2-en-1- |
| | yl)-2-pivalamido-3,4-dihydro-5-oxa-1,2a-diazaacenaphthylene-7-carboxylic |
| | acid 4a |
| 5 | 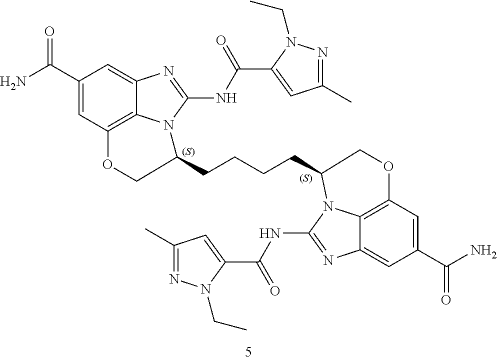 |
| | (3S,3″S)-3,3″-(butane-1,4-diyl)bis(2-(1-ethyl-3-methyl-1H-pyrazole-5- |
| | carboxamido)-3,4-dihydro-5-oxa-1,2a-diazaacenaphthylene-7-carboxamide) 5 |
| 6 | 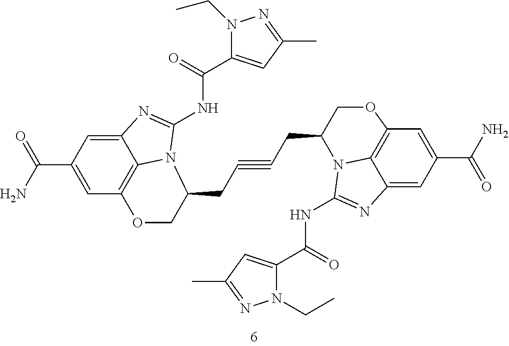 [SMILES: CCn1nc(C)cc1C(=O)Nc1nc2cc(C(N)=O)cc3c2n1[C@@H](CC#CC[C@H]1COc2cc(C(N)=O)cc4nc(NC(=O)c5cc(C)nn5CC)n1c24)CO3] |
| | (3S,3″S)-3,3″-(but-2-yne-1,4-diyl)bis(2-(1-ethyl-3-methyl-1H-pyrazole-5- |
| | carboxamido)-3,4-dihydro-5-oxa-1,2a-diazaacenaphthylene-7-carboxamide) 6 |
| 6j | 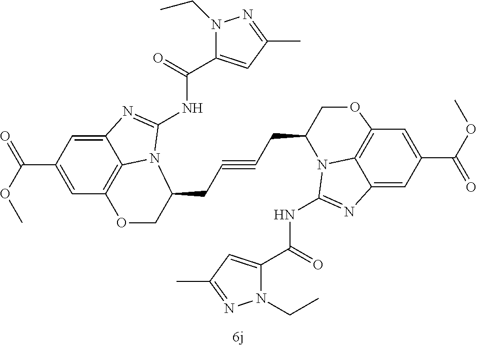 [SMILES: CCn1nc(C)cc1C(=O)Nc1nc2cc(C(=O)OC)cc3c2n1[C@@H](CC#CC[C@H]1COc2cc(C(=O)OC)cc4nc(NC(=O)c5cc(C)nn5CC)n1c24)CO3] |
| | (3S,3′S)-dimethyl 3,3′-(but-2-yne-1,4-diyl)bis(2-(1-ethyl-3-methyl-1H- |
| | pyrazole-5-carboxamido)-3,4-dihydro-5-oxa-1,2a-diazaacenaphthylene-7- |
| | carboxylate) 6j |
| 6k | 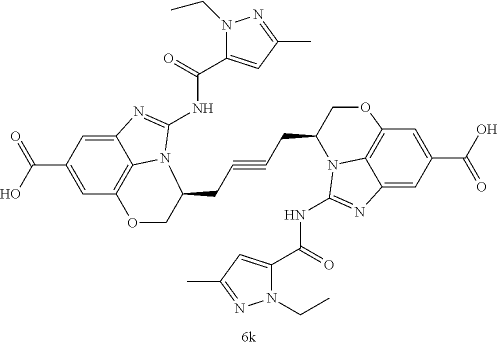 [SMILES: CCn1nc(C)cc1C(=O)Nc1nc2cc(C(=O)O)cc3c2n1[C@@H](CC#CC[C@H]1COc2cc(C(=O)O)cc4nc(NC(=O)c5cc(C)nn5CC)n1c24)CO3] |
| | (3S,3″S)-3,3″-(but-2-yne-1,4-diyl)bis(2-(1-ethyl-3-methyl-1H-pyrazole-5- |
| | carboxamido)-3,4-dihydro-5-oxa-1,2a-diazaacenaphthylene-7-carboxylic acid) |
| | 6k |
| 7 | 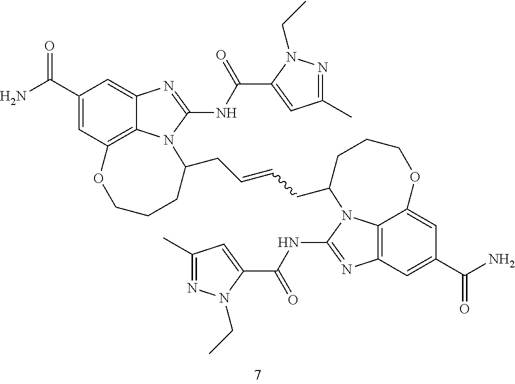 [SMILES: CCn1nc(C)cc1C(=O)Nc1nc2cc(C(N)=O)cc3c2n1C(CC=CCC1CCCOc2cc(C(N)=O)cc4nc(NC(=O)c5cc(C)nn5CC)n1c24)CCCO3] |
| | 10,10″-(but-2-ene-1,4-diyl)bis(1-(1-ethyl-3-methyl-1H-pyrazole-5- |
| | carboxamido)-7,8,9,10-tetrahydro-6-oxa-2,10a-diazacycloocta[cd]indene-4- |
| | carboxamide) 7 |
| 8 | 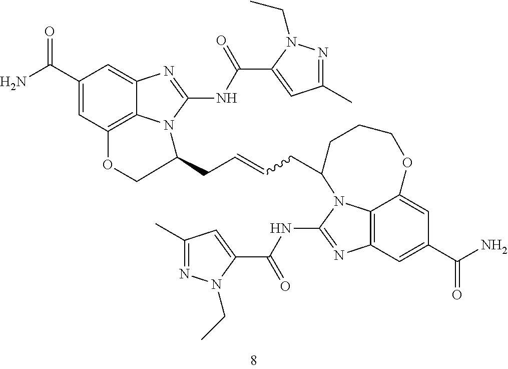 [SMILES: CCn1nc(C)cc1C(=O)Nc1nc2cc(C(N)=O)cc3c2n1C(CC=CC[C@H]1COc2cc(C(N)=O)cc4nc(NC(=O)c5cc(C)nn5CC)n1c24)CCCO3] |
| | 10-(4-((S)-7-carbamoyl-2-(1-ethyl-3-methyl-1H-pyrazole-5-carboxamido)- |
| | 3,4-dihydro-5-oxa-1,2a-diazaacenaphthylen-3-yl)but-2-en-1-yl)-1-(1-ethyl-3- |
| | methyl-1H-pyrazole-5-carboxamido)-7,8,9,10-tetrahydro-6-oxa-2,10a- |
| | diazacycloocta[cd]indene-4-carboxamide 8 |
| 9 | 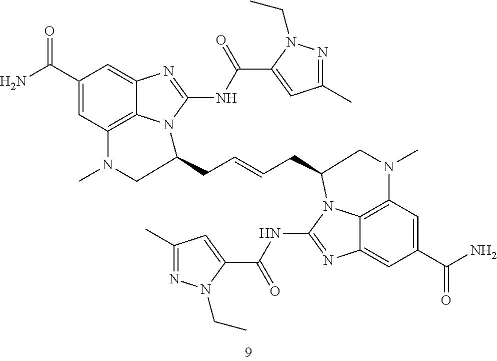 [SMILES: CCn1nc(C)cc1C(=O)Nc1nc2cc(C(N)=O)cc3c2n1[C@@H](C/C=C/C[C@H]1CN(C)c2cc(C(N)=O)cc4nc(NC(=O)c5cc(C)nn5CC)n1c24)CN3C] |
| | (4S,4′S)-4,4′-((E)-but-2-ene-1,4-diyl)bis(2-(1-ethyl-3-methyl-1H-pyrazole-5- |
| | carboxamido)-6-methyl-5,6-dihydro-4H-imidazo[1,5,4-de]quinoxaline-8- |
| | carboxamide) 9 |
| 10 | 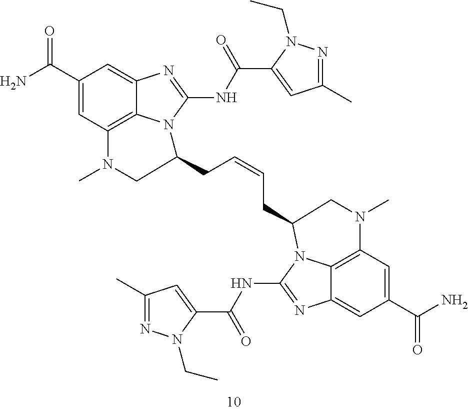 [SMILES: CCn1nc(C)cc1C(=O)Nc1nc2cc(C(N)=O)cc3c2n1[C@@H](C/C=C\C[C@H]1CN(C)c2cc(C(N)=O)cc4nc(NC(=O)c5cc(C)nn5CC)n1c24)CN3C] |
| | (4S,4′S)-4,4′-((Z)-but-2-ene-1,4-diyl)bis(2-(1-ethyl-3-methyl-1H-pyrazole-5- |
| | carboxamido)-6-methyl-5,6-dihydro-4H-imidazo[1,5,4-de]quinoxaline-8- |
| | carboxamide) 10 |
| 11 | 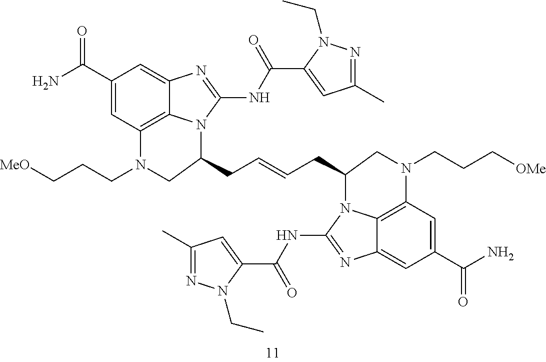 [SMILES: CCn1nc(C)cc1C(=O)Nc1nc2cc(C(N)=O)cc3c2n1[C@@H](C/C=C/C[C@H]1CN(CCCOC)c2cc(C(N)=O)cc4nc(NC(=O)c5cc(C)nn5CC)n1c24)CN3CCCOC] |
| | (4S,4′S)-4,4′-((E)-but-2-ene-1,4-diyl)bis(2-(1-ethyl-3-methyl-1H-pyrazole-5- |
| | carboxamido)-6-(3-methoxypropyl)-5,6-dihydro-4H-imidazo[1,5,4- |
| | de]quinoxaline-8-carboxamide) 11 |
| 12 | 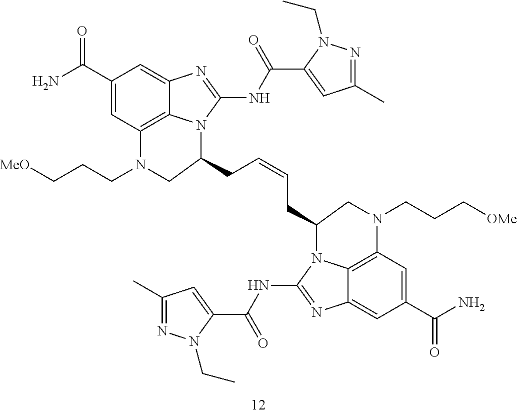 [SMILES: CCn1nc(C)cc1C(=O)Nc1nc2cc(C(N)=O)cc3c2n1[C@@H](C/C=C\C[C@H]1CN(CCCOC)c2cc(C(N)=O)cc4nc(NC(=O)c5cc(C)nn5CC)n1c24)CN3CCCOC] |
| | (4S,4′S)-4,4′-((Z)-but-2-ene-1,4-diyl)bis(2-(1-ethyl-3-methyl-1H-pyrazole-5- |
| | carboxamido)-6-(3-methoxypropyl)-5,6-dihydro-4H-imidazo[1,5,4- |
| | de]quinoxaline-8-carboxamide) 12 |
| 13 | 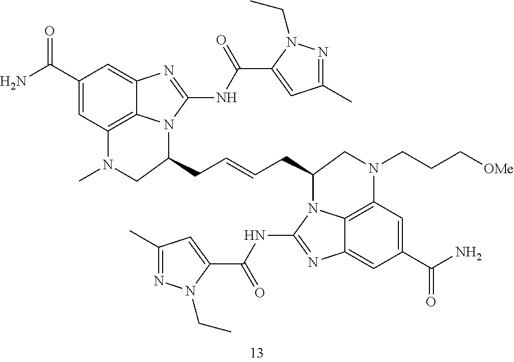 [SMILES: CCn1nc(C)cc1C(=O)Nc1nc2cc(C(N)=O)cc3c2n1[C@@H](C/C=C/C[C@H]1CN(CCCOC)c2cc(C(N)=O)cc4nc(NC(=O)c5cc(C)nn5CC)n1c24)CN3C] |
| | (S)-4-((E)-4-((S)-8-carbamoyl-2-(1-ethyl-3-methyl-1H-pyrazole-5- |
| | carboxamido)-6-(3-methoxypropyl)-5,6-dihydro-4H-imidazo[1,5,4- |
| | de]quinoxalin-4-yl)but-2-en-1-yl)-2-(1-ethyl-3-methyl-1H-pyrazole-5- |
| | carboxamido)-6-methyl-5,6-dihydro-4H-imidazo[1,5,4-de]quinoxaline-8- |
| | carboxamide 13 |
| 14 | 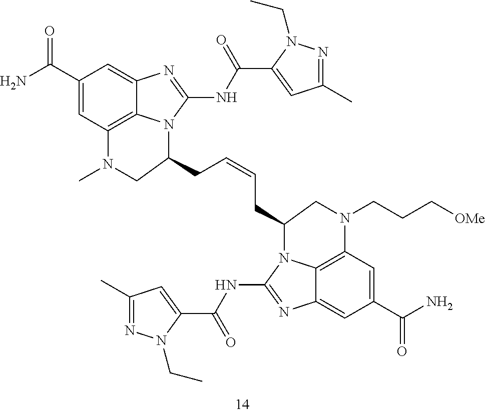 [SMILES: CCn1nc(C)cc1C(=O)Nc1nc2cc(C(N)=O)cc3c2n1[C@@H](C/C=C\C[C@H]1CN(CCCOC)c2cc(C(N)=O)cc4nc(NC(=O)c5cc(C)nn5CC)n1c24)CN3C] |
| | (S)-4-((Z)-4-((S)-8-carbamoyl-2-(1-ethyl-3-methyl-1H-pyrazole-5- |
| | carboxamido)-6-(3-methoxypropyl)-5,6-dihydro-4H-imidazo[1,5,4- |
| | de]quinoxalin-4-yl)but-2-en-1-yl)-2-(1-ethyl-3-methyl-1H-pyrazole-5- |
| | carboxamido)-6-methyl-5,6-dihydro-4H-imidazo[1,5,4-de]quinoxaline-8- |
| | carboxamide 14 |
| 15 | 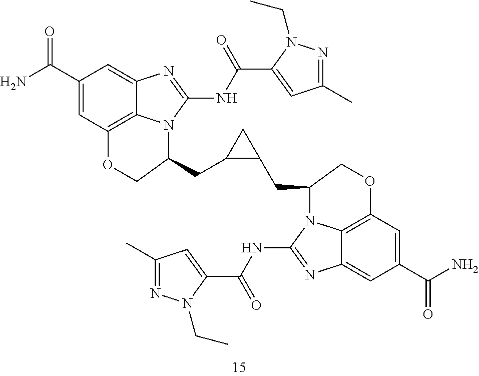 [SMILES: CCn1nc(C)cc1C(=O)Nc1nc2cc(C(N)=O)cc3c2n1[C@@H](CC1CC1C[C@H]1COc2cc(C(N)=O)cc4nc(NC(=O)c5cc(C)nn5CC)n1c24)CO3] |
| | (3S,3″S)-3,3″-(cyclopropane-1,2-diylbis(methylene))bis(2-(1-ethyl-3-methyl- |
| | 1H-pyrazole-5-carboxamido)-3,4-dihydro-5-oxa-1,2a-diazaacenaphthylene-7- |
| | carboxamide) 15 |
| 16 | 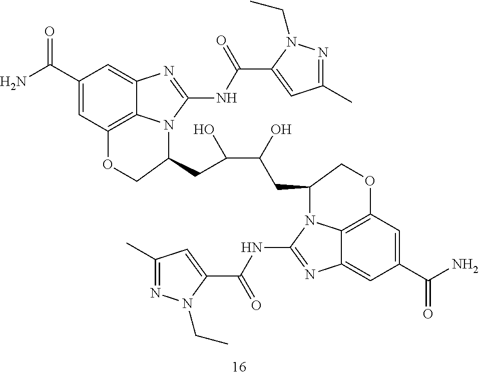 [SMILES: CCn1nc(C)cc1C(=O)Nc1nc2cc(C(N)=O)cc3c2n1[C@@H](CC(O)C(O)C[C@H]1COc2cc(C(N)=O)cc4nc(NC(=O)c5cc(C)nn5CC)n1c24)CO3] |
| | (3S,3″S)-3,3″-(2,3-dihydroxybutane-1,4-diyl)bis(2-(1-ethyl-3-methyl-1H- |
| | pyrazole-5-carboxamido)-3,4-dihydro-5-oxa-1,2a-diazaacenaphthylene-7- |
| | carboxamide) 16 |
| 17 | 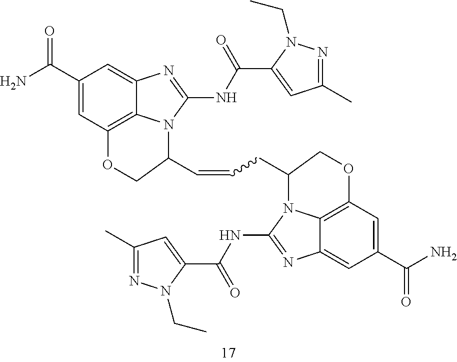 [SMILES: CCn1nc(C)cc1C(=O)Nc1nc2cc(C(N)=O)cc3c2n1C(C=CCC1COc2cc(C(N)=O)cc4nc(NC(=O)c5cc(C)nn5CC)n1c24)CO3] |
| | 3,3″-(prop-1-ene-1,3-diyl)bis(2-(1-ethyl-3-methyl-1H-pyrazole-5- |
| | carboxamido)-3,4-dihydro-5-oxa-1,2a-diazaacenaphthylene-7-carboxamide) |
| | 17 |
| 18 | 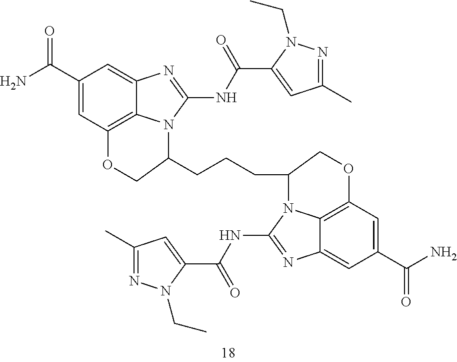 [SMILES: CCn1nc(C)cc1C(=O)Nc1nc2cc(C(N)=O)cc3c2n1C(CCCC1COc2cc(C(N)=O)cc4nc(NC(=O)c5cc(C)nn5CC)n1c24)CO3] |
| | 3,3″-(propane-1,3-diyl)bis(2-(1-ethyl-3-methyl-1H-pyrazole-5-carboxamido)- |
| | 3,4-dihydro-5-oxa-1,2a-diazaacenaphthylene-7-carboxamide) 18 |
| 19 | 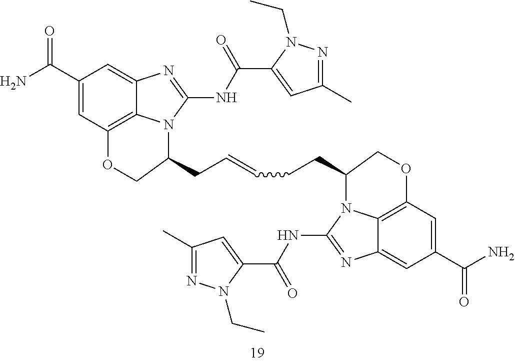 [SMILES: CCn1nc(C)cc1C(=O)Nc1nc2cc(C(N)=O)cc3c2n1[C@@H](CC=CCC[C@H]1COc2cc(C(N)=O)cc4nc(NC(=O)c5cc(C)nn5CC)n1c24)CO3] |
| | (3S,3″S)-3,3″-(pent-2-ene-1,5-diyl)bis(2-(1-ethyl-3-methyl-1H-pyrazole-5- |
| | carboxamido)-3,4-dihydro-5-oxa-1,2a-diazaacenaphthylene-7-carboxamide) |
| | 19 |
| 20 | 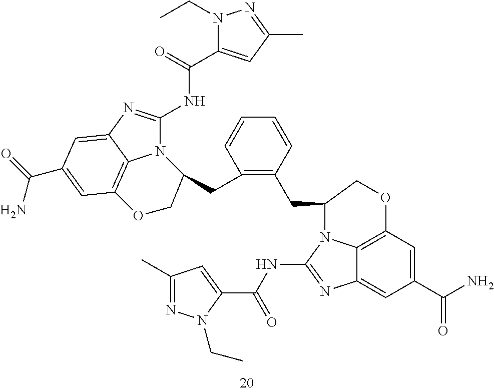 [SMILES: CCn1nc(C)cc1C(=O)Nc1nc2cc(C(N)=O)cc3c2n1[C@@H](Cc1ccccc1C[C@H]1COc2cc(C(N)=O)cc4nc(NC(=O)c5cc(C)nn5CC)n1c24)CO3] |
| | (3S,3″S)-3,3″-(1,2-phenylenebis(methylene))bis(2-(1-ethyl-3-methyl-1H- |
| | pyrazole-5-carboxamido)-3,4-dihydro-5-oxa-1,2a-diazaacenaphthylene-7- |
| | carboxamide) 20 |

In another aspect, this invention provides a compound of formula (IA) or (IB), or a tautomer, cis- or trans-isomer, mesomer, racemate, enantiomer, diastereomer, or mixture thereof, used as an intermediate for preparing a compound of formula (I), wherein:

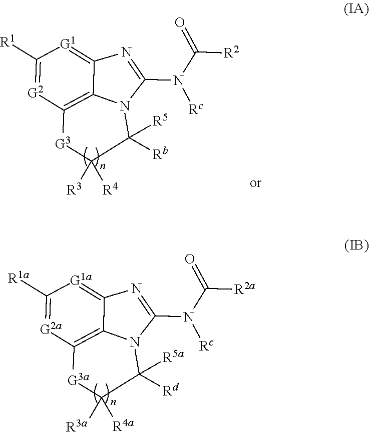

wherein:

R^{b} is —(CH₂)ₚ₁—CH═CR^{e}R^{f};

R^{d} is —(CH₂)ₚ₂—CH═CR^{e}R^{f};

R^{e} and R^{f} are identical or different, and each is independently selected from the group consisting of hydrogen and alkyl;

p¹ is 0, 1, 2 or 3;

p² is 0, 1, 2 or 3;

R¹ to R⁵, R^{1a} to R^{5a}, R^{c}, G¹ to G³, G^{1a} to G^{3a}, n and s are each as defined in formula (I).

Representative intermediates of the present invention include, but are not limited to, the compounds listed in Table 2 below.

TABLE 2
Certain exemplified intermediate compounds.

| Example | |
|---|---|
| No. | Structure and Name |
| 1k | 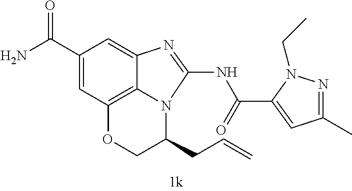 [SMILES: C=CC[C@H]1COc2cc(C(N)=O)cc3nc(NC(=O)c4cc(C)nn4CC)n1c23] |
| | (S)-3-allyl-2-(1-ethyl-3-methyl-1H-pyrazole-5-carboxamido)- |
| | 3,4-dihydro-5-oxa-1,2a-diazaacenaphthylene-7- |
| | carboxamide 1k |
| 7p | 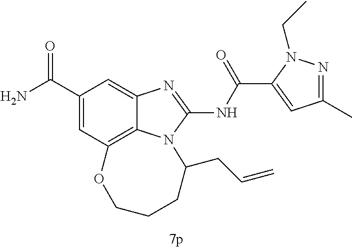 [SMILES: C=CCC1CCCOc2cc(C(N)=O)cc3nc(NC(=O)c4cc(C)nn4CC)n1c23] |
| | 10-allyl-1-(1-ethyl-3-methyl-1H-pyrazole-5- |
| | carboxamido)-7,8,9,10-tetrahydro-6-oxa-2,10a- |
| | diazacycloocta[cd]indene-4-carboxamide 7p |
| 9n | 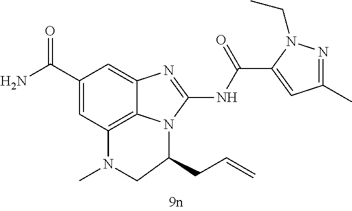 [SMILES: C=CC[C@H]1CN(C)c2cc(C(N)=O)cc3nc(NC(=O)c4cc(C)nn4CC)n1c23] |
| | (S)-4-allyl-2-(1-ethyl-3-methyl-1H-pyrazole-5- |
| | carboxamido)-6-methyl-5,6-dihydro-4H-imidazo |
| | [1,5,4-de]quinoxaline-8-carboxamide 9n |
| 11f | 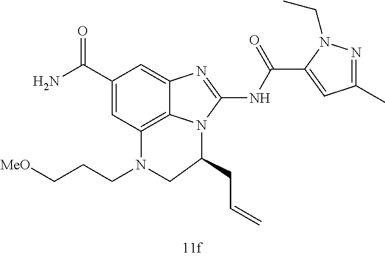 [SMILES: C=CC[C@H]1CN(CCCOC)c2cc(C(N)=O)cc3nc(NC(=O)c4cc(C)nn4CC)n1c23] |
| | (S)-4-allyl-2-(1-ethyl-3-methyl-1H-pyrazole-5-carboxamido)-6- |
| | (3-methoxypropyl)-5,6-dihydro-4H-imidazo[1,5,4-de] |
| | quinoxaline-8-carboxamide 11f |

or a tautomer, cis- or trans-isomer, racemate, enantiomer, or mixture thereof, or a pharmaceutically acceptable salt thereof.

In another aspect, this invention provides a preparation process of a compound of formula (I), or a tautomer, cis- or trans-isomer, mesomer, racemate, enantiomer, diastereomer, or mixture thereof, or a pharmaceutically acceptable salt thereof, the preparation process comprising a step of:

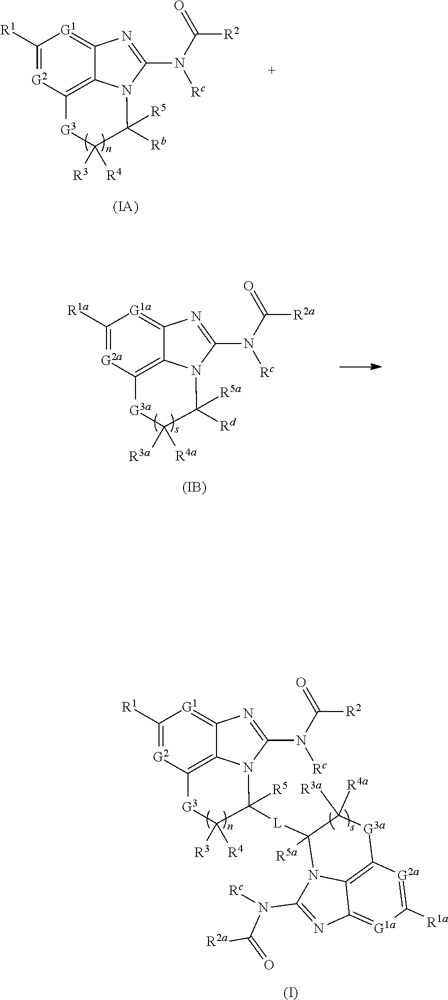

reacting a compound of formula (IA) with a compound of formula (IB) to obtain the compound of formula (I), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3; and

R¹ to R⁵, R^{1a} to R^{5a}, R^{c}, G¹ to G³, G^{1a} to G^{3a}, n and s are each as defined in formula (I).

In another aspect, this invention provides a preparation process of a compound of formula (IM), or a tautomer, cis- or trans-isomer, mesomer, racemate, enantiomer, diastereomer, or mixture thereof, or a pharmaceutically acceptable salt thereof, the preparation process comprising a step of:

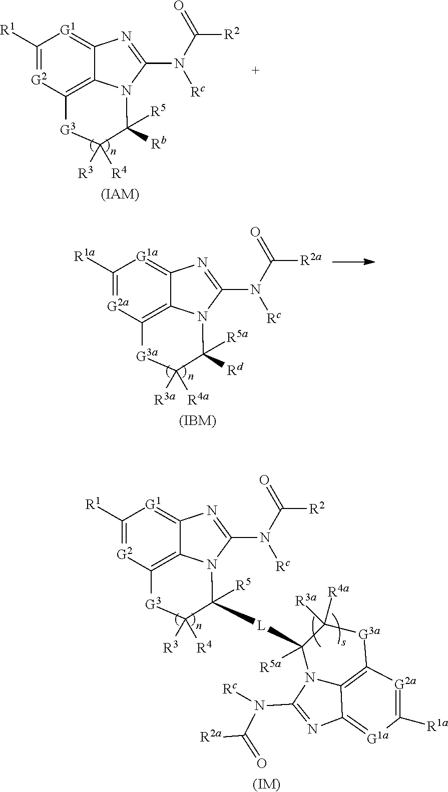

reacting a compound of formula (IAM) with a compound of formula (IBM) to obtain the compound of formula (IM), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3; and

R¹ to R⁵, R^{1a} to R^{5a}, R^{c}, G¹ to G³, G^{1a} to G^{3a}, n and s are each as defined in formula (IM).

In another aspect, this invention provides a preparation process of a compound of formula (II), or a tautomer, cis- or trans-isomer, mesomer, racemate, enantiomer, diastereomer, or mixture thereof, or a pharmaceutically acceptable salt thereof, the preparation process comprising a step of:

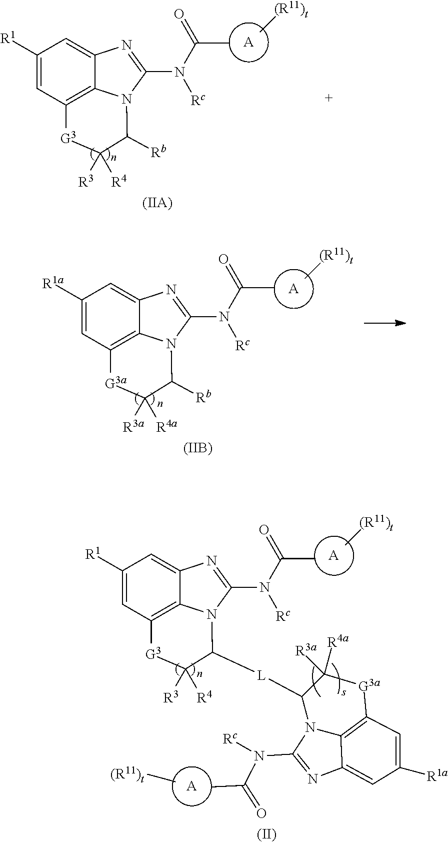

reacting a compound of formula (IIA) with a compound of formula (IIB) to obtain the compound of formula (II), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3;

ring A, R¹, R^{1a}, R^{c}, R³, R⁴, R^{3a}, R^{4a}, R¹¹, G³, G^{3a}, t, n and s are each as defined in formula (II).

In another aspect, this invention provides a preparation process of a compound of formula (III), or a tautomer, cis- or trans-isomer, mesomer, racemate, enantiomer, diastereomer, or mixture thereof, or a pharmaceutically acceptable salt thereof, the preparation process comprising a step of:

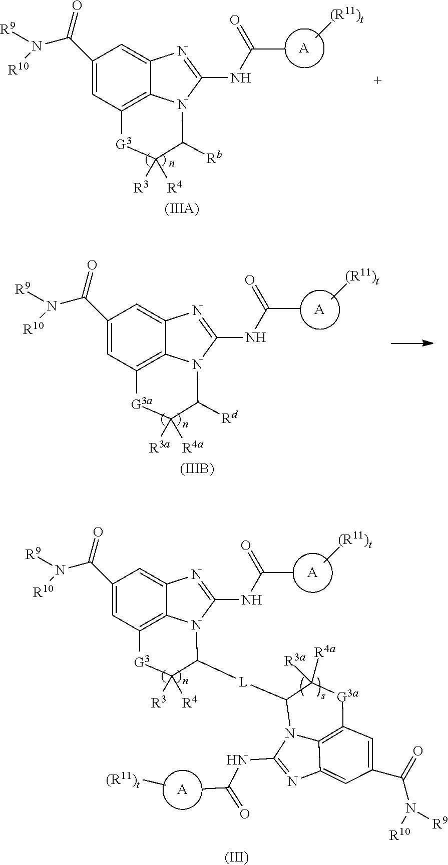

reacting a compound of formula (IIIA) with a compound of formula (IIIB) to obtain the compound of formula (III), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3; and

ring A, R³, R⁴, R^{3a}, R^{4a}, R⁹ to R¹¹, G³, G^{3a}, t, n and s are each as defined in formula (III).

In another aspect, this invention provides a preparation process of a compound of formula (IG), or a tautomer, cis- or trans-isomer, mesomer, racemate, enantiomer, diastereomer, or mixture thereof, or a pharmaceutically acceptable salt thereof, the preparation process comprising a step of:

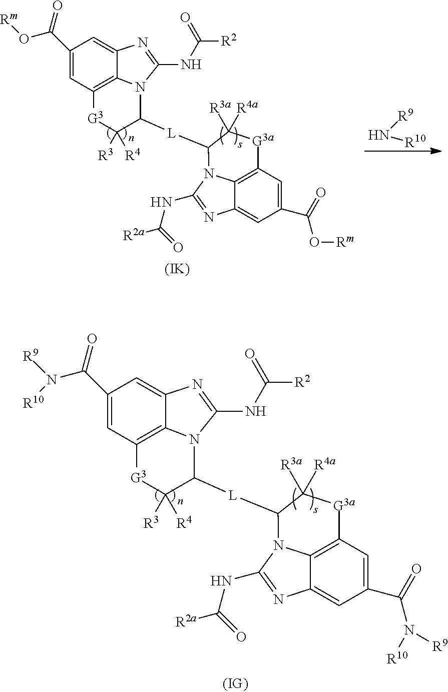

reacting a compound of formula (IK) with a compound of NHR⁹R¹⁰ to obtain the compound of formula (IG), wherein:

R^{m} is hydrogen or alkyl;

R² to R⁴, R^{2a} to R^{4a}, G³, G^{3a}, R⁹, R¹⁰, n and s are each as defined in formula (IG).

In another aspect, this invention provides a preparation process of a compound of formula (III), or a tautomer, cis- or trans-isomer, mesomer, racemate, enantiomer, diastereomer, or mixture thereof, or a pharmaceutically acceptable salt thereof, the preparation process comprising a step of:

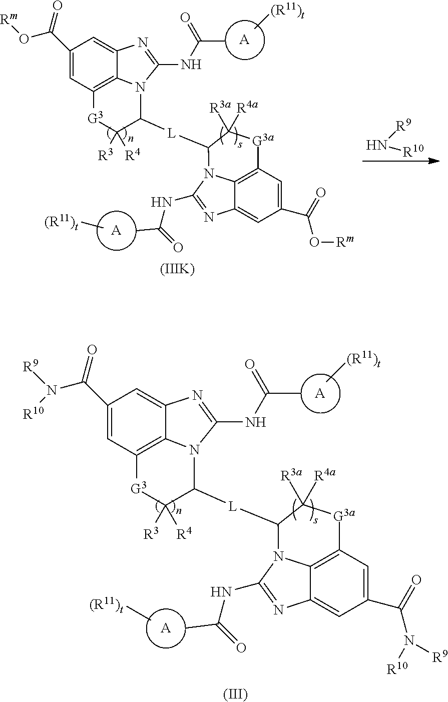

reacting a compound of formula (IIIK) with a compound of NHR⁹R¹⁰ to obtain the compound of formula (III), wherein:

R^{m} is hydrogen or alkyl;

Ring A, R³, R⁴, R^{3a}, R^{4a}, G³, G^{3a}, R⁹˜R¹¹, t, n and s are each as defined in formula (III).

In another aspect, this invention provides a preparation process of a compound of formula (IV), or a tautomer, cis- or trans-isomer, mesomer, racemate, enantiomer, diastereomer, or mixture thereof, or a pharmaceutically acceptable salt thereof, the preparation process comprising a step of:

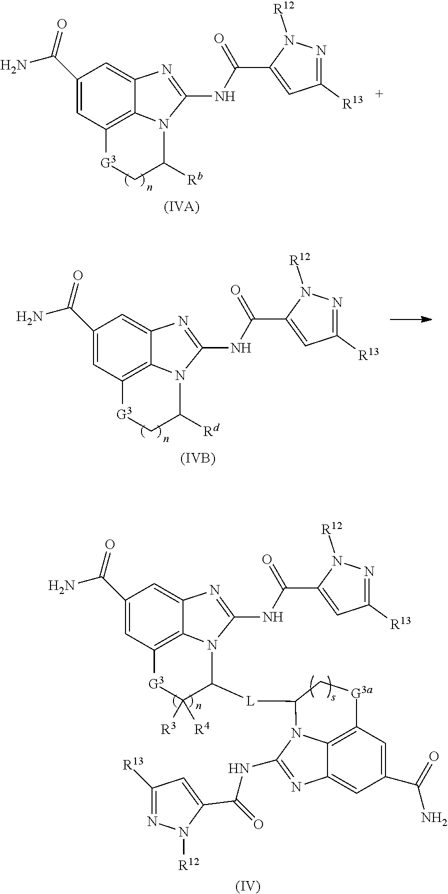

reacting a compound of formula (IVA) with a compound of formula (IVB) to obtain the compound of formula (IV), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3; and

R¹², R¹³, G³, G^{3a}, n and s are each as defined in formula (IV).

In another aspect, this invention provides a preparation process of a compound of formula (IVM), or a tautomer, cis- or trans-isomer, mesomer, racemate, enantiomer, diastereomer, or mixture thereof, or a pharmaceutically acceptable salt thereof, the preparation process comprising a step of:

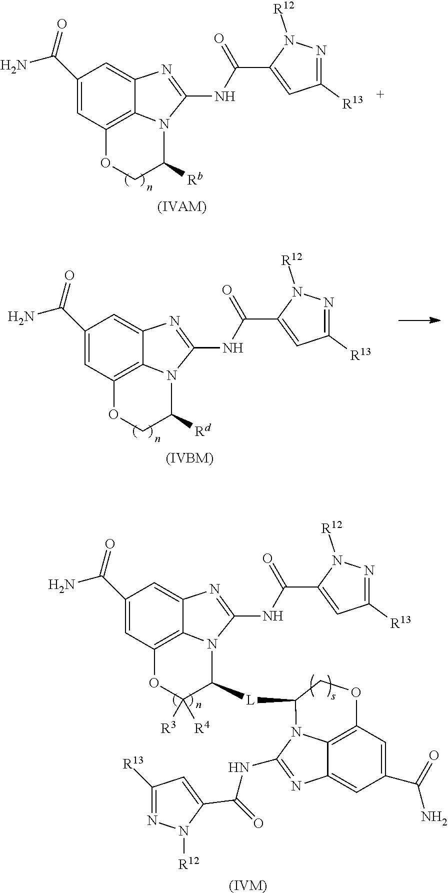

reacting a compound of formula (IVAM) with a compound of formula (IVBM) to obtain the compound of formula (IVM), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3; and

R¹², R¹³, G^{3a}, n and s are each as defined in formula (IVM).

The present invention also provides a pharmaceutical composition, comprising a therapeutically effective amount of a compound of formula (I), in any embodiment disclosed herein, or a tautomer, cis- or trans isomer, mesomer, racemate, enantiomer, diastereomer, or mixture thereof, or a pharmaceutically acceptable salt, solvate, or prodrug thereof, together with one or more pharmaceutically acceptable carriers, diluents, and/or other excipients.

The present invention also relates to use of a compound of formula (I), in any embodiment disclosed herein, or a tautomer, cis- or trans-isomer, mesomer, racemate, enantiomer, diastereomer, or mixture thereof, or a pharmaceutically acceptable salt, solvate, or prodrug thereof, in the preparation of a medicament for use as STING agonist.

The present invention also relates to use of a compound of formula (I), in any embodiment disclosed herein, or a tautomer, cis- or trans-isomer, mesomer, racemate, enantiomer, diastereomer, or mixture thereof, or a pharmaceutically acceptable salt, solvate, or prodrug thereof, or a pharmaceutical composition comprising the same, in the preparation of a medicament for the treatment of a STING-mediated disease or disorder.

In other words, the present invention relates to a method for stimulating STING, comprising a step of administering to a subject in need thereof a therapeutically effective amount of a compound of formula (I), in any embodiment disclosed herein, or a tautomer, racemate, enantiomer, diastereoisomer, or mixture thereof, or a pharmaceutically acceptable salt, solvate, or prodrug thereof, or a pharmaceutical composition containing the same.

The present invention relates to a method for treating a STING-mediated disease or disorder, comprising a step of administering to a subject in need thereof a therapeutically effective amount of a compound of formula (I), or a tautomer, cis- or trans-isomer, mesomer, racemate, enantiomer, diastereomer, or mixture thereof, or a pharmaceutically acceptable salt, solvate, or prodrug thereof, or a pharmaceutical composition comprising the same.

In one embodiment, the disease or disorder is selected from a cancer, a pre-cancerous syndrome and viral infections, preferably a cancer and a pre-cancerous syndrome.

In one embodiment, the disease or disorder is brain cancer, leukemia, skin cancer (e.g., melanoma), prostate cancer, thyroid cancer, colon cancer, lung cancer, breast cancer, or sarcoma. In another embodiment the cancer is selected from the group consisting of glioma, glioblastoma multiforme, paraganglioma, suprantentorial primordial neuroectodermal tumors, acute myeloid leukemia (AML), myelodysplastic syndrome (MDS), chronic myelogenous leukemia (CML), myeloproliferative neoplasm (MPN), angioimmunoblastic lymphoma, melanoma, breast, prostate, thyroid, colon, lung, central chondrosarcoma, central and periosteal chondroma tumors, fibrosarcoma, and cholangiocarcinoma.

Disclosed herein is also use of a compound of general formula (I), in any embodiment disclosed herein, or a pharmaceutically acceptable salt, solvate, or prodrug thereof, for treating a viral infection or cell proliferation disorder, such as cancer, in combination with administration of one or more additional active agents, for example, STING agonist compounds, anti-viral agents, anti-cancer agents, antigens, adjuvants, CTLA-4, LAG-3 and PD-1 pathway antagonists, cytotoxic agents, chemotherapeutic agents, checkpoint inhibitors, vascular endothelial growth factor (VEGF) receptor inhibitors, alkylating agents, anti-tumor antibiotics, retinoids, and immunomodulatory agents.

The compositions of this invention can be formulated by conventional methods using one or more pharmaceutically acceptable carriers. Thus, the active compounds of this invention can be formulated as various dosage forms for oral, buccal, intranasal, parenteral (e.g., intravenous, intramuscular or subcutaneous), rectal administration, inhalation or insufflation administration. The compounds of this invention can also be formulated as sustained release dosage forms.

Oral compositions include a tablet, troche, lozenge, aqueous or oily suspension, dispersible powder or granule, emulsion, hard or soft capsule, or syrup or elixir. Oral compositions can be prepared according to any known method in the art for the preparation of pharmaceutical compositions. Such compositions can contain one or more additives selected from the group consisting of sweeteners, flavoring agents, colorants and preservatives, in order to provide a pleasing and palatable pharmaceutical preparation. Tablets contain the active ingredient and nontoxic pharmaceutically acceptable excipients suitable for the manufacture of tablets. These excipients can be inert excipients, granulating agents, disintegrating agents, and lubricants. The tablet can be uncoated or coated by means of a known technique to mask the taste of the drug or delay the disintegration and absorption of the drug in the gastrointestinal tract, thereby providing sustained release over an extended period. For example, water soluble taste masking materials can be used.

Oral formulations can also be provided as soft gelatin capsules in which the active ingredient is mixed with an inert solid diluent, or the active ingredient is mixed with a water soluble carrier.

An aqueous suspension contains the active ingredient in admixture with excipients suitable for the manufacture of an aqueous suspension. Such excipients are suspending agents, dispersants or humectants, and can be naturally occurring phospholipids. The aqueous suspension can also contain one or more preservatives, one or more colorants, one or more flavoring agents, and one or more sweeteners.

An oil suspension can be formulated by suspending the active ingredient in a vegetable oil, or in a mineral oil. The oil suspension can contain a thickener. The aforementioned sweeteners and flavoring agents can be added to provide a palatable preparation. These compositions can be preserved by adding an antioxidant.

The active ingredient and the dispersants or wetting agents, suspending agent or one or more preservatives can be prepared as a dispersible powder or granule suitable for the preparation of an aqueous suspension by adding water. Suitable dispersants or wetting agents and suspending agents are exemplified by those already mentioned above. Additional excipients, such as sweeteners, flavoring agents and colorants, can also be added. These compositions can be preserved by adding an antioxidant such as ascorbic acid.

The present pharmaceutical composition can also be in the form of an oil-in-water emulsion. The oil phase can be a vegetable oil, or a mineral oil, or mixture thereof. Suitable emulsifying agents can be naturally occurring phospholipids. Sweeteners can be used. Such formulations can also contain moderators, preservatives, colorants and antioxidants.

The pharmaceutical composition can be in the form of a sterile injectable aqueous solution. The acceptable vehicles and solvents that can be employed are water, Ringer's solution and isotonic sodium chloride solution. The sterile injectable preparation can also be a sterile injectable oil-in-water microemulsion in which the active ingredient is dissolved in the oil phase. The injectable solution or microemulsion can be introduced into an individual's bloodstream by local bolus injection. Alternatively, it can be advantageous to administer the solution or microemulsion in such a way as to maintain a constant circulating concentration of the present compound. In order to maintain such a constant concentration, a continuous intravenous delivery device can be utilized. An example of such a device is Deltec CADD-PLUS™ 5400 intravenous injection pump.

The pharmaceutical composition can be in the form of a sterile injectable aqueous or oily suspension for intratumoral, intramuscular and subcutaneous administration. Such a suspension can be formulated with suitable dispersants or wetting agents and suspending agents as described above according to known techniques. The sterile injectable preparation can also be a sterile injectable solution or suspension prepared in a nontoxic parenterally acceptable diluent or solvent. Moreover, sterile fixed oils can easily be used as a solvent or suspending medium, and fatty acids can also be used to prepare injections.

The present compound can be administered in the form of a suppository for rectal administration. These pharmaceutical compositions can be prepared by mixing the drug with a suitable non-irritating excipient that is solid at ordinary temperatures, but liquid in the rectum, thereby melting in the rectum to release the drug.

For buccal administration, the compositions can be formulated as tablets or lozenges by conventional means.

For intranasal administration or administration by inhalation, the active compounds of the present invention are conveniently delivered in the form of a solution or suspension released from a pump spray container that is squeezed or pumped by the patient, or as an aerosol spray released from a pressurized container or nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules or cartridges (for example, made from gelatin) for use in an inhaler or insufflator can be formulated containing a powder mix of the present invention and a suitable powder base such as lactose or starch.

It is well known to those skilled in the art that the dosage of a drug depends on a variety of factors, including but not limited to, the following factors: activity of the specific compound, age, weight, general health, behavior, diet of the patient, administration time, administration route, excretion rate, drug combination and the like. In addition, the best treatment, such as treatment mode, daily dose of the compound of formula (I) or the type of pharmaceutically acceptable salt thereof can be verified by traditional therapeutic regimens.

Any terms in the present application, unless specifically defined, will take the ordinary meanings as understood by a person of ordinary skill in the art.

Unless otherwise stated, the terms used in the specification and claims have the meanings described below.

“Alkyl” refers to a saturated aliphatic hydrocarbon group including C₁-C₂₀ straight chain and branched chain groups. Preferably an alkyl group is an alkyl having 1 to 12, sometimes preferably 1 to 6, sometimes more preferably 1 to 4, carbon atoms. Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. More preferably an alkyl group is a lower alkyl having 1 to 6 carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, preferably the substituent group(s) is one or more substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc., preferably C₂₋₂₀ alkenyl, more preferably C₂₋₁₂ alkenyl, and most preferably C₂₋₆ alkenyl. The alkenyl group can be substituted or unsubstituted. When substituted, the substituent group(s) is preferably one or more, sometimes preferably one to five, sometimes more preferably one to three, group(s) independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc., preferably C₂₋₂₀ alkynyl, more preferably C₂₋₁₂ alkynyl, and most preferably C₂₋₆ alkynyl. The alkynyl group can be substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkylene” refers to a saturated linear or branched aliphatic hydrocarbon group, wherein having 2 residues derived by removing two hydrogen atoms from the same carbon atom of the parent alkane or two different carbon atoms. The straight or branched chain group containing 1 to 20 carbon atoms, preferably has 1 to 12 carbon atoms, more preferably 1 to 6 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—) etc. The alkylene group can be substituted or unsubstituted.

When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkenylene” refers to an alkylene defined as above that has at least two carbon atoms and at least one carbon-carbon double bond, preferably C₂₋₂₀ alkenylene, more preferably C₂₋₁₂ alkenylene, and most preferably C₂₋₆ alkenylene. Non-limiting examples of alkenylene groups include, but are not limited to, —CH═CH—, —CH═CHCH₂—, —CH═CHCH₂CH₂—, —CH₂CH═CHCH₂— etc. The alkenylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkynylene” refers to an alkynyl defined as above that has at least two carbon atoms and at least one carbon-carbon triple bond, preferably C₂₋₂₀ alkynylene, more preferably C₂₋₁₂ alkynylene, and most preferably C₂₋₆ alkynylene. Non-limiting examples of alkenylene groups include, but are not limited to, —CH≡CH—, —CH≡CHCH₂—, —CH≡CHCH₂CH₂—, —CH₂CH≡CHCH₂— etc. The alkynylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Cycloalkyl” refers to a saturated and/or partially unsaturated monocyclic or polycyclic hydrocarbon group having 3 to 20 carbon atoms, preferably 3 to 12 carbon atoms, more preferably 3 to 10 carbon atoms, and most preferably 3 to 8 carbon atoms or 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more double bonds, but none of the rings has a completely conjugated pi-electron system. Preferably a spiro cycloalkyl is 6 to 14 membered, and more preferably 7 to 10 membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following substituents:

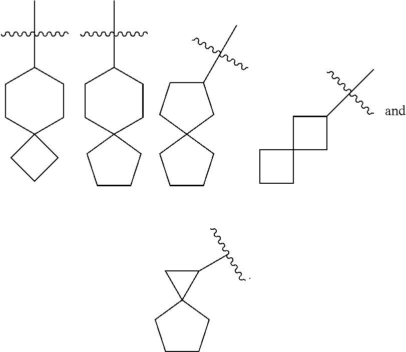

“Fused Cycloalkyl” refers to a 5 to 20 membered polycyclic hydrocarbon group, wherein each ring in the system shares an adjacent pair of carbon atoms with another ring, wherein one or more rings can contain one or more double bonds, but none of the rings has a completely conjugated pi-electron system. Preferably, a fused cycloalkyl group is 6 to 14 membered, more preferably 7 to 10 membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 5-membered/5-membered, or 5-membered/6-membered bicyclic fused cycloalkyl. Representative examples of fused cycloalkyls include, but are not limited to, the following substituents:

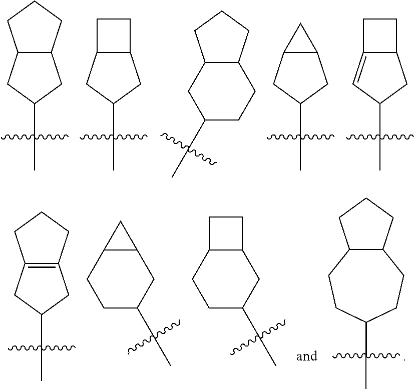

“Bridged Cycloalkyl” refers to a 5 to 20 membered polycyclic hydrocarbon group, wherein every two rings in the system share two disconnected carbon atoms. The rings can have one or more double bonds, but have no completely conjugated pi-electron system. Preferably, a bridged cycloalkyl is 6 to 14 membered, and more preferably 7 to 10 membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following substituents:

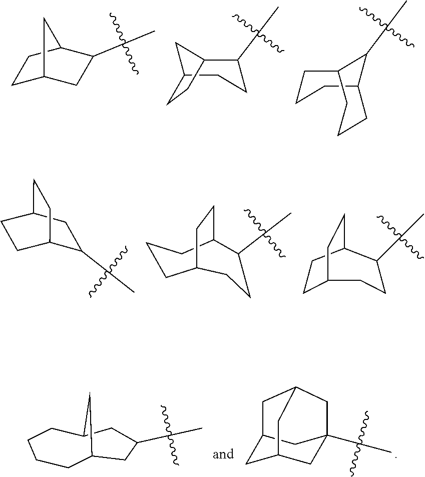

The cycloalkyl can be fused to the ring of an aryl, heteroaryl or heterocyclic alkyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, sometimes preferably one to five, sometimes more preferably one to three, substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Heterocyclyl” refers to a 3 to 20 membered saturated and/or partially unsaturated monocyclic or polycyclic hydrocarbon group having one or more, sometimes preferably one to five, sometimes more preferably one to three, heteroatoms selected from the group consisting of N, O, and S(O)ₘ (wherein m is 0,1, or 2) as ring atoms, but excluding —O—O—, —O—S— or —S—S— in the ring, the remaining ring atoms being C. Preferably, heterocyclyl is a 3 to 12 membered having 1 to 4 heteroatoms; more preferably a 3 to 10 membered having 1 to 3 heteroatoms; most preferably a 5 to 6 membered having 1 to 2 heteroatoms. Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more, sometimes preferably one to five, sometimes more preferably one to three, heteroatoms selected from the group consisting of N, O, and S(O)ₘ (wherein m is 0, 1 or 2) as ring atoms, the remaining ring atoms being C, wherein one or more rings can contain one or more double bonds, but none of the rings has a completely conjugated pi-electron system. Preferably a spiro heterocyclyl is 6 to 14 membered, and more preferably 7 to 10 membered. According to the number of common spiro atoms, spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following substituents:

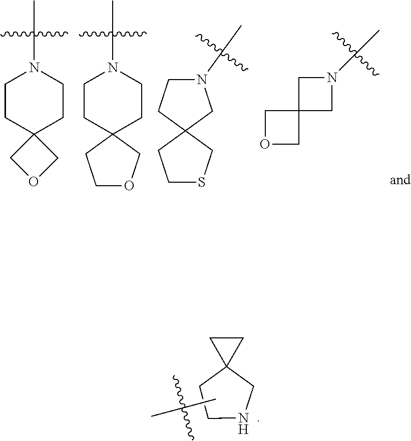

“Fused Heterocyclyl” refers to a 5 to 20 membered polycyclic heterocyclyl group, wherein each ring in the system shares an adjacent pair of carbon atoms with the other ring, wherein one or more rings can contain one or more double bonds, but none of the rings has a completely conjugated pi-electron system, and wherein said rings have one or more, sometimes preferably one to five, sometimes more preferably one to three, heteroatoms selected from the group consisting of N, O, and S(O)ₚ (wherein p is 0, 1, or 2) as ring atoms, the remaining ring atoms being C. Preferably a fused heterocyclyl is 6 to 14 membered, and more preferably 7 to 10 membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 5-membered/5-membered, or 5-membered/6-membered bicyclic fused heterocyclyl. Representative examples of fused heterocyclyl include, but are not limited to, the following substituents:

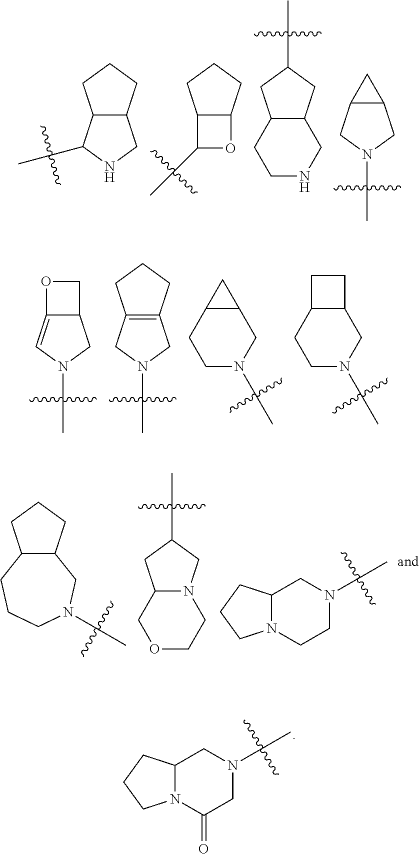

“Bridged Heterocyclyl” refers to a 5 to 14 membered polycyclic heterocyclic alkyl group, wherein every two rings in the system share two disconnected atoms, the rings can have one or more double bonds, but have no completely conjugated pi-electron system, and the rings have one or more heteroatoms selected from the group consisting of N, O, and S (O)ₘ (wherein m is 0, 1, or 2) as ring atoms, the remaining ring atoms being C. Preferably a bridged heterocyclyl is 6 to 14 membered, and more preferably 7 to 10 membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following substituents:

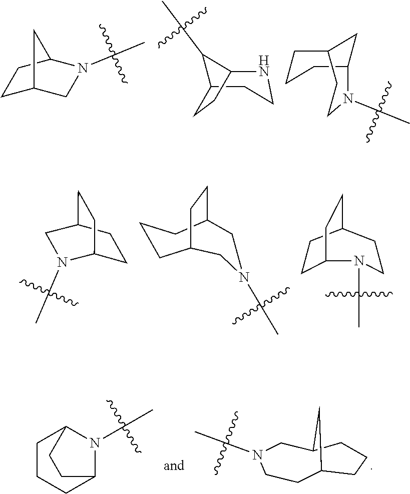

The ring of said heterocyclyl can be fused to the ring of an aryl, heteroaryl or cycloalkyl, wherein the ring bound to the parent structure is heterocyclyl. Representative examples include, but are not limited to the following substituents:

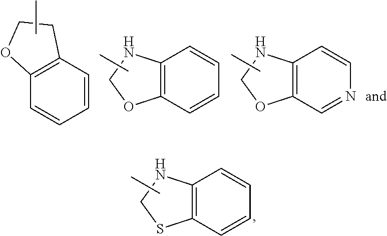

etc.

The heterocyclyl is optionally substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic alkylthio and —NR⁹R¹⁰.

“Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl can be fused to the ring of heteroaryl, heterocyclyl or cycloalkyl, wherein the ring bound to parent structure is aryl. Representative examples include, but are not limited to, the following substituents:

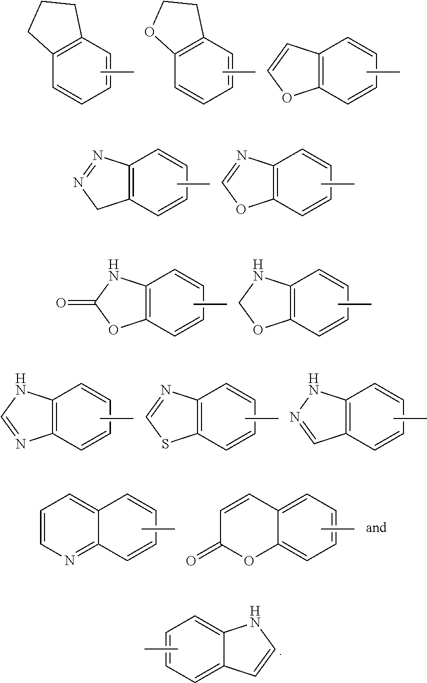

The aryl group can be substituted or unsubstituted. When substituted, the substituent group(s) is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic alkylthio and —NR⁹R¹⁰.

“Heteroaryl” refers to an aryl system having 1 to 4 heteroatoms selected from the group consisting of O, S and N as ring atoms and having 5 to 14 annular atoms. Preferably a heteroaryl is 5- to 10-membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl can be fused with the ring of an aryl, heterocyclyl or cycloalkyl, wherein the ring bound to parent structure is heteroaryl. Representative examples include, but are not limited to, the following substituents:

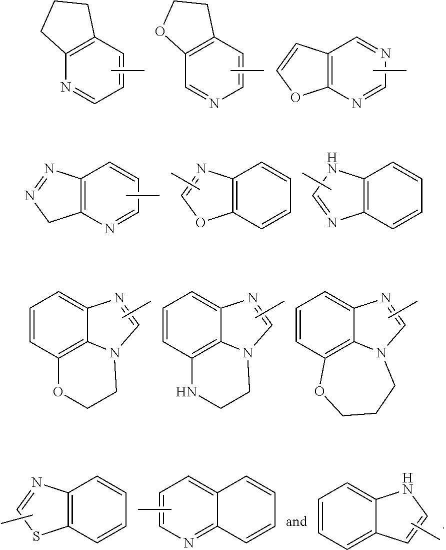

The heteroaryl group can be substituted or unsubstituted. When substituted, the substituent group(s) is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic alkylthio and —NR⁹R¹⁰.

“Alkoxy” refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxyl can be substituted or unsubstituted. When substituted, the substituent is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Bond” refers to a covalent bond using a sign of

“Hydroxyalkyl” refers to an alkyl group substituted by a hydroxy group, wherein alkyl is as defined above.

“Hydroxy” refers to an —OH group.

“Halogen” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(O)OH group.

“Alkoxycarbonyl” refers to a —C(O)O(alkyl) or (cycloalkyl) group, wherein the alkyl and cycloalkyl are defined as above.

“Optional” or “optionally” means that the event or circumstance described subsequently can, but need not, occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen atoms in the group, preferably up to 5, more preferably 1 to 3 hydrogen atoms, independently substituted with a corresponding number of substituents. It goes without saying that the substituents exist in their only possible chemical position. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present invention or physiologically/pharmaceutically acceptable salts or prodrugs thereof and other chemical components such as physiologically/pharmaceutically acceptable carriers and excipients. Suitable pharmaceutically acceptable excipients include, but are not limited to, diluents, lubricants, binders, disintegrants, fillers, glidants, granulating agents, coating agents, wetting agents, solvents, co-solvents, suspending agents, emulsifiers, sweeteners, flavoring agents, flavor masking agents, coloring agents, anti-caking agents, hemectants, chelating agents, plasticizers, viscosity increasing agents, antioxidants, preservatives, stabilizers, surfactants, and buffering agents.

The purpose of a pharmaceutical composition is to facilitate administration of a compound to an organism, which is conducive to the absorption of the active ingredient and thus displaying biological activity.

“Pharmaceutically acceptable salts” refer to salts of the compounds of the invention, such salts being safe and effective when used in a mammal and have corresponding biological activity. The salts can be prepared during the final isolation and purification of the compounds or separately by reacting a suitable nitrogen atom with a suitable acid. Acids commonly employed to form pharmaceutically acceptable salts include inorganic acids such as hydrochloric acid, hydrobromic acid, hydroiodic acid, sulfuric acid, phosphoric acid, hydrogen bisulfide as well as organic acids, such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, acetic acid acid, and related inorganic and organic acids.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include, but are not limited to, lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, and N-methylmorpholine.

As a person skilled in the art would understand, the compounds of formula (I) or Pharmaceutically acceptable salts thereof disclosed herein may exist in prodrug or solvate forms, which are all encompassed by the present invention.

The term “solvate,” as used herein, means a physical association of a compound of this invention with one or more, preferably one to three, solvent molecules, whether organic or inorganic. This physical association includes hydrogen bonding. In certain instances the solvate will be capable of isolation, for example when one or more, preferably one to three, solvent molecules are incorporated in the crystal lattice of the crystalline solid. Exemplary solvates include, but are not limited to, hydrates, ethanolates, methanolates, and isopropanolates. Methods of solvation are generally known in the art. “Prodrug” refers to compounds that can be transformed in vivo to yield the active parent compound under physiological conditions, such as through hydrolysis in blood. Common examples include, but are not limited to, ester and amide forms of a compound having an active form bearing a carboxylic acid moiety. Amides and esters of the compounds of the present invention may be prepared according to conventional methods. In particular, in the present invention, a prodrug may also be formed by acylation of an amino group or a nitrogen atom in a heterocyclyl ring structure, which acyl group can be hydrolyzed in vivo. Such acyl group includes, but is not limited to, a C₁-C₆ acyl, preferably C₁-C₄ acyl, and more preferably C₁-C₂ (formyl or acetyl) group, or benzoyl.

The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “therapeutically effective amount,” as used herein, refers to the total amount of each active component that is sufficient to show a meaningful patient benefit, e.g., a sustained reduction in viral load. When applied to an individual active ingredient, administered alone, the term refers to that ingredient alone. When applied to a combination, the term refers to combined amounts of the active ingredients that result in the therapeutic effect, whether administered in combination, serially, or simultaneously.

The term “subject” or “patient” includes both human and other mammals, especially domestic animals, for example, dogs, cats, horses, or the like, sometimes preferably a human.

As used herein, the singular forms “a”, “an”, and “the” include plural reference unless the context clearly dictates otherwise.

When the term “about” is applied to a parameter, such as pH, concentration, or the like, it indicates that the parameter can vary by ±10%, and some times more preferably within ±5%.

As would be understood by a person skilled in the art, when a parameter is not critical, a number is often given only for illustration purpose, instead of being limiting.

The term “treating” refers to: (i) inhibiting the disease, disorder, or condition, i.e., arresting its development; and (ii) relieving the disease, disorder, or condition, i.e., causing regression of the disease, disorder, and/or condition. In addition, the compounds of present invention may be used for their prophylactic effects in preventing a disease, disorder or condition from occurring in a subject that may be predisposed to the disease, disorder, and/or condition but has not yet been diagnosed as having it.

### Synthesis Methods

In order to complete the purpose of the invention, the present invention applies, but is not limited to, the following technical solution:

(A) A preparation process of a compound of formula (I), or a tautomer, mesomer, racemate, enantiomer, diastereomer, or mixture thereof, or a pharmaceutically acceptable salt thereof, the preparation process comprising a step of:

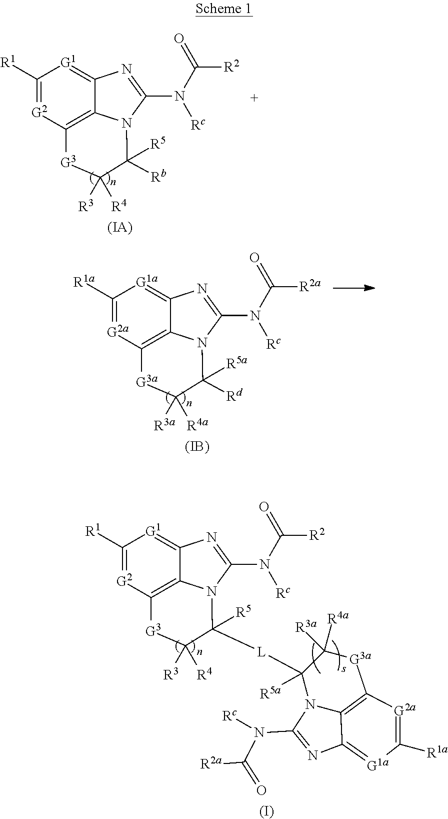

reacting a compound of formula (IA) with a compound of formula (IB) and the catalyst to obtain the compound of formula (I), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3; and

R¹ to R⁵, R^{1a} to R^{5a}, R^{c}, G¹ to G³, G^{1a} to G^{3a}, n and s are each as defined in formula (I).

The catalyst includes, but is not limited to, Hoveyda-Grubbs 2nd Gen Catalyst, Grubbs Catalyst (1^{st} gen, 2^{nd} gen, 3^{rd} gen, etc).

The reaction is preferably in solvent, wherein solvent used herein includes, but is not limited to, acetic acid, methanol, ethanol, toluene, tetrahydrofuran, dichloromethane, dimethylsulfoxide, 1,4-dioxane, water, N,N-dimethylformamide and the mixture thereof.

(B) A preparation process of a compound of formula (IM), or a tautomer, mesomer, racemate, enantiomer, diastereomer, or mixture thereof, or a pharmaceutically acceptable salt thereof, the preparation process comprising a step of:

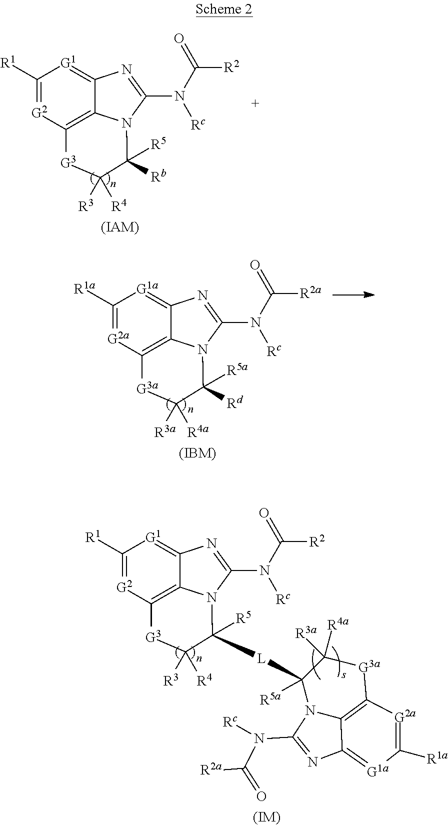

reacting a compound of formula (IAM) with a compound of formula (IBM) and the catalyst to obtain the compound of formula (IM), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3; and

R¹ to R⁵, R^{1a} to R^{5a}, R^{c}, G¹ to G³, G^{1a} to G^{3a}, n and s are each as defined in formula (IM).

The catalyst includes, but is not limited to, Hoveyda-Grubbs 2nd Gen Catalyst, Grubbs Catalyst (1^{st} gen, 2^{nd} gen, 3^{rd} gen, etc).

The reaction is preferably in solvent, wherein solvent used herein includes, but is not limited to, acetic acid, methanol, ethanol, toluene, tetrahydrofuran, dichloromethane, dimethylsulfoxide, 1,4-dioxane, water, N,N-dimethylformamide and the mixture thereof.

(C) A preparation process of a compound of formula (II), or a tautomer, mesomer, racemate, enantiomer, diastereomer, or mixture thereof, or a pharmaceutically acceptable salt thereof, the preparation process comprising a step of:

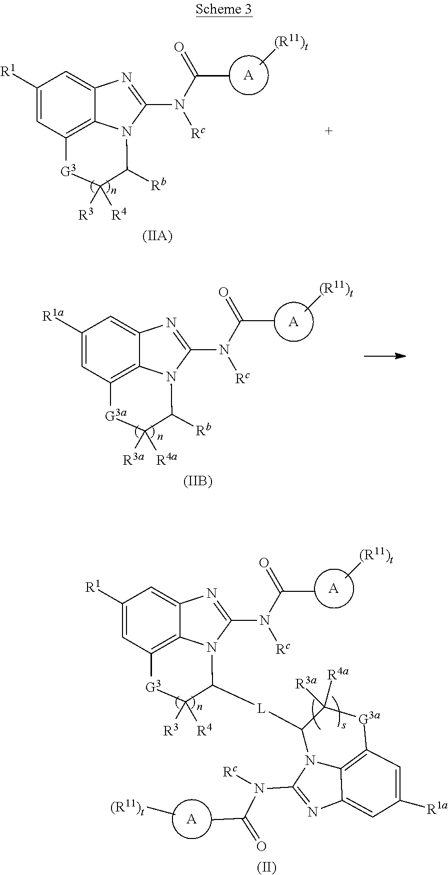

reacting a compound of formula (IIA) with a compound of formula (IIB) and the catalyst to obtain the compound of formula (II), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3; and

ring A, R¹, R^{1a}, R^{c}, R³, R⁴, R^{3a}, R^{4a}, R¹¹, G³, G^{3a}, t, n and s are each as defined in formula (II).

The catalyst includes, but is not limited to, Hoveyda-Grubbs 2nd Gen Catalyst, Grubbs Catalyst (1^{st} gen, 2^{nd} gen, 3^{rd} gen, etc).

The reaction is preferably in solvent, wherein solvent used herein includes, but is not limited to, acetic acid, methanol, ethanol, toluene, tetrahydrofuran, dichloromethane, dimethylsulfoxide, 1,4-dioxane, water, N,N-dimethylformamide and the mixture thereof.

(D) A preparation process of a compound of formula (IG), or a tautomer, mesomer, racemate, enantiomer, diastereomer, or mixture thereof, or a pharmaceutically acceptable salt thereof, the preparation process comprising a step of:

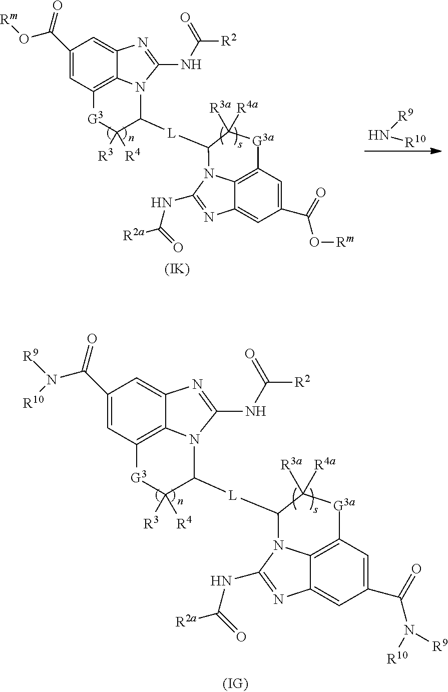

reacting a compound of formula (IK) with a compound of NHR⁹R¹⁰ under an alkaline condition to obtain the compound of formula (IG), wherein:

R^{m} is hydrogen or alkyl; and

R² to R⁴, R^{2a} to R^{4a}, G³, G^{3a}, R⁹, R¹⁰, n and s are each as defined in formula (IG).

The reagents that provide an alkaline condition include organic bases and inorganic bases. The organic bases include, but are not limited to triethylamine, N,N-diisopropylethylamine, n-butyllithium, lithium diisopropylamide, lithium bis(trimethylsilyl)amide, potassium acetate, sodium tert-butoxide and potassium tert-butoxide. The inorganic bases include, but are not limited to sodium hydride, potassium phosphate, sodium carbonate, potassium carbonate, cesium carbonate, sodium hydroxide and lithium hydroxide.

(E) A preparation process of a compound of formula (III), or a tautomer, mesomer, racemate, enantiomer, diastereomer, or mixture thereof, or a pharmaceutically acceptable salt thereof, the preparation process comprising a step of:

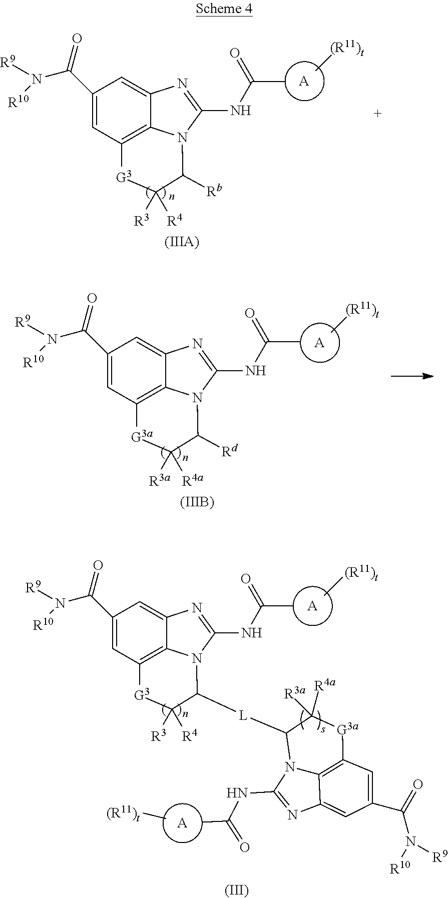

reacting a compound of formula (IIIA) with a compound of formula (IIB) and the catalyst to obtain the compound of formula (III), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3;

ring A, R³, R⁴, R^{3a}, R^{4a}, R⁹ to R¹¹, G³, G^{3a}, t, n and s are each as defined in formula (III).

The catalyst includes, but is not limited to, Hoveyda-Grubbs 2nd Gen Catalyst, Grubbs Catalyst (1^{st} gen, 2^{nd} gen, 3^{rd} gen, etc).

The reaction is preferably in solvent, wherein solvent used herein includes, but is not limited to, acetic acid, methanol, ethanol, toluene, tetrahydrofuran, dichloromethane, dimethylsulfoxide, 1,4-dioxane, water, N,N-dimethylformamide and the mixture thereof.

(F) A preparation process of a compound of formula (III), or a tautomer, mesomer, racemate, enantiomer, diastereomer, or mixture thereof, or a pharmaceutically acceptable salt thereof, the preparation process comprising a step of:

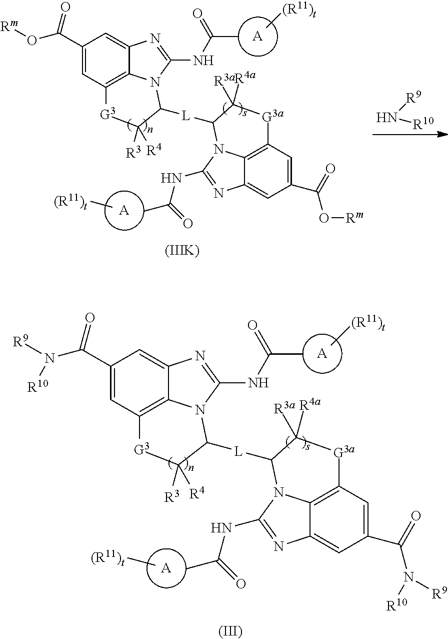

reacting a compound of formula (IIIK) with a compound of NHR⁹R¹⁰ under an alkaline condition to obtain the compound of formula (III), wherein:

R^{m} is hydrogen or alkyl; and

Ring A, R³, R⁴, R^{3a}, R^{4a}, G³, G^{3a}, R⁹˜R¹¹, t, n and s are each as defined in formula (III).

The reagents that provide an alkaline condition include organic bases and inorganic bases. The organic bases include, but are not limited to triethylamine, N,N-diisopropylethylamine, n-butyllithium, lithium diisopropylamide, lithium bis(trimethylsilyl)amide, potassium acetate, sodium tert-butoxide and potassium tert-butoxide. The inorganic bases include, but are not limited to sodium hydride, potassium phosphate, sodium carbonate, potassium carbonate, cesium carbonate, sodium hydroxide and lithium hydroxide.

(H) A preparation process of a compound of formula (IV), or a tautomer, mesomer, racemate, enantiomer, diastereomer, or mixture thereof, or a pharmaceutically acceptable salt thereof, the preparation process comprising a step of:

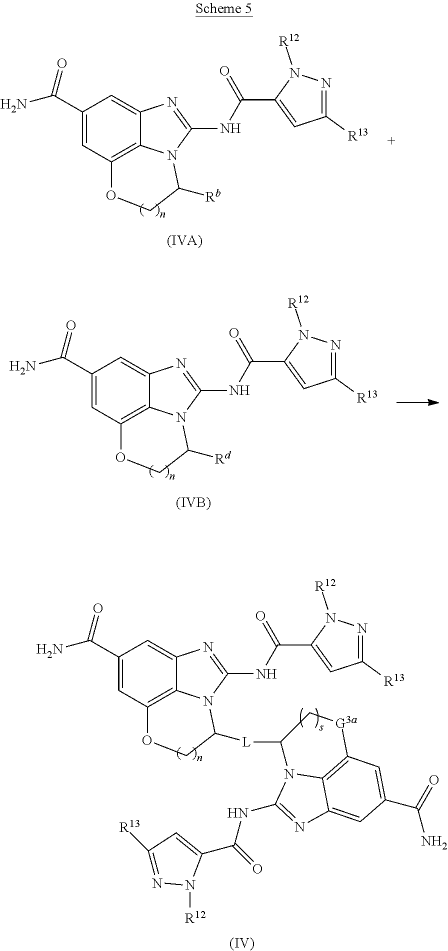

reacting a compound of formula (IVA) with a compound of formula (IVB) and the catalyst to obtain the compound of formula (IV), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3;

R¹², R¹³, G^{3a}, n and s are each as defined in formula (IV).

The catalyst includes, but is not limited to, Hoveyda-Grubbs 2nd Gen Catalyst, Grubbs Catalyst (1^{st} gen, 2^{nd} gen, 3^{rd} gen, etc).

The reaction is preferably in solvent, wherein solvent used herein includes, but is not limited to, acetic acid, methanol, ethanol, toluene, tetrahydrofuran, dichloromethane, dimethylsulfoxide, 1,4-dioxane, water, N,N-dimethylformamide and the mixture thereof.

(I) A preparation process of a compound of formula (IVM), or a tautomer, mesomer, racemate, enantiomer, diastereomer, or mixture thereof, or a pharmaceutically acceptable salt thereof, the preparation process comprising a step of:

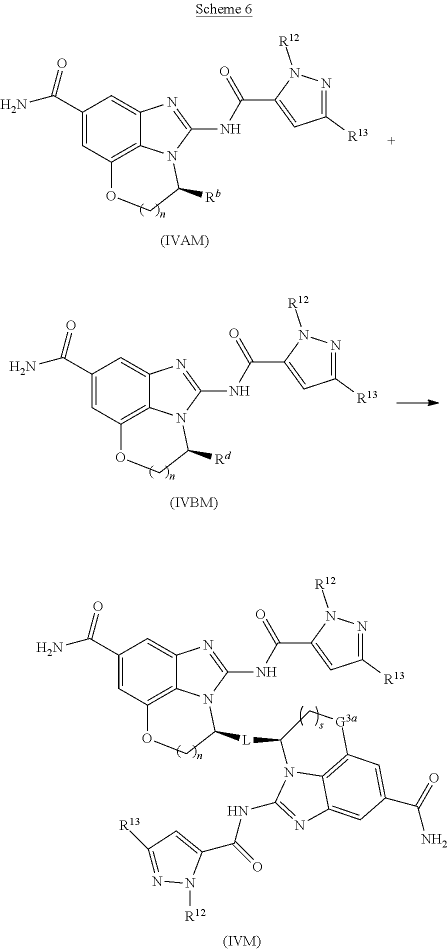

reacting a compound of formula (IVAM) with a compound of formula (IVBM) and the catalyst to obtain the compound of formula (IVM), wherein:

R^{b} is —(CH₂)ₚ₁—CH═CR^{e}R^{f};

R^{d} is —(CH₂)ₚ₂—CH═CR^{e}R^{f};

L is —(CH₂)ₚ₁—CH═CH—(CH₂)ₚ₂—;

R^{e} and R^{f} are identical or different, and each is independently selected from the group consisting of hydrogen and alkyl;

p¹ is 0, 1, 2 or 3;

p² is 0, 1, 2 or 3; and

R¹², R¹³, G^{3a}, n and s are each as defined in formula (IVM).

The catalyst includes, but is not limited to, Hoveyda-Grubbs 2nd Gen Catalyst, Grubbs Catalyst (1^{st} gen, 2^{nd} gen, 3^{rd} gen, etc).

The reaction is preferably in solvent, wherein solvent used herein includes, but is not limited to, acetic acid, methanol, ethanol, toluene, tetrahydrofuran, dichloromethane, dimethylsulfoxide, 1,4-dioxane, water, N,N-dimethylformamide and the mixture thereof.

### EXAMPLES

The following examples serve to illustrate the invention, but the examples should not be considered as limiting the scope of the invention. If specific conditions for the experimental method are not specified in the examples of the present invention, they are generally in accordance with conventional conditions or recommended conditions of the raw materials and the product manufacturer. The reagents without a specific source indicated are commercially available, conventional reagents.

The structure of each compound was identified by nuclear magnetic resonance (NMR) and/or mass spectrometry (MS). NMR chemical shifts (6) were given in 10⁻⁶ (ppm). NMR was determined by Bruker AVANCE-300, AVANCE-400 or AVANCE-500 machine. The solvents were deuterated-dimethyl sulfoxide (DMSO-d₆), deuterated-chloroform (CDCl₃) and deuterated-methanol (CD₃OD).

High performance liquid chromatography (HPLC) was determined on an Agilent 1200DAD high pressure liquid chromatography spectrometer (Sunfire C18 150×4.6 mm chromatographic column) and a Waters 2695-2996 high pressure liquid chromatography spectrometer (Gimini C18 150×4.6 mm chromatographic column).

Chiral High performance liquid chromatography (HPLC) is determined on LC-10A vp (Shimadzu) or SFC-analytical (Berger Instruments Inc.)

MS is determined by a SHIMADZU (ESI) liquid chromatography-mass spectrometer (manufacturer: Shimadzu, type: LC-20AD, LCMS-2020).

The average rates of kinase inhibition, and the IC₅₀ values were determined by Microplate reader (BMG company, Germany).

The thin-layer silica gel plates used in thin-layer chromatography were Yantai Huanghai HSGF254 or Qingdao GF254 silica gel plate. The dimension of the plates used in TLC was 0.15 mm to 0.2 mm, and the dimension of the plates used in thin-layer chromatography for product purification was 0.4 mm to 0.5 mm.

Column chromatography generally used Yantai Huanghai 200 to 300 mesh silica gel as carrier.

The known starting material of the invention can be prepared by the conventional synthesis method in the prior art, or can be purchased from ABCR GmbH & Co. KG, Acros Organics, Aldrich Chemical Company, Accela ChemBio Inc or Dari chemical Company, etc.

Unless otherwise stated in the examples, the following reactions were placed under argon atmosphere or nitrogen atmosphere.

The term “argon atmosphere” or “nitrogen atmosphere” means that a reaction flask was equipped with a balloon having 1 L of argon or nitrogen.

The term “hydrogen atmosphere” means that a reaction flask was equipped with a balloon having 1 L of hydrogen.

High pressure hydrogenation reactions were performed with a Parr 3916EKX hydrogenation apparatus and clear blue QL-500 hydrogen generator or HC2-SS hydrogenation apparatus.

In hydrogenation reactions, the reaction system was generally vacuumed and filled with hydrogen, and the above operation was repeated three times.

Microwave reactions were performed with a CEM Discover-S 908860 microwave reactor.

Unless otherwise stated in the examples, the solution used in following reactions refers to an aqueous solution.

Unless otherwise stated in the examples, the reaction temperature in the following reactions was room temperature.

Room temperature was the most proper reaction temperature, which was 20° C. to 30° C.

The reaction process is monitored by thin layer chromatography (TLC), and the developing solvent system includes: A: dichloromethane and methanol, B: hexane and ethyl acetate. The ratio of the volume of the solvent can be adjusted according to the polarity of the compounds. The elution system for purification of the compounds by column chromatography, thin layer chromatography and CombiFlash flash rapid preparation instrument includes: A: dichloromethane and methanol, B: hexane and ethyl acetate. The ratio of the volume of the solvent can be adjusted according to the polarity of the compounds, and sometimes a small amount of basic reagent such as ammonia or acidic reagent such as acetic acid can be added.

Final compounds are purified by Shimadzu (LC-20AD, SPD20A) Prepative HPLC (Phenomenex Gemini-NX 5 uM C18 21.2×100 mm column) with water/MeOH or water/CH₃CN elution systems with optional additives, such as HCOOH, TFA.

The following abbreviations are used:

Hoveyda-Grubbs 2nd Gen Catalyst is (1,3-Bis-(2,4,6-trimethylphenyl)-2-imidazolidinylidene)dichloro(o-isopropoxyphenylmethylene)ruthenium (Sigma-Aldrich),

Grubb's (II) catalyst is (1,3-Bis(2,4,6-trimethylphenyl)-2-imidazolidinylidene)dichloro(phenylmethylene)(tricyclohexylphosphine)ruthenium,

TEA is triethylamine,

HATU is 1-[Bis(dimethylamino)methylene]-1H-1,2,3-triazolo[4,5-b]pyridinium 3-oxide hexafluorophosphate

HBTU is O-(Benzotriazol-1-yl)-N,N,N′,N′-tetramethyluronium hexafluorophosphate,

DCM is dichloromathene,

DMF is N,N-dimethylformamide,

DMSO is dimethyl sulfoxide,

DEAD is diethyl azodiformate,

DIAD is diisopropyl azodicarboxylate,

EDCI is N-Ethyl-N′-(3-dimethylaminopropyl)carbodiimide hydrochloride,

EtOAc is ethyl acetate,

Prep HPLC is Prepative High performance liquid chromatography.

NMR is proton nuclear magnetic resonance, and

MS is mass spectroscopy with (+) referring to the positive mode which generally gives a M+1 (or M+H) absorption where M=the molecular mass.

### Examples 1 and 2

### (3S,3″S)-3,3″-((E)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxamide) 1

### (3S,3″S)-3,3″-((Z)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxamide) 2

### Method A

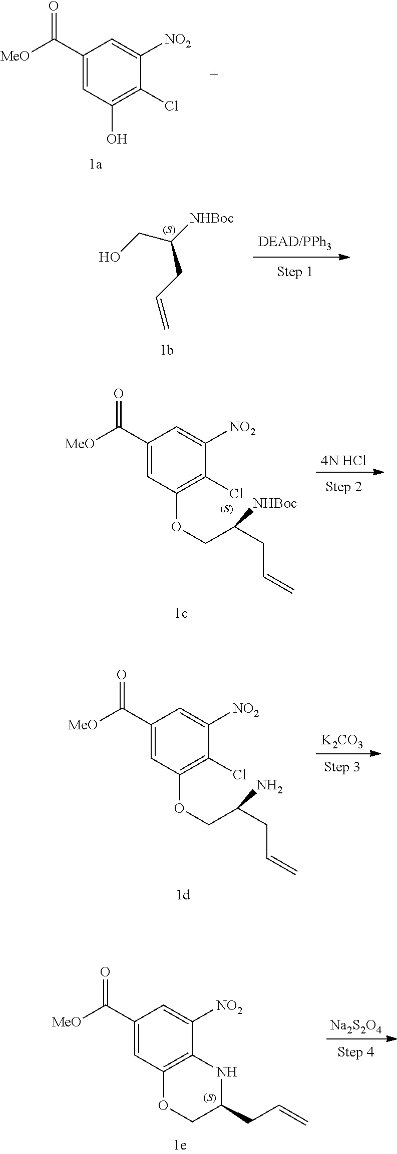

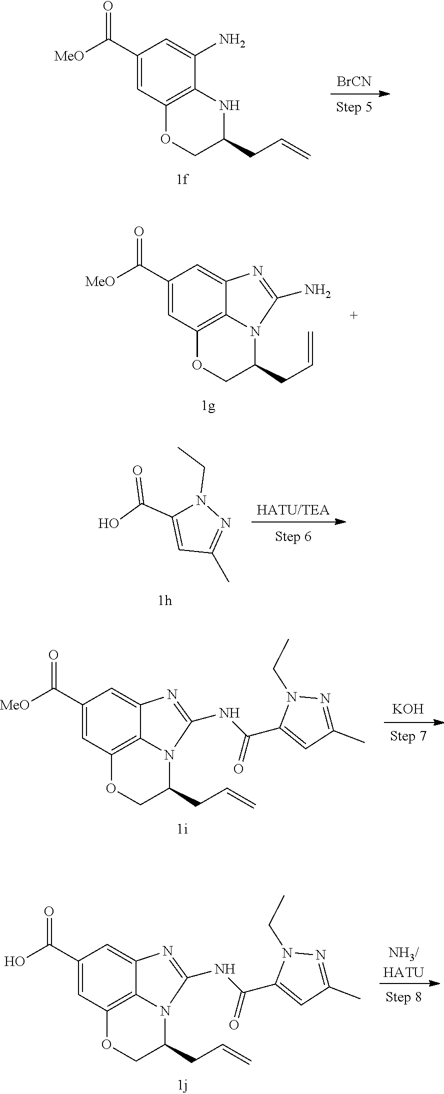

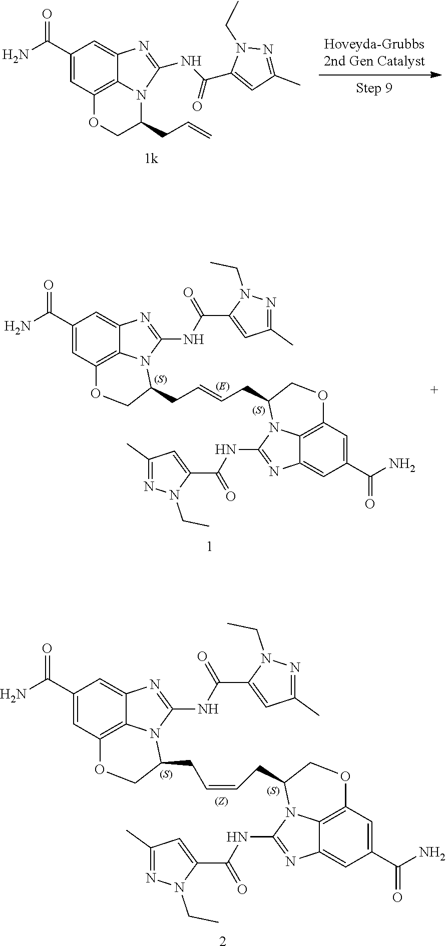

### Step 1 (S)-methyl 3-((2-((tert-butoxycarbonyl)amino)pent-4-en-1-yl)oxy)-4-chloro-5-nitrobenzoate 1c

To the methylene chloride solution (˜15 mL) of Methyl 4-chloro-3-hydroxy-5-nitrobenzoate 1a (205 mg, 0.885 mmol, 1.0 eq) and (S)-tert-butyl (1-hydroxypent-4-en-2-yl)carbamate 1b (250 mg 1.24 mmol, 1.4 eq), was added PPh₃ (350 mg, 1.33 mmol, 1.5 eq) followed with DEAD (210 uL, 1.33 mmol, 1.5 eq), the reaction mixture was stirred at room temperature for overnight. The Mixture was concentrated under vacuum and purified by silica gel column (40 g ISCO cartridge with 20% EtOAc in Hexane) to give title compound 1c (S)-methyl 3-((2-((tert-butoxycarbonyl)amino)pent-4-en-1-yl)oxy)-4-chloro-5-nitrobenzoate (280 mg, 76%).

### Step 2 (S)-methyl 3-((2-aminopent-4-en-1-yl)oxy)-4-chloro-5-nitrobenzoate 1d

To the methylene chloride solution (˜10 mL) of (S)-methyl 3-((2-((tert-butoxycarbonyl)amino)pent-4-en-1-yl)oxy)-4-chloro-5-nitrobenzoate 1c (280 mg, 0.675 mmol, 1 eq) was added 4 N HCl in Dioxane (6 mL, 24 mmol, 35.6 eq), the reaction mixture was stirred at room temperature for 3 hours. The volatile was evaporated under vacuum to give title compound 1d (S)-methyl 3-((2-aminopent-4-en-1-yl)oxy)-4-chloro-5-nitrobenzoate (230 mg, 97%). MS m/z (ESI): 315 [M+1].

### Step 3 (S)-methyl 3-allyl-5-nitro-3,4-dihydro-2H-benzo[b][1,4]oxazine-7-carboxylate 1e

To the DMSO solution (˜7 mL) of (S)-methyl 3-((2-aminopent-4-en-1-yl)oxy)-4-chloro-5-nitrobenzoate 1d (230 mg, 0.655 mmol, 1 eq) was added triethylamine (140 uL, 0.98 mmol, 1.5 eq) followed with K₂CO₃ (270 mg, 1.96 mmol, 3 eq), the reaction mixture was heated at 100° C. for 3 hours. The mixture was cooled down to room temperature, water (˜30 mL) was added. The precipitates were collected by filtration to give title compound 1e (S)-methyl 3-allyl-5-nitro-3,4-dihydro-2H-benzo[b][1,4]oxazine-7-carboxylate (120 mg, 66%).

MS m/z (ESI): 279 [M+1].

### Step 4 (S)-methyl 3-allyl-5-amino-3,4-dihydro-2H-benzo[b][1,4]oxazine-7-carboxylate 1f

To the MeOH solution (˜15 mL) of (S)-methyl 3-allyl-5-nitro-3,4-dihydro-2H-benzo[b][1,4]oxazine-7-carboxylate 1e (120 mg, 0.431 mmol, 1 eq) was added Na₂S₂O₄ (751 mg, 4.31 mmol, 10 eq) in 5 mL water, followed with conc. NH₄OH (0.78 mL, 10.8 mmol, 25 eq), the reaction mixture was stirred at room temperature for 1 hour. The mixture was diluted with water (20 mL), extracted with EtOAc (30 mL×3). Organic layer was combined, washed with brine (20 mL xl), dried over Na₂SO₄, filtered and the filtrated was concentrated under vacuum to give crude title compound if (S)-methyl 3-allyl-5-amino-3,4-dihydro-2H-benzo[b][1,4]oxazine-7-carboxylate (118 mg), which was used in the next step without further purification. MS m/z (ESI): 249 [M+1].

### Step 5 (S)-methyl 3-allyl-2-amino-3,4-dihydro-5-oxa-1,2a-diazaacenaphthylene-7-carboxylate 1g

To the MeOH solution (˜20 mL) of (S)-methyl 3-allyl-5-amino-3,4-dihydro-2H-benzo[b][1,4]oxazine-7-carboxylate 1f (120 mg, 0.475 mmol, 1 eq) was added BrCN (76 mg, 0.713 mmol, 1.5 eq), the reaction mixture was stirred at room temperature for overnight. The mixture was concentrated under vacuum to give crude title compound 1g (S)-methyl 3-allyl-2-amino-3,4-dihydro-5-oxa-1,2a-diazaacenaphthylene-7-carboxylate (160 mg), which was used in the next step without further purification. MS m/z (ESI): 274 [M+1].

### Step 6 (S)-methyl 3-allyl-2-(1-ethyl-3-methyl-1H-pyrazole-5-carboxamido)-3,4-dihydro-5-oxa-1,2a-diazaacenaphthylene-7-carboxylate 1i

To the DCM (˜15 mL) and DMF (˜3 mL) solution of (S)-methyl 3-allyl-2-amino-3,4-dihydro-5-oxa-1,2a-diazaacenaphthylene-7-carboxylate 1g (160 mg, 0.463 mmol, 1 eq) was added 1-ethyl-3-methyl-1H-pyrazole-5-carboxylic acid 1h (107 mg, 0.694 mmol, 1.5 eq), HATU (264 mg, 0.694 mmol, 1.5 eq) and TEA (325 uL, 2.32 mmol, 5 eq) the reaction mixture was stirred at room temperature for overnight. LC-MS showed ˜25% starting material 1g exist, another 0.5 eq (36 mg, 0.232 mmol) 1-ethyl-3-methyl-1H-pyrazole-5-carboxylic acid 1h and HATU (88 mg, 0.232 mmol, 0.5 eq) were added and the mixture was stirred at room temperature for overnight. The mixture was diluted with DCM (30 mL), washed with water (10 mL×1), dried over Na₂SO₄, filtered and the filtrated was concentrated under vacuum. The residue was purified by silica gel column (24 g ISCO cartridge with 10% EtOH and 30% EtOAc in Hexane) to give title compound 1i (S)-methyl 3-allyl-2-(1-ethyl-3-methyl-1H-pyrazole-5-carboxamido)-3,4-dihydro-5-oxa-1,2a-diazaacenaphthylene-7-carboxylate (150 mg, 80%). MS m/z (ESI): 410 [M+1].

### Step 7 (S)-3-allyl-2-(1-ethyl-3-methyl-1H-pyrazole-5-carboxamido)-3,4-dihydro-5-oxa-1,2a-diazaacenaphthylene-7-carboxylic acid 1j

To the MeOH solution (1.5 mL) of (S)-methyl 3-allyl-2-(1-ethyl-3-methyl-1H-pyrazole-5-carboxamido)-3,4-dihydro-5-oxa-1,2a-diazaacenaphthylene-7-carboxylate 1i (10 mg, 0.024 mmol, 1 eq) was added 5 N KOH aqueous solution (1.5 mL), the reaction mixture was stirred at room temperature for overnight. The mixture was acidified by 6 N HCl to pH<5, diluted with water (10 mL), extracted with EtOAc (10 mL×3). Organic layer was combined, washed with brine (10 mL×1), dried over Na₂SO₄, filtered and the filtrate was concentrated under vacuum to give crude title compound 1j (S)-3-allyl-2-(1-ethyl-3-methyl-1H-pyrazole-5-carboxamido)-3,4-dihydro-5-oxa-1,2a-diazaacenaphthylene-7-carboxylic acid (12 mg, which was used in the next step without further purification. MS m/z (ESI): 396 [M+1].

### Step 8 (S)-3-allyl-2-(1-ethyl-3-methyl-1H-pyrazole-5-carboxamido)-3,4-dihydro-5-oxa-1,2a-diazaacenaphthylene-7-carboxamide 1k

To the DMF (˜1 mL) solution of (S)-3-allyl-2-(1-ethyl-3-methyl-1H-pyrazole-5-carboxamido)-3,4-dihydro-5-oxa-1,2a-diazaacenaphthylene-7-carboxylic acid 1j (12 mg, 0.03 mmol, 1 eq) was added 7 N ammonia in MeOH (50 uL, 0.35 mmol, 12 eq), HATU (17.3 mg, 0.046 mmol, 1.5 eq) and TEA (12.6 uL, 0.09 mmol, 3 eq) the reaction mixture was stirred at room temperature for 2 hours. The mixture was purified by reverse phase HPLC, eluted with AcCN/H₂O/HCOOH to give title compound 1k (S)-3-allyl-2-(1-ethyl-3-methyl-1H-pyrazole-5-carboxamido)-3,4-dihydro-5-oxa-1,2a-diazaacenaphthylene-7-carboxamide (2.2 mg, 23%).

MS m/z (ESI): 395 [M+1].

### Step 9

To a solution of 1k (42 mg, 0.11 mmol, 1 eq) in DCM (1.5 mL)/MeOH (1.5 mL) was added p-toluenesulfonic acid monohydrate (27 mg in 1.5 mL MeOH, 0.14 mmol, 1.27 eq). The resulting mixture was stirred at room temperature for 15 min and removed solvents. The residue was dissolved in DCM (2 mL) and transferred to a seal tube. Hoveyda-Grubbs 2nd Gen Catalyst (15.5 mg, 0.025 mmol, 0.23 eq) was added. The seal tube was degassed with N₂ and stirred at 80° C. for 18 h. Small amount of MeOH was added, after 5 min, the solvents were removed under vacuum, the residue was purified by reverse phase HPLC, eluted with AcCN/H₂O/TFA to give title compound 1 (7 mg) and 2 (11 mg).

Example 1 (Shorter retention time on reverse phase HPLC) (3S,3″S)-3,3″-((E)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxamide) MS m/z (ESI): 761 [M+1]; ¹H NMR (300 MHz, Methanol-d₄) δ 7.60 (d, 2H), 7.31 (d, 2H), 6.62 (s, 2H), 5.63-5.69 (m, 2H), 4.73-4.49 (m, 6H), 4.44 (d, 2H), 4.24-4.13 (m, 2H), 2.54-2.64 (m, 4H), 2.21 (s, 6H), 1.35 (t, 6H).

Example 2 (Longer retention time on reverse phase HPLC) (3S,3″S)-3,3″-((Z)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxamide) MS m/z (ESI): 761 [M+1]; ¹H NMR (300 MHz, Methanol-d₄) δ 7.52 (d, 2H), 7.28 (d, 2H), 6.45 (s, 2H), 5.85-5.91 (m, 2H), 4.67-4.52 (m, 6H), 4.21 (m, 2H), 2.89-2.99 (m, 2H), 2.34 (s, 2H), 1.88 (s, 6H), 1.43 (t, 6H), 1.31 (s, 2H).

### Example 3

### (3S,3″S)-3,3″-((E)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylic Acid) 3

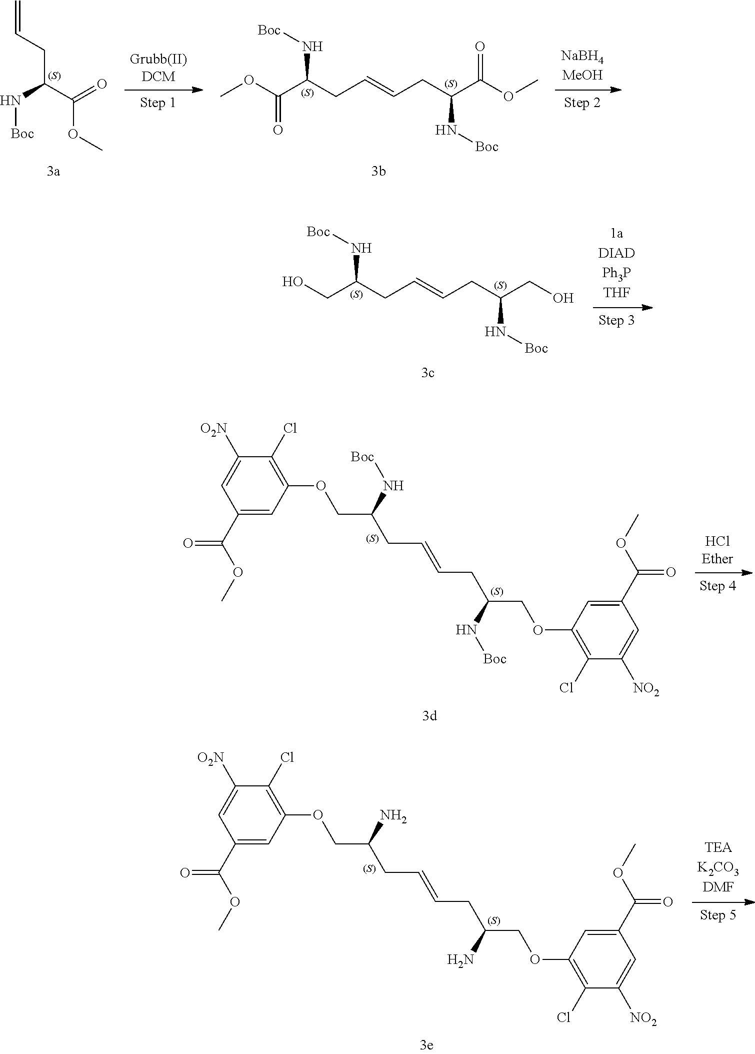

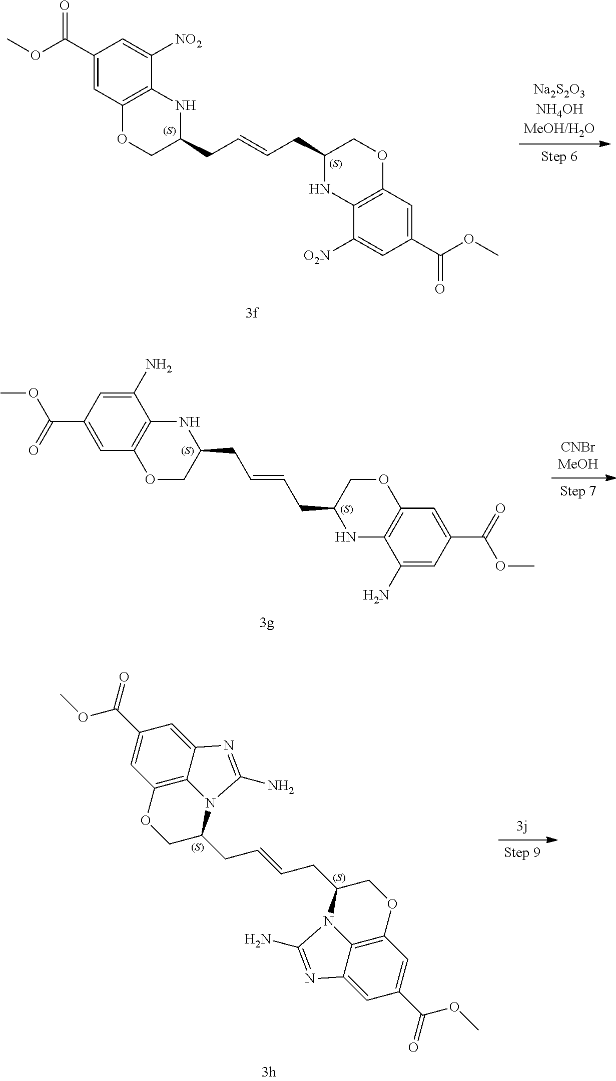

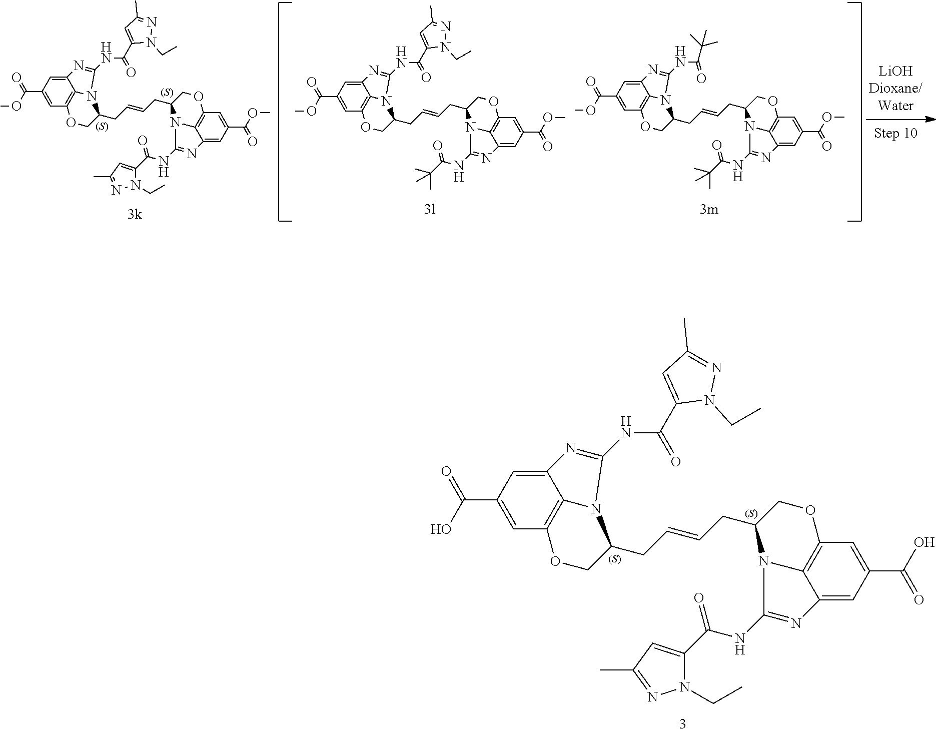

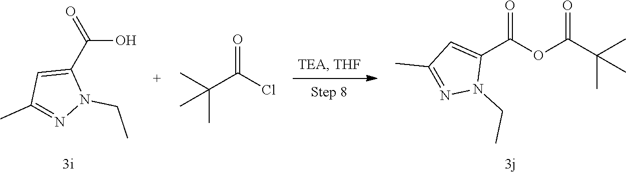

### Step 1 (2S, 7S,E)-dimethyl 2,7-bis((tert-butoxycarbonyl)amino)oct-4-enedioate 3b

To a solution of the (S)-methyl 2-((tert-butoxycarbonyl)amino)pent-4-enoate 3a (25 g, 109 mmol) in dichloromethane (500 mL) was added Grubb's (II) catalyst (1 g). After addition, the reaction was refluxed for 16 hours, TLC showed that the monomer was completely converted. It was concentrated, and purified on a silica gel column, eluting with 40% ethyl acetate in hexanes, to get (2S,7S,E)-dimethyl 2,7-bis((tert-butoxycarbonyl)amino)oct-4-enedioate 3b (22.1 g, 94% yield). MS m/z (ESI): 431[M+1]. ¹H NMR (400 MHz, DMSO-d₆): δ 5.43 (bs, 2H), 5.12 (bs, 2H), 4.37 (m, 2H), 3.77 (s, 6H), 2.49, m, 4H), 1.47 (s, 18H). A minor isomer (cis) was also isolated (More polar) (2.1 g, 5% yield). ¹H NMR (400 MHz, DMSO-d₆): δ 5.48 (m, 2H), 5.18 (m, 2H), 4.38 (m, 2H), 3.77 (s, 6H), 2.49 (m, 4H), 1.47 (s, 18H).

### Step 2 di-tert-butyl ((2S,7S,E)-1,8-dihydroxyoct-4-ene-2,7-diyl)dicarbamate 3c

To a solution of (2S,7S,E)-dimethyl 2,7-bis((tert-butoxycarbonyl)amino)oct-4-enedioate 3b (24 g, 55.74 mmol) in anhydrous MeOH (300 mL) was added sodium borohydride (8.4 g, 223 mmol) slowly at 0° C. After addition, the reaction was stirred at ambient temperature for 16 hours. Acetic acid was added to adjust the pH value to about 5. It was concentrated, and the residue was dissolved in DCM (200 mL). It was filtered. The filtrate was concentrated and purified on a silica gel column, eluting with 60% ethyl acetate in hexanes, to get the title compound di-tert-butyl ((2S,7S,E)-1,8-dihydroxyoct-4-ene-2,7-diyl)dicarbamate 3c (20.1 g, 96.3% yield). MS m/z (ESI): 375 [M+1]; ¹H NMR (400 MHz, DMSO-d₆): δ 5.53 (m, 2H), 3.54 (m, 2H), 3.49 (m, 4H), 2.32-2.11 (m, 4H), 1.46 (s, 18H).

### Step 3 dimethyl 5,5′-(((2S,7S,E)-2,7-bis((tert-butoxycarbonyl)amino)oct-4-ene-1,8-diyl)bis(oxy))bis(4-chloro-3-nitrobenzoate) 3d

To a solution of DIAD (10.8 g, 53.4 mmol) in tetrahydrofuran (50 mL) was added triphenyl phosphene (14.0 g, 53.4 mmol) at 0° C. It was stirred at 0° C. for 10 minutes before methyl 4-chloro-3-hydroxy-5-nitrobenzoate 1a (3.1 g, 13.4 mmol) was added, followed by the di-tert-butyl ((2S,7S,E)-1,8-dihydroxyoct-4-ene-2,7-diyl)dicarbamate 3c (5.0 g, 13.4 mmol). After addition, the reaction was stirred at ambient temperature for 10 hours. It was concentrated. The crude stuff was purified on a silica gel column, eluting with 60% ethyl acetate in hexanes, to get the title compound, dimethyl 5,5′-(((2S,7S,E)-2,7-bis((tert-butoxycarbonyl)amino)oct-4-ene-1,8-diyl)bis(oxy))bis(4-chloro-3-nitrobenzoate) 3d (9.0 g, 84.1%). MS m/z (ESI): 801 [M+1]; ¹H NMR (500 MHz, CDCl₃): δ 8.05 (d, 0.5 Hz, 2H), 7.84 (d, 0.5 Hz, 2H), 5.67 (m, 2 H), 4.20 (m, 4H), 4.13 (m, 4H), 3.98 (m, 2H), 3.96 (s, 6H), 2.50 (m, 2H), 1.45 (s, 18H).

### Step 4 dimethyl 5,5′-(((2S,7S,E)-2,7-diaminooct-4-ene-1,8-diyl)bis(oxy))bis(4-chloro-3-nitrobenzoate) 3e

To a solution of dimethyl 5,5′-(((2S,7S,E)-2,7-bis((tert-butoxycarbonyl)amino)oct-4-ene-1,8-diyl)bis(oxy))bis(4-chloro-3-nitrobenzoate) 3d (9.0 g, 11.2 mmol) in dichloromethane (100 mL) was added trifluoroacetic acid (25 mL) at room temperature. Then, the reaction was stirred at ambient temperature for 14 hours. It was concentrated and washed with dichloromethane and ether to get the title compound dimethyl 5,5′-(((2S,7S,E)-2,7-diaminooct-4-ene-1,8-diyl)bis(oxy))bis(4-chloro-3-nitrobenzoate) 3e (5.9 g, 88%). This was used for the next step without further purification. MS m/z (ESI): 601 [M+1].

### Step 5 (3S,3′S)-dimethyl 3,3′-((E)-but-2-ene-1,4-diyl)bis(5-nitro-3,4-dihydro-2H-benzo[b][1,4]oxazine-7-carboxylate) 3f

To a solution of the dimethyl 5,5′-(((2S,7S,E)-2,7-diaminooct-4-ene-1,8-diyl)bis(oxy))bis(4-chloro-3-nitrobenzoate) 3e, from the previous step, in N,N-dimethylformamide (20 mL) was added triethylamine (4.6 g, 45 mmol) and potassium carbonate (9.3 g, 67.4 mmol). After addition, the reaction was stirred at 100° C. for 2 hours. LCMS showed that the start material was completely converted. It was concentrated and absorbed onto silica gel. It was eluted with 20% ethyl acetate in dichloromethane to get the title compound (3S,3′S)-dimethyl 3,3′-((E)-but-2-ene-1,4-diyl)bis(5-nitro-3,4-dihydro-2H-benzo[b][1,4]oxazine-7-carboxylate) 3f (7.00 g, 98% yield). MS m/z (ESI): 529 [M+1],

### Step 6 (3S,3′S)-dimethyl 3,3′-((E)-but-2-ene-1,4-diyl)bis(5-amino-3,4-dihydro-2H-benzo[b][1,4]oxazine-7-carboxylate) 3g

To a solution of (3S,3′S)-dimethyl 3,3′-((E)-but-2-ene-1,4-diyl)bis(5-nitro-3,4-dihydro-2H-benzo[b][1,4]oxazine-7-carboxylate) 3f (3.7 g, 6.2 mmol) in methanol (150 mL) was added sodium bisulfite (30.5 g, 175 mmol) in water (40 mL), followed by concentrated ammonium hydroxide (40 mL). The reaction was stirred at ambient temperature for 4 hours. LCMS showed that the reaction was done. It was extracted with ethyl acetate for several times. The combined organic layer was concentrated, dry loaded to a silica gel column, and was eluted with 20% ethyl acetate in dichloromethane to get the title compound (3S,3′S)-dimethyl 3,3′-((E)-but-2-ene-1,4-diyl)bis(5-amino-3,4-dihydro-2H-benzo[b][1,4]oxazine-7-carboxylate) 3g (2.0 g, 69%). MS m/z (ESI): 469 [M+1]; ¹H NMR (400 MHz, DMSO-d₆): δ 6.89 (s, 2H), 6.69 (s, 2H), 5.64 (m, 2H), 5.43 (m, 2H), 4.81 (m, 4H), 4.10 (m, 2H), 3.75 (s, 4H). 3.72 (s, 6H).

### Step 7 (3S,3′S)-dimethyl 3,3′-((E)-but-2-ene-1,4-diyl)bis(2-amino-3,4-dihydro-5-oxa-1,2a-diazaacenaphthylene-7-carboxylate) 3h

To a suspension of (3S,3′S)-dimethyl 3,3′-((E)-but-2-ene-1,4-diyl)bis(5-amino-3,4-dihydro-2H-benzo[b][1,4]oxazine-7-carboxylate) 3g (2.0 g, 4.27 mmol) in anhydrous methanol (100 mL) was added cyanogen bromide (1.81 g, 17.1 mmol). After addition, the reaction was stirred at ambient temperature for 16 hours to get a clear solution. LCMS showed that the reaction was completed. It was concentrated. The crude product was purified on a silica gel column, eluting with 20% methanol (containing 7 N ammonia) in dichloromethane, to get (3S,3′S)-dimethyl 3,3′-((E)-but-2-ene-1,4-diyl)bis(2-amino-3,4-dihydro-5-oxa-1,2a-diazaacenaphthylene-7-carboxylate) 3h (1.95 g, 88.1% yield). MS m/z (ESI): 519 [M+1]. ¹H NMR (400 MHz Methanol-d₄): δ 7.59 (s, 2H), 7.22 (s, 2H), 5.55 (s, 2H), 4.61 (s, 2H), 4.45 (s, 2H), 4.31 (d, J=12 Hz, 2H), 4.07 (d, 12 Hz, 2H), 3.90 (s, 6H), 3.37 (s, 2H), 2.44 (broad s, 4H). ¹³C NMR (400 MHz, Methanol-d₄): δ 168.3, 154.3, 140.9, 140.7, 129.2, 124.7, 123.6, 110.1, 106.2, 68.0, 51.6, 51.1, 34.5.

### Step 8 1-ethyl-3-methyl-1H-pyrazole-5-carboxylic Pivalic Anhydride 3j

To a solution of the 1-ethyl-3-methyl-1H-pyrazole-5-carboxylic acid 3i (4.7 g, 30.5 mmol) and triethylamine (3.5 g, 34.8 mmol) in THF (50 mL), at 0° C., was added pivaloyl chloride (3.5 g, 29 mmol). After addition, the reaction was stirred at ambient temperature for 1 hour. It was filtered, and the filtrate was concentrated to get 1-ethyl-3-methyl-1H-pyrazole-5-carboxylic pivalic anhydride 3j (6.9 g, 100% yield), that was used in the next step without further purification.

### Step 9 (3S,3″S)-dimethyl 3,3″-((E)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylate) 3k; (S)-methyl 2-(1-ethyl-3-methyl-1H-pyrazole-5-carboxamido)-3-((E)-4-((S)-7-(methoxycarbonyl)-2-pivalamide-3,4-dihydro-5-oxa-1,2a-diazaacenaphthylen-3-yl)but-2-en-1-yl)-3,4-dihydro-5-oxa-1,2a-diazaacenaphthylene-7-carboxylate 3l; (3S,3″S)-dimethyl 3,3″-((E)-but-2-ene-1,4-diyl)bis(2-pivalamide-3,4-dihydro-5-oxa-1,2a-diazaacenaphthylene-7-carboxylate) 3m

To a solution of (3S,3′S)-dimethyl 3,3′-((E)-but-2-ene-1,4-diyl)bis(2-amino-3,4-dihydro-5-oxa-1,2a-diazaacenaphthylene-7-carboxylate) 3h (1.62 g, 3.12 mmol) and DIPEA (4.0 g, 31.2 mmol) in anhydrous tetrahydrofuran (20 mL) was added 1-ethyl-3-methyl-1H-pyrazole-5-carboxylic pivalic anhydride 3j (6.0 g, 25 mmol), at 0° C. After addition, the reaction was stirred from 0° C. for 20 minutes and warmed to ambient temperature and stirred for 16 hours. It was concentrated, and purified on a silica gel column, to the products. The most polar product was identified by ¹H NMR, ¹³C NMR and LCMS as (3S,3″S)-dimethyl 3,3″-((E)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylate) 3k (0.58 g, 24% yield). MS m/z (ESI): 791 [M+1]; ¹H NMR (400 MHz, CD₃OD): δ 7.69 (s, 2H), 7.51 (s, 2H), 6.73 (s, 2H), 5.67 (s, 2H), 4.67 (m, 6H), 4.42 (d, 11.2 Hz, 2H), 4.17 (d, 11.2 Hz, 2H), 3.97 (s, 6H), 2.65 (m, 4H), 2.31 (s, 6 H), 1.27 (m, 6H); ¹³C NMR (400 MHz, CD₃OD): (182.5; 166.4; 146.4; 141.5; 129.0; 127.3; 126.8; 120.2; 110.8; 110.2; 106.8; 68.8; 53.4; 52.5; 46.7; 38.4; 34.9; 27.1; 16.2; 13.2.

The second polar product was identified by ¹H NMR, ¹³C NMR and LCMS as (S)-methyl 2-(1-ethyl-3-methyl-1H-pyrazole-5-carboxamido)-3-((E)-4-((S)-7-(methoxycarbonyl)-2-pivalamido-3,4-dihydro-5-oxa-1,2a-diazaacenaphthylen-3-yl)but-2-en-1-yl)-3,4-dihydro-5-oxa-1,2a-diazaacenaphthylene-7-carboxylate 3l (1.01 g, 44% yield). MS m/z (ESI): 739[M+1]; ¹H NMR (400 MHz, CD₃OD: δ 7.85 (s, 1H), 7.73 (s, 1H), 7.58 (s, 1H), 7.52 (s, 1H), 7.28 (s, 1H), 6.67 (s, 1H), 6.45 (s, 1H), 5.64-5.46 (m, 6H), 4.90 (m, 2H), 4.73-4.44 (m, 10H), 3.98 (s, 3H), 2.30 (m, 2H), 1.54 (m, 3H), 1.25 (s, 9H).

The least polar product was identified by ¹H NMR, ¹³C NMR and LCMS as (3S,3″S)-dimethyl 3,3″-((E)-but-2-ene-1,4-diyl)bis(2-pivalamido-3,4-dihydro-5-oxa-1,2a-diazaacenaphthylene-7-carboxylate) 3m (0.56 g, 37% yield). MS m/z (ESI): 687[M+1]; ¹H NMR (400 MHz, Methanol-d₄): δ 7.76 (s, 2H), 7.50 (s, 2H), 5.72 (broad s, 2H), 5.54 (s, 2 H), 4.40 (d, 11.2 Hz, 2H), 4.23 (d, 11.2 Hz, 2H), 3.96 (s, 6H), 2.53 (m, 4H), 1.25 (s, 18H).

### Step 10 (3S,3″S)-3,3″-((E)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylic Acid) 3

To a solution of (3S,3″S)-dimethyl 3,3″-((E)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylate) 3k (570 mg, 0.72 mmol) in dioxane (8 mL) and water (2 mL) was added lithium hydroxide monohydrate (182 mg, 4.3 mmol), at 0° C. After addition, the reaction was stirred at ambient temperature for 16 hours. It was concentrated, and concentrated hydrochloric acid was added to bring its pH value to 4, and it was concentrated again. Then, ammonium hydroxide was added to change the pH to 9. The crude mixture was concentrated, and purified on reverse phase column to get (3S,3″S)-3,3″-((E)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylic acid) 3 (534 mg, 97.2% yield). MS m/z (ESI): 763[M+1]; ¹H NMR (400 MHz, CD₃OD): δ 7.72 (s, 2H), 7.61 (s, 2H), 6.71 (s, 2H), 5.55 (s, 2H), 4.60 (m, 6H), 4.32 (d, 11.2 Hz, 2H), 4.25 (d, 11.2 Hz, 2H), 2.65 (m, 4H), 2.31 (s, 6H), 1.17 (m, 6H).

### Example 4

### (S)-3-((E)-4-((S)-7-carbamoyl-2-(1-ethyl-3-methyl-1H-pyrazole-5-carboxamido)-3,4-dihydro-5-oxa-1,2a-diazaacenaphthylen-3-yl)but-2-en-1-yl)-2-pivalamido-3,4-dihydro-5-oxa-1,2a-diazaacenaphthylene-7-carboxamide 4

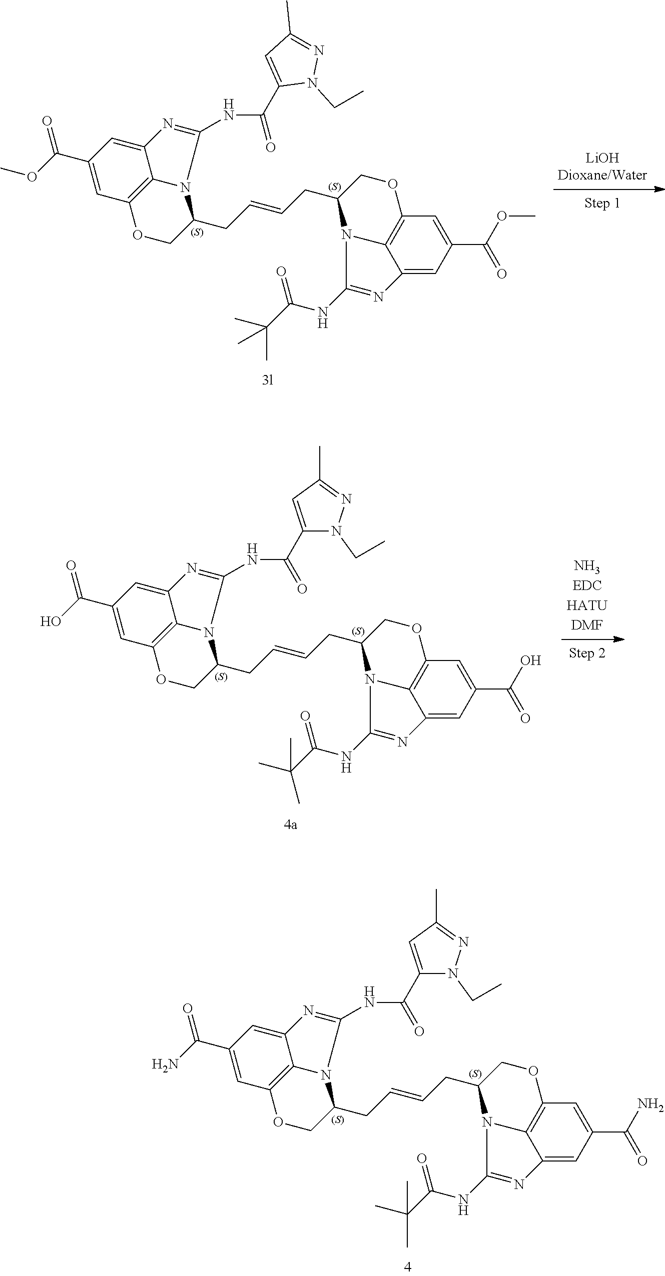

### Step 1 (S)-3-((E)-4-((S)-7-carboxy-2-(1-ethyl-3-methyl-1H-pyrazole-5-carboxamido)-3,4-dihydro-5-oxa-1,2a-diazaacenaphthylen-3-yl)but-2-en-1-yl)-2-pivalamido-3,4-dihydro-5-oxa-1,2a-diazaacenaphthylene-7-carboxylic Acid 4a

To a solution of (S)-methyl 2-(1-ethyl-3-methyl-1H-pyrazole-5-carboxamido)-3-((E)-4-((S)-7-(methoxycarbonyl)-2-pivalamido-3,4-dihydro-5-oxa-1,2a-diazaacenaphthylen-3-yl)but-2-en-1-yl)-3,4-dihydro-5-oxa-1,2a-diazaacenaphthylene-7-carboxylate 31 (0.85 g, 1.15 mmol) on dioxane (15 mL) and water (3 mL) was added lithium hydroxide monohydrate (145 mg, 43.45 mmol), at 0° C. After Addition, the reaction was stirred at ambient temperature for 16 hours. It was concentrated, and concentrated hydrochloric acid was added to bring its pH value to four, and it was concentrated again. Then, ammonium hydroxide was added to change the pH to 9. The crude mixture was concentrated, and purified on reverse phase column to get (S)-3-((E)-4-((S)-7-carboxy-2-(1-ethyl-3-methyl-1H-pyrazole-5-carboxamido)-3,4-dihydro-5-oxa-1,2a-diazaacenaphthylen-3-yl)but-2-en-1-yl)-2-pivalamido-3,4-dihydro-5-oxa-1,2a-diazaacenaphthylene-7-carboxylic acid 4a (801 mg, 97.8% yield). MS m/z (ESI): 711[M+1].

### Step 2 (S)-3-((E)-4-((S)-7-carbamoyl-2-(1-ethyl-3-methyl-1H-pyrazole-5-carboxamido)-3,4-dihydro-5-oxa-1,2a-diazaacenaphthylen-3-yl)but-2-en-1-yl)-2-pivalamido-3,4-dihydro-5-oxa-1,2a-diazaacenaphthylene-7-carboxamide 4

To a mixture of (S)-3-((E)-4-((S)-7-carboxy-2-(1-ethyl-3-methyl-1H-pyrazole-5-carboxamido)-3,4-dihydro-5-oxa-1,2a-diazaacenaphthylen-3-yl)but-2-en-1-yl)-2-pivalamido-3,4-dihydro-5-oxa-1,2a-diazaacenaphthylene-7-carboxylic acid in N,N-dimethylformamide (10 mL), was added HATU (1.75 g, 4.6 mmol) and EDCI (0.88 g, 4.6 mmol). After addition, it was stirred at ambient temperature for 30 minute, and ammonia gas was bubbled in for 1 minute. Then, it was absorbed onto silica gel, and eluted with 20% methanol (with 7 N ammonia) in dichloromethane, to get the title compound, (S)-3-((E)-4-((S)-7-carbamoyl-2-(1-ethyl-3-methyl-1H-pyrazole-5-carboxamido)-3,4-dihydro-5-oxa-1,2a-diazaacenaphthylen-3-yl)but-2-en-1-yl)-2-pivalamido-3,4-dihydro-5-oxa-1,2a-diazaacenaphthylene-7-carboxamide 4 (693 mg, 85% yield over 2 steps). MS m/z (ESI): 709[M+1]; ¹H NMR (400 MHz, CD₃OD): δ 7.77 (s, 1H), 7.64 (d, 1H), 7.40 (s, 1H), 7.35 (d, 1H), 6.64 (s, 1H), 5.60-5.50 (m, 2H), 5.01-4.90 (m, 7H), 4.71 (m, 2H), 4.60 (m, 1H), 4.50 (m, 2H), 4.26 (m, 2H), 2.66 (m, 1H), 2.57 (m, 1H), 2.46 (m, 2H), 2.24 (s, 3H), 1.39 (t, 3H), 1.28 (s, 9H).

### Example 1 (Method B)

### (3S,3″S)-3,3″-((E)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxamide) 1

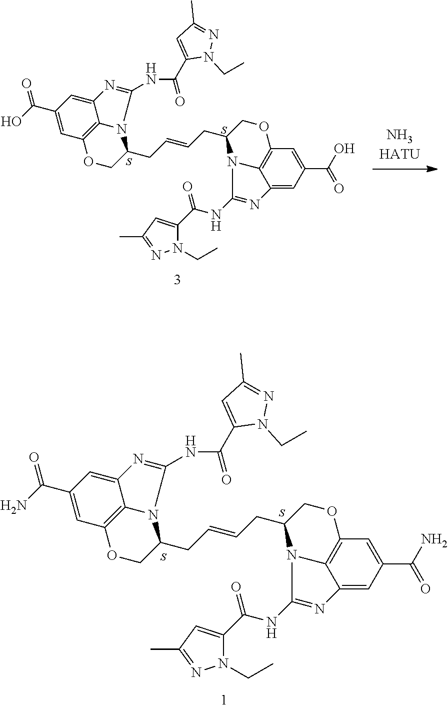

The title compound can be prepared by the same method as step 2 in example 4.

### Example 2 (Method C)

### (3S,3″S)-3,3″-((Z)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxamide) 2

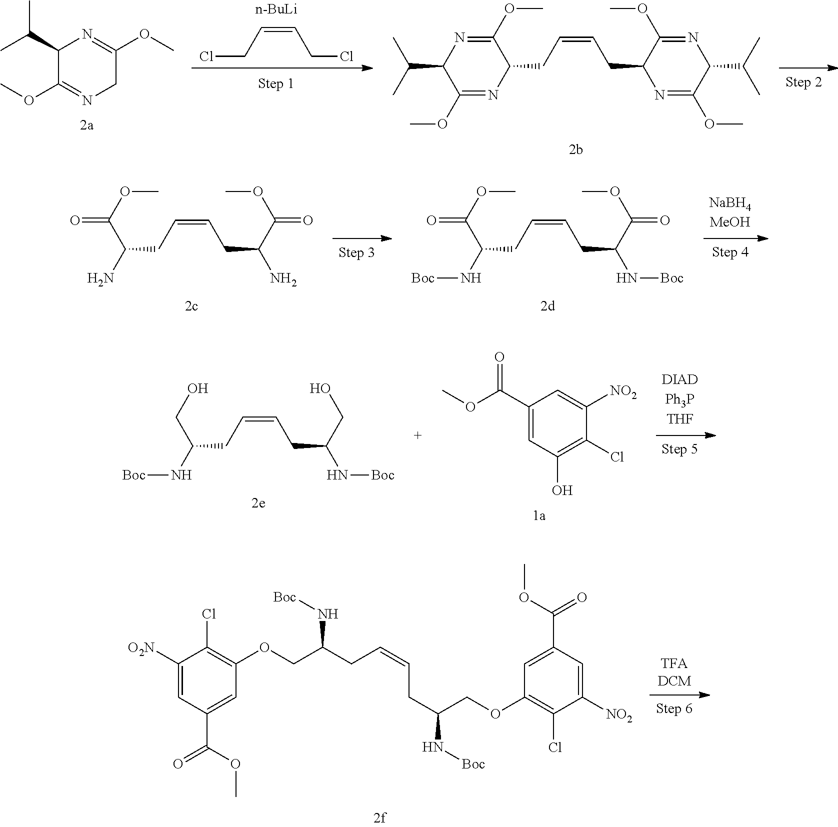

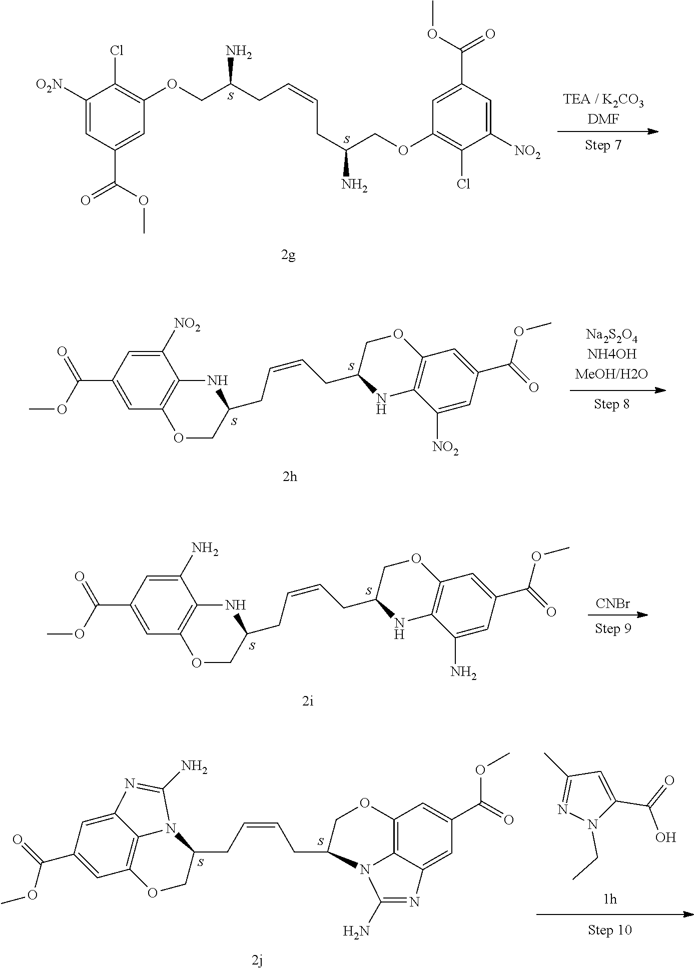

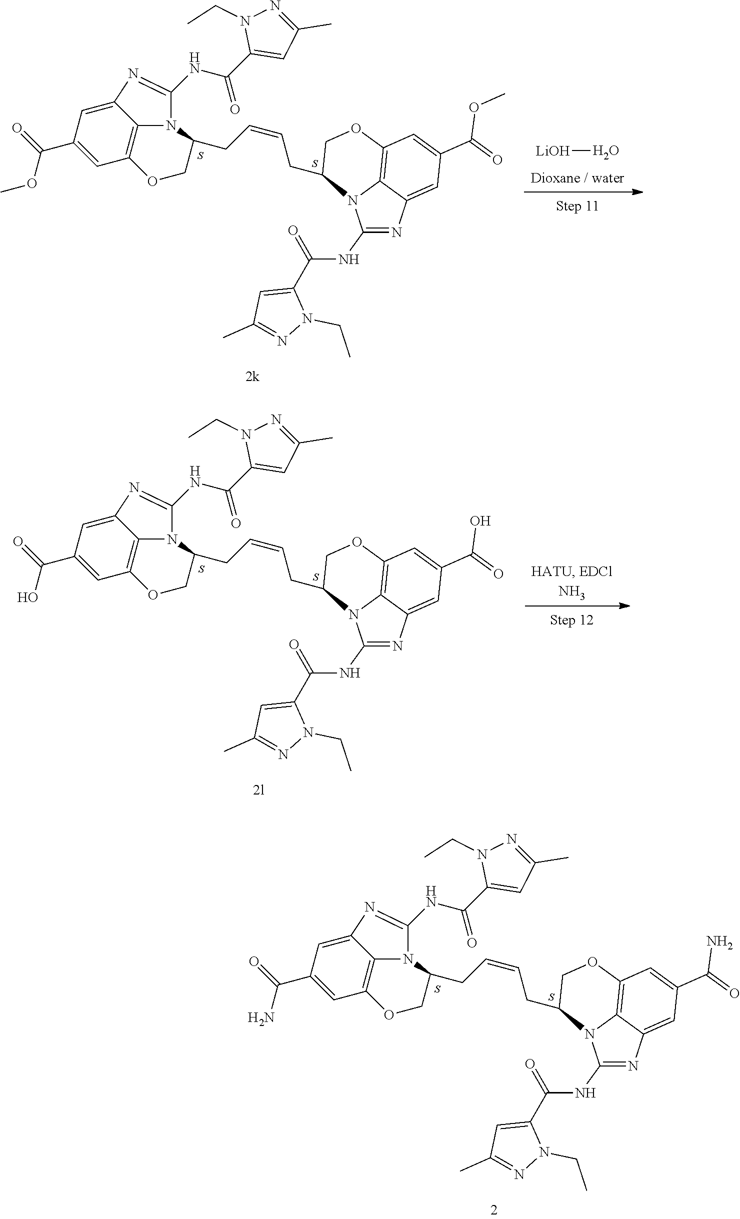

### Method C

### Step 1 (Z)-1,4-bis((2S,5R)-5-isopropyl-3,6-dimethoxy-2,5-dihydropyrazin-2-yl)but-2-ene 2b

To a solution of (R)-2-isopropyl-3,6-dimethoxy-2,5-dihydropyrazine 2a (26.5 g, 144 mmol) in THF (300 mL), at −78° C., was added n-Butyllithium (1.6 M in THF, 252 mL, 403 mmol). The reaction was stirred at −78° C. for 30 minutes and a solution of cis-1,4-dichloro-2-butene (6.0 g, 48 mmoL) in THF (20 mL) was added dropwise. After addition, the reaction was slowly warmed to ambient temperature, and stirred for 10 hours. The reaction was worked up with sat. NaHCO₃, and extracted with ether. The organic phase was concentrated and purified on a silica gel column, eluting with 40% ethyl acetate in dichloromethane, to get the desired product (Z)-1,4-bis((2S,5R)-5-isopropyl-3,6-dimethoxy-2,5-dihydropyrazin-2-yl)but-2-ene 2b (21.2 g, 97.5% yield). MS m/z (ESI): 421 [M+1]; ¹H NMR (400 MHz, CDCl₃): δ 5.40 (t, 4.68 Hz, 2H), 4.11 (m, 2H), 3.93 (t, 3.36 Hz, 2H), 3.70 (s, 6H), 3.68 (s, 6H), 2.58 (m, 4H), 2.27 (m, 2H), 1.05 (d, 6.88 Hz, 6H), 0.69 (d, 6.88 Hz, 6H). C₁₃NMR (CDCl₃, 400 mHz): 164.3520, 155.5372, 155.2789, 149.3704, 133.4904, 129.8941, 121.0590, 118.8352, 117.8400, 116.0490, 79.9376, 70.9526, 53.0272, 49.1954, 35.9561, 28.3666

### Step 2 (2S,7S,Z)-dimethyl 2,7-diaminooct-4-enedioate 2c

To a solution of (Z)-1,4-bis((2S,5R)-5-isopropyl-3,6-dimethoxy-2,5-dihydropyrazin-2-yl)but-2-ene 2b (10 g, 19.21 mmol) in 1,4-dioxane (200 mL), at ambient temperature, was added 0.3 M hydrochloric acid (200 mL). The reaction was stirred at ambient temperature for 16 hours. TLC showed that the start material was completely converted. It was flushed with compressed air for 20 minutes and concentrated at room temperature to bring its pH value to about 7. Then, 7 N ammonia solution in methanol was added to bring its pH value to about 9. It was concentrated again to get rid of the excess methanol. The residue was Lyophilized to get (2S,7S, Z)-dimethyl 2,7-diaminooct-4-enedioate 2c (4.2 g, 95% yield). MS m/z (ESI): 231[M+1]; ¹H NMR (400 MHz, CDCl₃): δ 5.56 (m, 2H), 3.72 (s, 6H), 3.56 (dd, 5.20 Hz, 7.28 Hz, 2H), 2.58-2.39 (m, 2H), 1.64 (broad s, 4H); ¹³CNMR (400 MHz, CDCl₃):175.7336, 128.0116, 54.1739, 52.1016, 32.5440.

### Step 3 (2S,7S,Z)-dimethyl 2,7-bis((tert-butoxycarbonyl)amino)oct-4-enedioate 2d

To a solution of (2S,7S,Z)-dimethyl 2,7-diaminooct-4-enedioate 2c (6.9 g, 30 mmol) in dichloromethane (300 mL), at ambient temperature, was added DMAP (3.7 g, 30 mmol). It was cooled to 0° C., and Boc anhydride (19.7 g, 90 mmol) was added. The reaction was stirred at 0° C. overnight, and then, slowly warmed to ambient temperature. Hunig's base (3.9 g, 30 mmol) was added and it was stirred for 1 hour. It was concentrated and purified on a silica gel column, eluting with 50% ethyl acetate in hexanes, to get the desired product, (2S,7S, Z)-dimethyl 2,7-bis((tert-butoxycarbonyl)amino)oct-4-enedioate 2d (7.8 g, 60% yield). MS m/z (ESI): 431[M+1]; ¹H NMR (400 MHz, CDCl₃): δ 5.49 (dd, 5.2 Hz, 4.8 Hz, 2H), 5.19 (d, 7.8 Hz, 2H), 4.43 (m, 2H), 3.76 (s, 6H), 2.62-2.57 (m, 2H), 2.49-2.44 (m, 2H), 1.50 (s, 18H); ¹³CNMR (400 MHz, CDCl₃): 172.4101, 155.1458, 127.3543, 80.1546, 52.8811, 52.4108, 30.4195, 28.2974.

### Step 4 di-tert-butyl ((2S,7S,Z)-1,8-dihydroxyoct-4-ene-2,7-diyl)dicarbamate 2e

To a solution of (2S,7S,Z)-dimethyl 2,7-bis((tert-butoxycarbonyl)amino)oct-4-enedioate 2d (5.6 g, 13 mmol) in anhydrous methanol (100 mL) was added sodium borohydride (2.0 g, 54 mmol) portion wise at 0° C. After addition, the reaction was warmed up and stirred at ambient temperature for 16 hours. Acetic acid was added to adjust the pH value to about 5 and it was concentrated. The residue was dissolved in dichloromethane (100 mL). It was filtered. The filtrate was concentrated and purified on a silica gel column, eluting with 60% ethyl acetate in hexanes to get di-tert-butyl ((2S,7S, Z)-1,8-dihydroxyoct-4-ene-2,7-diyl)dicarbamate 2e (3.1 g, 63.7% yield). MS m/z (ESI): 375[M+1]; ¹H NMR (400 MHz, CDCl₃): δ 5.51 (m, 2H), 5.32 (broad s, 2H), 3.65 (m, 6H), 2.37-2.42 (m, 4H), 1.46 (s, 18H); ¹³C NMR (400 MHz, CDCl₃): 156.1859; 156.0863, 128.1596, 128.1019, 79.7261, 63.8966, 52.1977, 29.0973, 28.4069.

### Step 5 dimethyl 5,5′-(((2S,7S,Z)-2,7-bis((tert-butoxycarbonyl)amino)oct-4-ene-1,8-diyl)bis(oxy))bis(4-chloro-3-nitrobenzoate) 2f

A solution of diisopropyl azodicarboxylate (9.73 g, 48.1 mmol) and triphenyl phosphene in THF (100 mL) was stirred at 0° C. for 10 minutes to form a white waxy precipitate. Then, methyl 4-chloro-3-hydroxy-5-nitrobenzoate 1a (11.14 g, 48.1 mmoL) was added, followed by the di-tert-butyl ((2S,7S, Z)-1,8-dihydroxyoct-4-ene-2,7-diyl)dicarbamate 2e (6.0 g, 16.02 mmol). The reaction was stirred at 0° C. for 10 hours, and slowly warmed to ambient temperature, and stirred for 2 hours. It was concentrated. The crude stuff was purified on a silica gel column, eluting with 60% ethyl acetate in hexanes, to get dimethyl 5,5′-(((2S,7S,Z)-2,7-bis((tert-butoxycarbonyl)amino)oct-4-ene-1,8-diyl)bis(oxy))bis(4-chloro-3-nitrobenzoate) 2f (8.1 g, 21% yield). MS m/z (ESI): 823[M+Na].

### Step 6 dimethyl 5,5′-(((2S,7S,Z)-2,7-diaminooct-4-ene-1,8-diyl)bis(oxy))bis(4-chloro-3-nitrobenzoate) 2g

To a solution of dimethyl 5,5′-(((2S,7S,Z)-2,7-bis((tert-butoxycarbonyl)amino)oct-4-ene-1,8-diyl)bis(oxy))bis(4-chloro-3-nitrobenzoate) 2f (8.1 g, 10.1 mmol) in dichloromethane (200 mL) was added trifluoro acetic acid (40 mL). After addition, the reaction was stirred at ambient temperature for 12 hours. LCMS showed that the reaction was done. It was concentrated. The crude residue was dissolved in MeOH (100 mL), and sodium bicarbonate was added to make it basic (pH 8). It was concentrated again and absorbed onto silica gel and was purified on a silica gel column, eluting with 60% ethyl acetate in hexanes, to get dimethyl 5,5′-(((2S,7S,Z)-2,7-diaminooct-4-ene-1,8-diyl)bis(oxy))bis(4-chloro-3-nitrobenzoate) 2g (5.83 g, 96% yield). ¹H NMR (400 mHz, CD₃OD): δ 8.11 (d, 1.72 Hz, 2H), 7.88 (d, 1.72 Hz, 2H), 5.82 (t, 4.96 Hz, 2H), 4.47-4.33 (m, 4H), 3.98 (s, 6H), 3.73 (m, 2H), 2.88-2.66 (m, 4 H).

### Step 7 (3S,3′S)-dimethyl 3,3′-((Z)-but-2-ene-1,4-diyl)bis(5-nitro-3,4-dihydro-2H-benzo[b][1,4]oxazine-7-carboxylate) 2h

To a solution of dimethyl 5,5′-(((2S,7S,Z)-2,7-diaminooct-4-ene-1,8-diyl)bis(oxy))bis(4-chloro-3-nitrobenzoate) 2g (5.8 g, 9.62 mmol) in DMF (60 mL) was added triethylamine (6.1 g, 60 mmol) and potassium carbonate (12.5 g, 90 mmol). After addition, the reaction was stirred at 100° C. for 2 hours. LCMS showed that the reaction was done. It was concentrated and absorbed onto silica gel. It was eluted with 20% ethyl acetate in dichloromethane to get the titled compound, (3S,3′S)-dimethyl 3,3′-((Z)-but-2-ene-1,4-diyl)bis(5-nitro-3,4-dihydro-2H-benzo[b][1,4]oxazine-7-carboxylate) 2h (3.00 g, 59% yield). MS m/z (ESI): 529 [M+1]; ¹H NMR (400 MHz, DMSO-d₆): δ 8.77 (d, 3.32 Hz, 2H), 8.22 (d, 3.32 Hz, 2H), 5.67 (m, 2H), 4.07 (m, 4H), 3.78 (s, 6H), 3.70 (m, 2H), 2.32 (m, 4H).

### Step 8 (3S,3′S)-dimethyl 3,3′-((Z)-but-2-ene-1,4-diyl)bis(5-amino-3,4-dihydro-2H-benzo[b][1,4]oxazine-7-carboxylate) 2i

To a solution of (3S,3′S)-dimethyl 3,3′-((Z)-but-2-ene-1,4-diyl)bis(5-nitro-3,4-dihydro-2H-benzo[b][1,4]oxazine-7-carboxylate) 2g (2.7 g, 4.5 mmol) in anhydrous methanol (40 mL) was added Na₂S₂O₄ (19.5 g, 112 mmoL) in water (40 mL), followed by concentrated ammonium hydroxide solution (40 mL). After addition, the reaction was stirred at ambient temperature for 4 hours. LCMS showed that the reaction was done. It was extracted with ethyl acetate. The organic layer was concentrated and purified on a silica gel column, eluted with 20% ethyl acetate in DCM, to get the titled compound, (3S,3′S)-dimethyl 3,3′-((Z)-but-2-ene-1,4-diyl)bis(5-amino-3,4-dihydro-2H-benzo[b][1,4]oxazine-7-carboxylate) 2i (2.0 g, 69%). MS m/z (ESI): 469[M+1].

### Step 9 (3S,3′S)-dimethyl 3,3′-((Z)-but-2-ene-1,4-diyl)bis(2-amino-3,4-dihydro-5-oxa-1,2a-diazaacenaphthylene-7-carboxylate) 2j

To a suspension of (3S,3′S)-dimethyl 3,3′-((Z)-but-2-ene-1,4-diyl)bis(5-amino-3,4-dihydro-2H-benzo[b][1,4]oxazine-7-carboxylate) 2i (2.0 g, 4.27 mmol) in DMF (100 mL) was added cyanogen bromide (4.8 g, 45 mmol). After addition, the reaction was stirred at ambient temperature for 16 hours to get a clear solution. LCMS showed that the start material was completely converted. It was concentrated. The crude residue was purified on a silica gel column, eluting with 20% methanol (containing 7 N ammonia) in dichloromethane, to get (3S,3′S)-dimethyl 3,3′-((Z)-but-2-ene-1,4-diyl)bis(2-amino-3,4-dihydro-5-oxa-1,2a-diazaacenaphthylene-7-carboxylate) 2j (2.0 g, 90% yield); MS m/z (ESI): 519[M+1].

### Step 10 (3S,3′S)-dimethyl 3,3′-((Z)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylate) 2k

To a solution of 1-ethyl-3-methyl-1H-pyrazole-5-carboxylic acid 1h (2.4 g, 15.4 mmoL) in DMF (50 mL) was added EDCI (3.7 g, 19.3 mmoL) and HATU (7.3 g, 19.3 mmoL) and DMAP (1.9 g, 15.4 mmoL). The reaction was stirred at ambient temperature for 20 minutes to see that the acid was converted to the HATU complex (MW=272). Then, the (3S,3′S)-dimethyl 3,3′-((Z)-but-2-ene-1,4-diyl)bis(2-amino-3,4-dihydro-5-oxa-1,2a-diazaacenaphthylene-7-carboxylate) 2j (2.0 g, 3.85 mmol) was added. The reaction was stirred at room temperature for 2 hours. LCMS showed that the reaction was done. It was absorbed onto silica gel and purified on a silica gel column, eluting with 100% ethyl acetate in dichloromethane to get (3S,3′S)-dimethyl 3,3′-((Z)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylate) 2k (1.3 g, 43% yield). MS m/z (ESI):791[M+1]

### Step 11 (3S,3′S)-3,3′-((Z)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylic Acid) 2l

To a (3S,3′S)-dimethyl 3,3′-((Z)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylate) 2k (1.2 g, 1.52 mmol) in 1,4-dioxane (10 mL) and water (2 mL) was added lithium hydroxide hydrate (320 mg, 7.6 mmol), at 0° C. After addition, the reaction was stirred at ambient temperature for 16 hours. It was concentrated, and concentrated hydrochloric acid was added to bring the pH value to about 4. Then, ammonium hydroxide was added to bring the pH value to 9. The crude mixture was concentrated, and the crude (3S,3′S)-3,3′-((Z)-but-2-ene-1,4-diyl) bis(2-(1-ethyl-3-methyl-1H-pyrazole-5-carboxamido)-3,4-dihydro-5-oxa-1,2a-diazaacenaphthylene-7-carboxylic acid) 2l was used in the next step, without further purification. A small amount was purified on reverse phase column. MS m/z (ESI): 763[M+1].

### Step 12 (3S,3″S)-3,3″-((Z)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxamide) 2

To a solution of (3S,3′S)-3,3′-((Z)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxylic acid) 21 (directly from the previous step) in DMF (20 mL) was added EDCI (1.02 g, 5.32 mmol) and HATU (2.01 g, 5.32 mmoL). After addition, the reaction was stirred at ambient temperature for 1 hour to see the formation of the HATU complex. At this point, ammonia gas was bubbled in for 1 minutes to see the complete conversion by LCMS. It was concentrated, and absorbed onto silica gel and was eluted with 15% methanol (containing 7 N ammonia) in dichloromethane to get (3S,3″S)-3,3″-((Z)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxamide) 2 (470 mg, 40.5% yield over 2 steps).

### Example 5

### (3S,3″S)-3,3″-(butane-1,4-diyl)bis(2-(1-ethyl-3-methyl-1H-pyrazole-5-carboxamido)-3,4-dihydro-5-oxa-1,2a-diazaacenaphthylene-7-carboxamide) 5

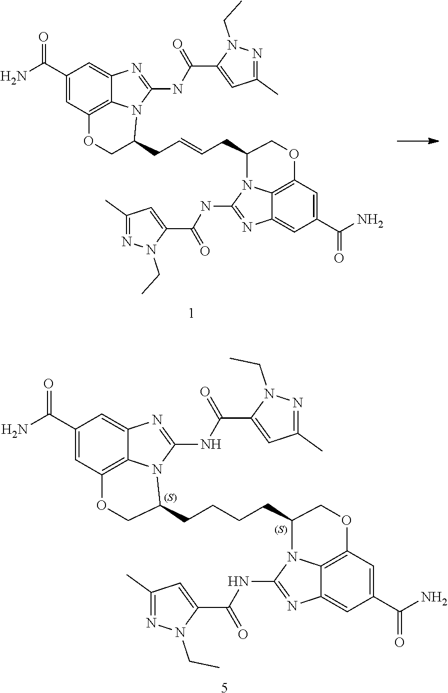

To a solution of compound (3S,3″S)-3,3″-((E)-but-2-ene-1,4-diyl)bis(2-(1-ethyl-3-methyl-1H-pyrazole-5-carboxamido)-3,4-dihydro-5-oxa-1,2a-diazaacenaphthylene-7-carboxamide) 1 (7 mg, 0.0092 mmol) in MeOH (0.4 mL)/THF (0.4 mL) was charged with 10 wt % Pd/C (8 mg). The mixture was stirred under H₂ balloon for 4 h. The crude product was purified by silica gel column chromatography with elution system of MeOH/DCM to give title compound 5 (3S,3″S)-3,3″-(butane-1,4-diyl)bis(2-(1-ethyl-3-methyl-1H-pyrazole-5-carboxamido)-3,4-dihydro-5-oxa-1,2a-diazaacenaphthylene-7-carboxamide) (3 mg). MS m/z (ESI): 763 [M+1]; ¹H NMR (500 MHz, Methanol-d₄) δ 7.45-7.40 (m, 2H), 7.18 (d, J=1.1 Hz, 2H), 6.35 (s, 2H), 4.65 (dd, J=13.6, 7.0 Hz, 2H), 4.52 (t, J=9.2 Hz, 4H), 4.43 (dq, J=14.1, 7.2 Hz, 2H), 4.09 (dd, J=11.9, 2.6 Hz, 2H), 2.12-1.97 (m, 2H), 1.81 (s, 8H), 1.67-1.47 (m, 4H), 1.28 (t, J=7.1 Hz, 6H).

### Example 6

### (3S,3″S)-3,3″-(but-2-yne-1,4-diyl)bis(2-(1-ethyl-3-methyl-1H-pyrazole-5-carboxamido)-3,4-dihydro-5-oxa-1,2a-diazaacenaphthylene-7-carboxamide) 6

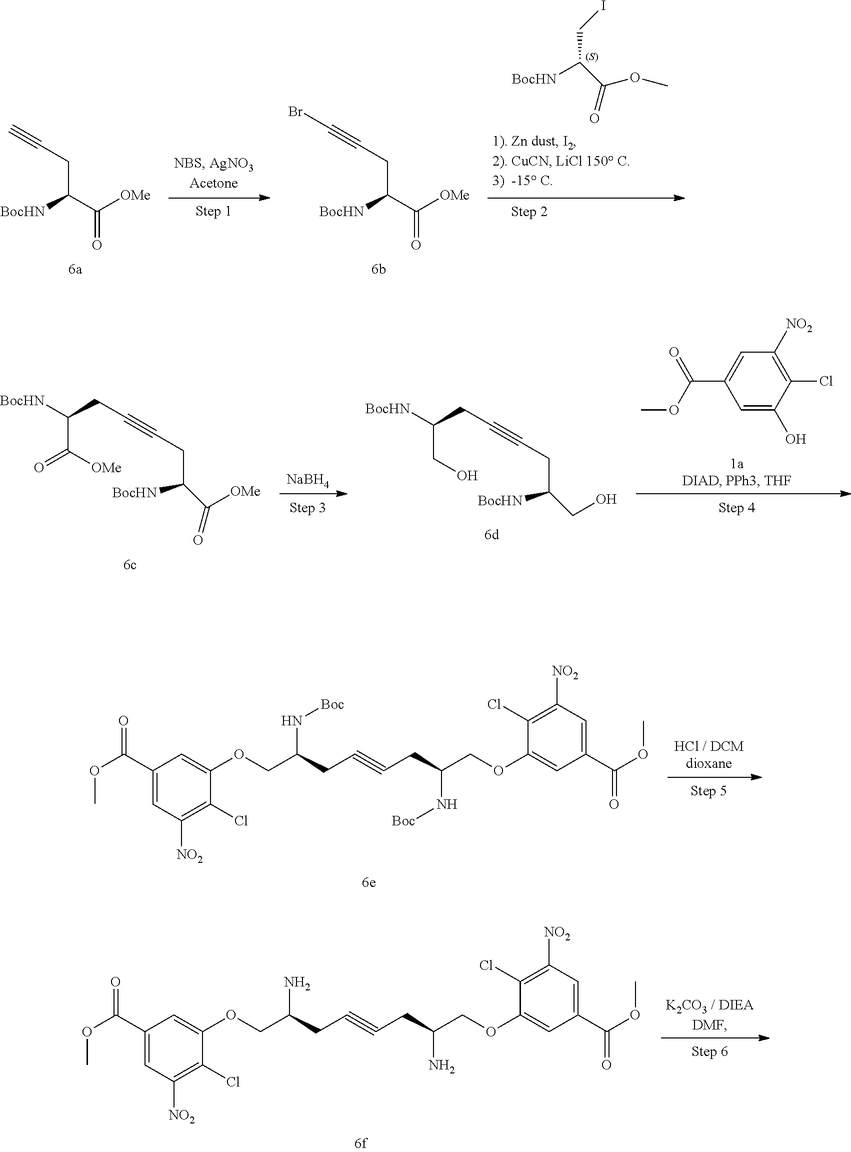

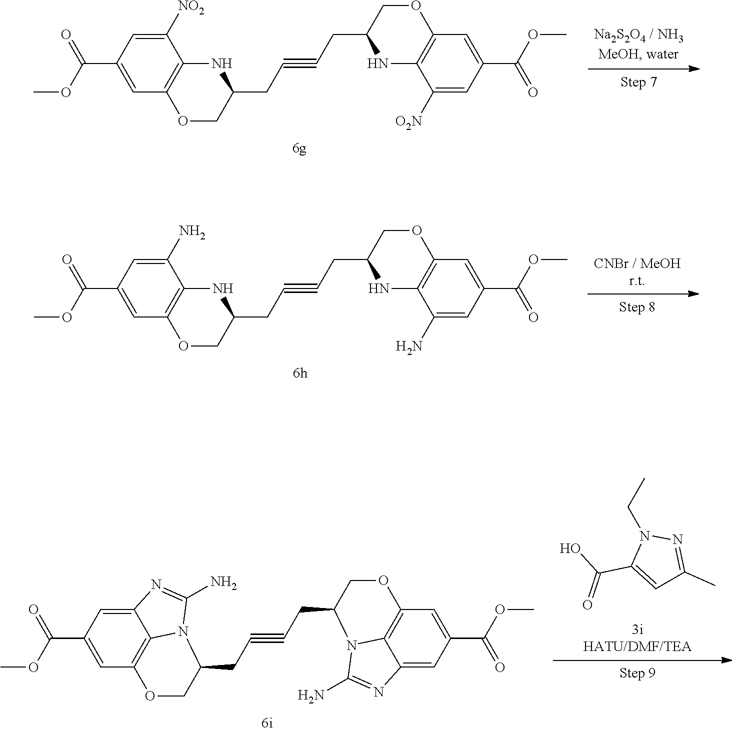

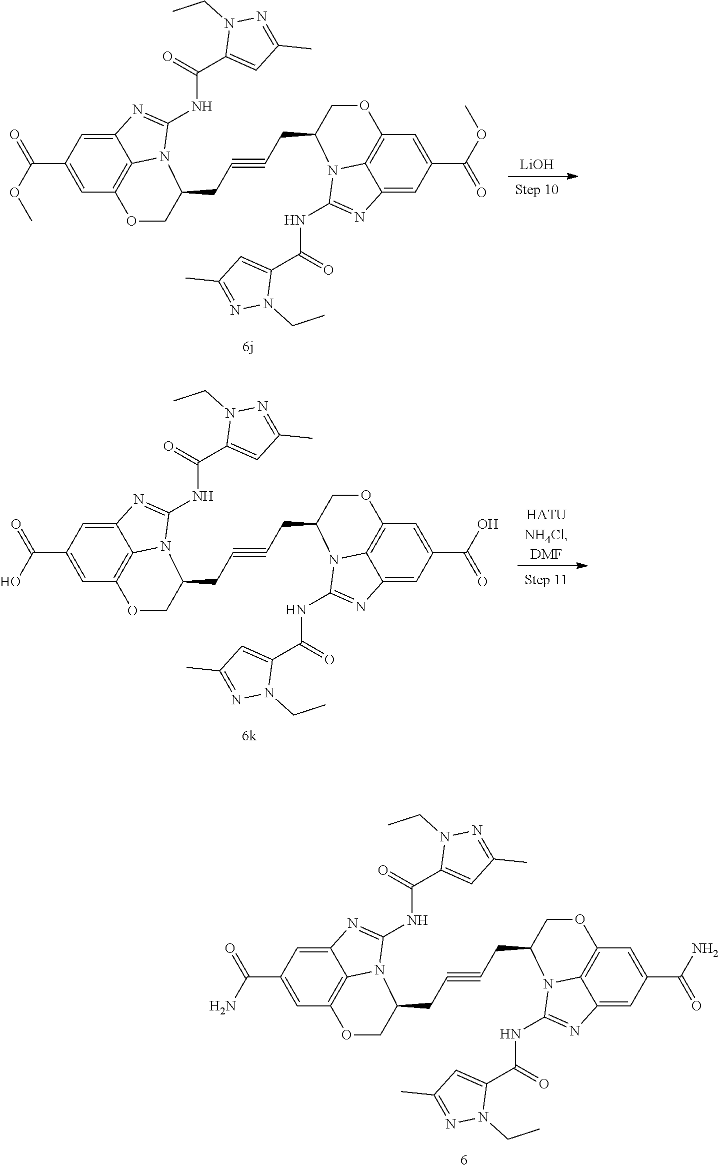

### Step 1 (S)-methyl 5-bromo-2-((tert-butoxycarbonyl)amino)pent-4-ynoate 6b

N-Bromosuccinimide (1.2 g, 7 mmol) and AgNO₃ (100 mg, 0.60 mmol) were added to a stirred solution of (S)-methyl 2-((tert-butoxycarbonyl)amino)pent-4-ynoate 6a (2.2 g, 4 mmol) in acetone (15 mL) under argon. The reaction mixture was stirred for 7 hours at room temperature. After this time, water (50 mL) was added and the suspension was extracted with ethyl acetate (3×100 mL). The combined organic layers were then washed with water (50 mL) and brine (50 mL), dried over anhydrous sodium sulphate and concentrated in vacuo. The crude product was purified via flash column chromatography (9:1 hexane:ethyl acetate) to yield (S)-methyl 5-bromo-2-((tert-butoxycarbonyl)amino)pent-4-ynoate 6b (670 mg).

### Step 2 (2S,7S)-dimethyl 2,7-bis((tert-butoxycarbonyl)amino)oct-4-ynedioate 6c

Zinc dust (662 mg, 10.1 mmol) was weighed into a round bottomed flask. Iodine (24 mg, 0.1 mmol) was added and the flask was heated with a heat gun, under vacuum for ten minutes and then flushed with argon. The flask was vacuumed and flushed with argon a further three times and cooled to 0° C. (S)-Methyl 2-(tert-butoxycarbonylamino)-3-iodopropanoate (1.1g, 3 mmol, purchased from Combi-Blocks) was dissolved in anhydrous DMF (1.5 mL) and added dropwise via syringe to the activated zinc at 0° C. The reaction mixture was then allowed to warm to room temperature and stirred for 90 minutes to give the corresponding organozinc intermediate (TLC analysis was used to confirm the complete consumption of the starting material). In a separate flask, CuCN (236 mg, 2.6 mmol) and LiCl (224 mg, 5.2 mmol) were heated to 150° C. for two hours under argon and then cooled to room temperature. DMF (4 mL) was added and the solution stirred for five minutes to form the soluble CuCN-2LiCl complex. The copper complex was then cooled to −15° C. Once the zinc insertion process was judged to have reached completion, stirring was ceased to allow the zinc powder to settle to the bottom of the flask. The supernatant was removed via syringe under argon (with care being taken to avoid the transfer of zinc) and added dropwise to the copper complex at −15° C. (S)-methyl 5-bromo-2-((tert-butoxycarbonyl)amino)pent-4-ynoate 6b (0.67g, 2.01 mmol) was then dissolved in DMF (1.5 mL) and added dropwise to the copper complex at −15° C. The cooling bath was removed, and the reaction mixture was stirred at room temperature for 16 hours under argon. After this time, water (50 mL) was added and the suspension was extracted with diethyl ether (3×100 mL), washed with brine (60 mL), dried over anhydrous sodium sulphate and concentrated in vacuo. The crude product was purified via flash column chromatography (5:1 hexane:ethyl acetate) to yield (2S,7S)-dimethyl 2,7-bis((tert-butoxycarbonyl)amino)oct-4-ynedioate 6c (670 mg, 53%).

### Step 3 di-tert-butyl ((2S,7S)-1,8-dihydroxyoct-4-yne-2,7-diyl)dicarbamate 6d

To the THF (15 mL) solution of (2S,7S)-dimethyl 2,7-bis((tert-butoxycarbonyl)amino)oct-4-ynedioate 6c (670 mg, 2.55 mmol) at 0° C. was added NaBH₄ (290 mg, 7.66 mmol, 3 eq) in 2 mL MeOH, The mixture was stirred at 0° C. for 30 min. 1M HCl was added to adjust pH value to ˜5 and concentrated. The residue was dissolved in DCM, filtered and concentrated under vacuum to give crude compound, which was purified by column (hexane:EA=40%:60%) to get desired product di-tert-butyl ((2S,7S)-1,8-dihydroxyoct-4-yne-2,7-diyl)dicarbamate 6d (540 mg).

### Step 4 dimethyl 5,5′-(((2S,7S)-2,7-bis((tert-butoxycarbonyl)amino)oct-4-yne-1,8-diyl)bis(oxy))bis(4-chloro-3-nitrobenzoate) 6e

To the THF solution (˜10 mL) was added PPh₃ (428 mg, 3 eq) followed with DEAD (257 uL, 3 eq), the reaction mixture was stirred at 0° C. temperature for 10 mins. Then add Methyl 4-chloro-3-hydroxy-5-nitrobenzoate (379 mg, 3 eq) in 2 ml THF into reaction and followed by di-tert-butyl ((2S,7S)-1,8-dihydroxyoct-4-yne-2,7-diyl)dicarbamate 6d (540 mg). The reaction was stirred at room temperature for overnight. The mixture was remove solvent under vacuum and purified by silica gel column (24g ISCO cartridge with 40% EtOAc in Hexane) to give title compound dimethyl 5,5′-(((2S,7S)-2,7-bis((tert-butoxycarbonyl)amino)oct-4-yne-1,8-diyl)bis(oxy))bis(4-chloro-3-nitrobenzoate) 6e (498 mg, 52%).

### Step 5 dimethyl 5,5′-(((2S,7S)-2,7-diaminooct-4-yne-1,8-diyl)bis(oxy))bis(4-chloro-3-nitrobenzoate) 6f

To the methylene chloride solution (˜10 mL) of dimethyl 5,5′-(((2S,7S)-2,7-diaminooct-4-yne-1,8-diyl)bis(oxy))bis(4-chloro-3-nitrobenzoate) 6e (180 mg) was added 4N HCl in dioxane (3 mL, 15 mmol), the reaction mixture was stirred at room temperature for 3 hours. The volatile was evaporated under vacuum to give title compound dimethyl 5,5′-(((2S,7S)-2,7-diaminooct-4-yne-1,8-diyl)bis(oxy))bis(4-chloro-3-nitrobenzoate) 6f (125 mg, 96%). MS m/z (ESI): 600 [M+1]

### Step 6 (3S,3′S)-dimethyl 3,3′-(but-2-yne-1,4-diyl)bis(5-nitro-3,4-dihydro-2H-benzo[b][1,4]oxazine-7-carboxylate) 6g

To the DMF solution (˜4 mL) of dimethyl 5,5′-(((2S,7S)-2,7-diaminooct-4-yne-1,8-diyl)bis(oxy))bis(4-chloro-3-nitrobenzoate) 6f (125 mg) was added triethylamine (150 uL, 1.0 mmol) followed with K₂CO₃ (280 mg, 2.0 mmol), the reaction mixture was heated at 100° C. for 3 hours. The mixture was cooled down to room temperature, water (50 mL) was added and the suspension was extracted with ethyl acetate (3×100 mL). The combined organic layers were then washed with water (50 mL) and brine (50 mL), dried over anhydrous sodium sulphate and concentrated in vacuo. The crude product was purified via flash column chromatography (9:1 DCM: Methanol) to yield (3S,3′S)-dimethyl 3,3′-(but-2-yne-1,4-diyl)bis(5-nitro-3,4-dihydro-2H-benzo[b][1,4]oxazine-7-carboxylate) 6g (80 mg). MS m/z (ESI): 527 [M+1]

### Step 7 (3S,3′S)-dimethyl 3,3′-(but-2-yne-1,4-diyl)bis(5-amino-3,4-dihydro-2H-benzo[b][1,4]oxazine-7-carboxylate) 6h

To the MeOH solution (˜15 mL) of (3S,3′S)-dimethyl 3,3′-(but-2-yne-1,4-diyl)bis(5-nitro-3,4-dihydro-2H-benzo[b][1,4]oxazine-7-carboxylate) 6g was added Na₂S₂O₄ (630 mg, 3.62 mmol, 10 eq) in water (5 mL), followed with conc. NH₄OH (0.78 mL, 10.8 mmol), the reaction mixture was stirred at room temperature for 1 hour. The mixture was diluted with water (20 mL), extracted with EtOAc (30 mL×3). Organic layers were combined, washed with brine (20 mL×1), dried over Na₂SO₄, filtered and the filtrated was concentrated under vacuum to give crude product, which was purified by silica gel column (12g cartridge with 10% methanol in DCM) to give title compound (3S,3′S)-dimethyl 3,3′-(but-2-yne-1,4-diyl)bis(5-amino-3,4-dihydro-2H-benzo[b][1,4]oxazine-7-carboxylate) 6h (60 mg). MS m/z (ESI): 467 [M+1]

### Step 8 (3S,3′S)-dimethyl 3,3′-(but-2-yne-1,4-diyl)bis(2-amino-3,4-dihydro-5-oxa-1,2a-diazaacenaphthylene-7-carboxylate) 6i

To the MeOH solution (˜20 mL) of (3S,3′S)-dimethyl 3,3′-(but-2-yne-1,4-diyl)bis(5-amino-3,4-dihydro-2H-benzo[b][1,4]oxazine-7-carboxylate) 6h (62 mg) was added BrCN (140 mg, 1.5 mmol), the reaction mixture was stirred at room temperature for overnight. The mixture was concentrated under vacuum to give crude dimethyl (3S,3′S)-dimethyl 3,3′-(but-2-yne-1,4-diyl)bis(2-amino-3,4-dihydro-5-oxa-1,2a-diazaacenaphthylene-7-carboxylate) 6i (65 mg, 80%), which was used in the next step without further purification. MS m/z (ESI): 517 [M+1]

### Step 9 (3S,3′S)-dimethyl 3,3′-(but-2-yne-1,4-diyl)bis(2-(1-ethyl-3-methyl-1H-pyrazole-5-carboxamido)-3,4-dihydro-5-oxa-1,2a-diazaacenaphthylene-7-carboxylate) 6j

To the DCM(˜15 mL) and DMF (˜3 mL) solution of 6i (65 mg) was added 1-ethyl-3-methyl-1H-pyrazole-5-carboxylic acid 3i (44 mg, 0.28 mmol), HATU (136 mg, 3 eq) and TEA (155 uL, 5 eq) the reaction mixture was stirred at room temperature for overnight. The Mixture was diluted with DCM (30 mL), washed with water (10 mL), dried over Na₂SO₄, filtered and the filtrated was concentrated under vacuum. The residue was purified by prep. HPLC (10-100% water: ACN (1% TFA)) to give dimethyl (3S,3′S)-dimethyl 3,3′-(but-2-yne-1,4-diyl)bis(2-(1-ethyl-3-methyl-1H-pyrazole-5-carboxamido)-3,4-dihydro-5-oxa-1,2a-diazaacenaphthylene-7-carboxylate) 6j (20 mg, 40%). MS m/z (ESI): 789 [M+1].

### Step 10 (3S,3″S)-3,3″-(but-2-yne-1,4-diyl)bis(2-(1-ethyl-3-methyl-1H-pyrazole-5-carboxamido)-3,4-dihydro-5-oxa-1,2a-diazaacenaphthylene-7-carboxylic Acid) 6k

To the MeOH solution (˜1.5 mL) of 6j was added 5N KOH aqueous solution (1.5 mL, 7.5 mmol, 30 eq), the reaction mixture was stirred at room temperature for overnight. The mixture was acidified by 6N HCl to pH<5, and the filtrated was concentrated under vacuum to give crude compound and then purified by Prep-HPLC (10-100% water: ACN (1% TFA)) to give (3S,3″S)-3,3″-(but-2-yne-1,4-diyl)bis(2-(1-ethyl-3-methyl-1H-pyrazole-5-carboxamido)-3,4-dihydro-5-oxa-1,2a-diazaacenaphthylene-7-carboxylic acid) 6k (7 mg). MS m/z (ESI): 761 [M+1]

### Step 11 (3S,3″S)-3,3″-(but-2-yne-1,4-diyl)bis(2-(1-ethyl-3-methyl-1H-pyrazole-5-carboxamido)-3,4-dihydro-5-oxa-1,2a-diazaacenaphthylene-7-carboxamide) 6

To the DMF (˜1 mL) solution of 6k (9 mg, 0.022 mmol) was added ammonium chloride (17.49 mg, 0.33 mmol, 15 eq), HATU (12.67 mg, 0.033 mmol, 1.5 eq) and TEA (9.24 uL, 0.066 mmol, 3 eq) the reaction mixture was stirred at room temperature for 2 hr. The mixture was purified by reverse phase HPLC, eluted with AcCN/H₂O/HCOOH to give (3S,3″S)-3,3″-(but-2-yne-1,4-diyl)bis(2-(1-ethyl-3-methyl-1H-pyrazole-5-carboxamido)-3,4-dihydro-5-oxa-1,2a-diazaacenaphthylene-7-carboxamide) 6 (2.5 mg). MS m/z (ESI): 759 [M+1].

### Example 7

10,10″-(but-2-ene-1,4-diyl)bis(1-(1-ethyl-3-methyl-1H-pyrazole-5-carboxamido)-7,8,9,10-tetrahydro-6-oxa-2,10a-diazacycloocta[cd]indene-4-carboxamide) 7

### Example 8

### 10-(4-((S)-7-carbamoyl-2-(1-ethyl-3-methyl-1H-pyrazole-5-carboxamido)-3,4-dihydro-5-oxa-1,2a-diazaacenaphthylen-3-yl)but-2-en-1-yl)-1-(1-ethyl-3-methyl-1H-pyrazole-5-carboxamido)-7,8,9,10-tetrahydro-6-oxa-2,10a-diazacycloocta[cd]indene-4-carboxamide 8

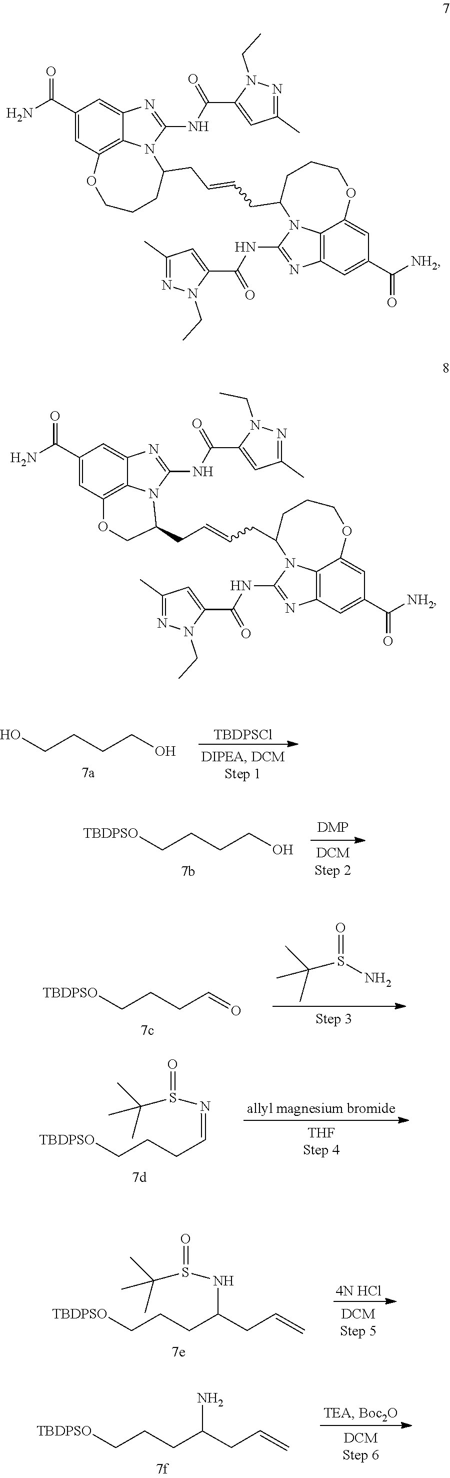

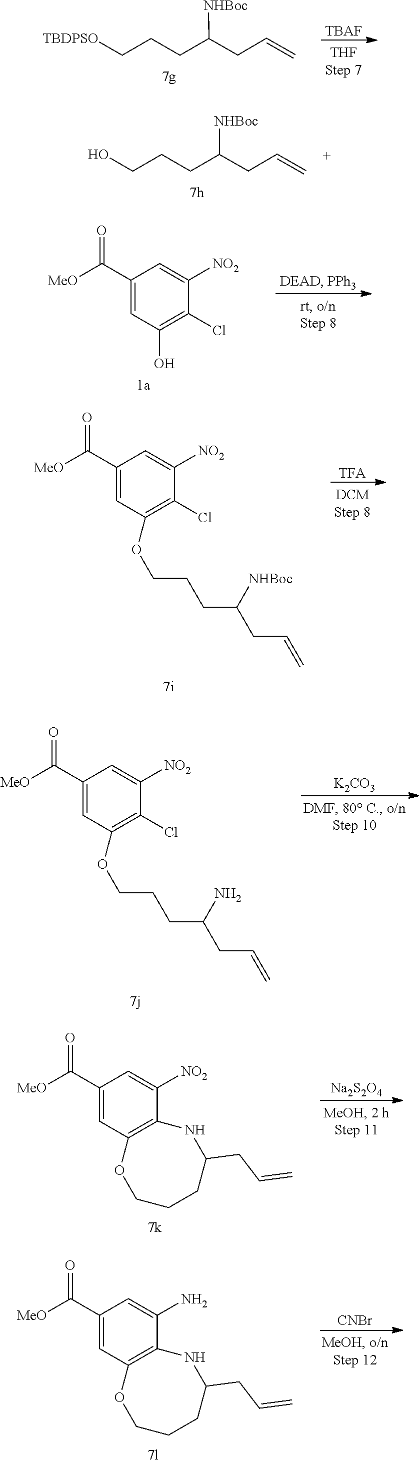

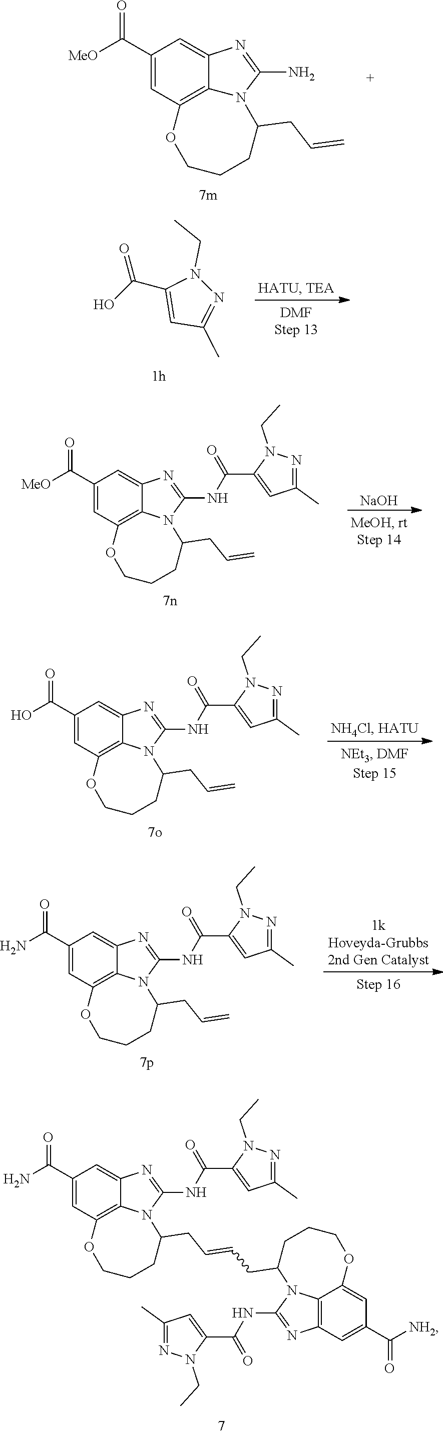

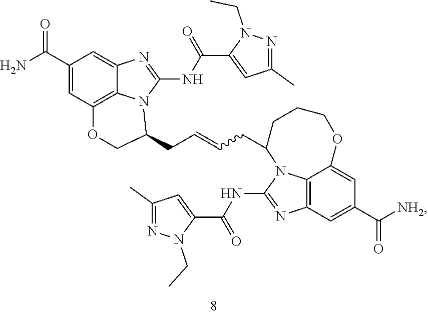 [SMILES: CCn1nc(C)cc1C(=O)Nc1nc2cc(C(N)=O)cc3c2n1C(CC=CC[C@H]1COc2cc(C(N)=O)cc4nc(NC(=O)c5cc(C)nn5CC)n1c24)CCCO3]

### Step 1 4-((tert-butyldiphenylsilyl)oxy)butan-1-ol 7b

To the dichloromethane solution (600 mL) of tert-butyl butane-1,4-diol 7a (10 g, 111 mmol) and DIPEA (21.3 mL, 122 mmol, 1.1 eq) at room temperature was added TBDPSCl (31.6 mL, 122 mmol, 1.1 eq). The resulting solution was stirred at room temperature for 72 hours.

The mixture was concentrated under vacuum and purified by silica gel column (330 g ISCO cartridge with 0-40% ethyl acetate in hexanes) to give title compound 4-((tert-butyldiphenylsilyl)oxy)butan-1-ol 7b (36 g, 98%).

### Step 2 4-((tert-butyldiphenylsilyl)oxy)butanal 7c

To the dichloromethane solution (300 mL) of 4-((tert-butyldiphenylsilyl)oxy)butan-1-ol 7b (23.5 g, 71.6 mmol) under nitrogen atmosphere at room temperature was added DMP (45.6 g, 107 mmol, 1.5 eq). The resulting solution was stirred at room temperature for 2 hours before worked up with saturated NaCl solution. After extraction with EtOAc (500 mL×3). The organic layer was combined, dried over Na₂SO₄ and filtered. The solvent was concentrated under vacuum to give title compound 4-((tert-butyldiphenylsilyl)oxy)butanal 7c, which was used in the next step without further purification.

### Step 3 N-(4-((tert-butyldiphenylsilyl)oxy)butylidene)-2-methylpropane-2-sulfinamide 7d

To the THF solution (500 mL) of 4-((tert-butyldiphenylsilyl)oxy)butanal 7c (Crude, 71.6 mmol) and 2-Methyl-2-propanesulfinamide (9.5 g, 78.8 mmol, 1.1 eq) under nitrogen atmosphere at room temperature was added Ti(OEt)₄ (27 mL, 128 mmol, 1.8 eq). The resulting solution was stirred at room temperature for 1 hour before worked up with saturated NaHCO₃ solution. After extraction with EtOAc (500 mL×3). The organic layer was combined, dried over Na₂SO₄ and filtered. The solvent was concentrated under vacuum to give title compound N-(4-((tert-butyldiphenylsilyl)oxy)butylidene)-2-methylpropane-2-sulfinamide 7d, which was used in the next step without further purification.

### Step 4 N-(7-((tert-butyldiphenylsilyl)oxy)hept-1-en-4-yl)-2-methylpropane-2-sulfinamide 7e

To the THF solution (600 mL) of N-(4-((tert-butyldiphenylsilyl)oxy)butylidene)-2-methylpropane-2-sulfinamide 7d (Crude, 71.6 mmol) under nitrogen atmosphere at −78° C. was added allyl magnesium bromide (143 mL, 143 mmol, 2 eq). The resulting solution was stirred at −78° C. for 1 hour before worked up with saturated NH₄Cl solution. After extraction with EtOAc (500 mL×3). The organic layer was combined, dried over Na₂SO₄ and filtered. The solvent was concentrated under vacuum. The resulting mixture was purified by silica gel column (2*330 g ISCO cartridge with 0-50% EtOAc in hexanes) to give title compound N-(7-((tert-butyldiphenylsilyl)oxy)hept-1-en-4-yl)-2-methylpropane-2-sulfinamide 7e (8.2 g, 24% three steps).

### Step 5 7-((tert-butyldiphenylsilyl)oxy)hept-1-en-4-amine 7f

To the DCM solution (600 mL) of N-(7-((tert-butyldiphenylsilyl)oxy)hept-1-en-4-yl)-2-methylpropane-2-sulfinamide 7e (8.2 g, 17.4 mmol) at room temperature was added 4N HCl in dioxane (13 mL, 52.2 mmol, 3 eq). The resulting solution was stirred overnight at room temperature. The solvent was concentrated under vacuum to give title compound 7-((tert-butyldiphenylsilyl)oxy)hept-1-en-4-amine 7f, which was used in the next step without further purification.

### Step 6 tert-butyl (7-((tert-butyldiphenylsilyl)oxy)hept-1-en-4-yl)azanecarboxylate 7g

To the DCM:THF solution (1:1, 300 mL) of 7-((tert-butyldiphenylsilyl)oxy)hept-1-en-4-amine 7f (Crude, 17.4 mmol) at room temperature was added NEt₃ (8.23 mL, 87 mmol, 5 eq) and Boc₂O (7.59 g, 34.8 mmol, 2 eq). The resulting solution was stirred at room temperature for 48 hours. The solvent was concentrated under vacuum. The resulting mixture was purified by silica gel column (120 g ISCO cartridge with 0-25% EtOAc in hexanes) to give title compound tert-butyl (7-((tert-butyldiphenylsilyl)oxy)hept-1-en-4-yl)-azanecarboxylate 7g (8.14 g, 98% two steps).

### Step 7 tert-butyl (7-hydroxyhept-1-en-4-yl)azanecarboxylate 7h

To the THF solution (300 mL) of 7 tert-butyl (7-((tert-butyldiphenylsilyl)oxy)hept-1-en-4-yl)azanecarboxylate 7g (8.14 g, 17.5 mmol) at room temperature was added TBAF (18.34 mL, 18.3 mmol, 1.05 eq). The resulting solution was stirred at room temperature for 6 hours. The solvent was concentrated under vacuum. The resulting mixture was purified by silica gel column (80 g ISCO cartridge with 0-100% EtOAc in hexanes) to give title compound tert-butyl (7-hydroxyhept-1-en-4-yl)azanecarboxylate 7h (1.8 g, 45%).

### Step 8-15 of Examples 7 was Prepared with the Similar Procedures as Example 1

The mixture was purified by prep-HPLC, eluated with ACN/H₂O/TFA to give title compound 10-allyl-1-(1-ethyl-3-methyl-1H-pyrazole-5-carboxamido)-7,8,9,10-tetrahydro-6-oxa-2,10a-diazacycloocta[cd]indene-4-carboxamide 7p. MS m/z (ESI): 423 [M+1]. ¹H NMR (400 MHz, Methanol-d₄): δ 7.82 (s, 1H), 7.57 (s, 1H), 6.78 (s, 1H), 5.71-5.61 (m, 2H), 4.83-4.64 (m, 3H), 3.78 (m, 1H), 3.01-2.72 (m, 3H), 2.34-2.09 (m, 7H), 1.74 (m, 1H), 1.48 (t, J=6.4 Hz, 3H).

### Step 16

To the dichloromethane/MeOH solution (1:1, 2 mL) of 10-allyl-1-(1-ethyl-3-methyl-1H-pyrazole-5-carboxamido)-7,8,9,10-tetrahydro-6-oxa-2,10a-diazacycloocta[cd]indene-4-carboxamide 7p (10 mg) and (S)-3-allyl-2-(1-ethyl-3-methyl-1H-pyrazole-5-carboxamido)-3,4-dihydro-5-oxa-1,2a-diazaacenaphthylene-7-carboxamide 1k (7 mg) at room temperature was added TsOH.H₂O (15 mg) in MeOH (0.5 mL). The resulting solution was stirred at room temperature for 15 min and then concentrated under vacuum. To re-dissolved residue in DCM (2 mL) under N₂ was added Hoveyda-Grubbs 2nd Gen Catalyst (15 mg). The resulting solution was stirred 3 hours at 80° C. The mixture was concentrated, and then purified by prep-HPLC, eluted with ACN/H₂O/NH₄HCO₃ to give title compounds:

### Example 7

(Shorter retention time on reverse phase HPLC), 10,10″-(but-2-ene-1,4-diyl)bis(1-(1-ethyl-3-methyl-1H-pyrazole-5-carboxamido)-7,8,9,10-tetrahydro-6-oxa-2,10a-diazacycloocta[cd]indene-4-carboxamide) 7 (1.8 mg, 12%) with MS m/z (ESI): 817 [M+1], 815 [M−1].

### Example 8

(Longer retention time on reverse phase HPLC), 10-(4-((S)-7-carbamoyl-2-(1-ethyl-3-methyl-1H-pyrazole-5-carboxamido)-3,4-dihydro-5-oxa-1,2a-diazaacenaphthylen-3-yl)but-2-en-1-yl)-1-(1-ethyl-3-methyl-1H-pyrazole-5-carboxamido)-7,8,9,10-tetrahydro-6-oxa-2,10a-diazacycloocta[cd]indene-4-carboxamide 8 (1.4 mg, 10%) with MS m/z (ESI): 789 [M+1], 787[M−1].

### Example 9

### (4S,4′S)-4,4′-((E)-but-2-ene-1,4-diyl)bis(2-(1-ethyl-3-methyl-1H-pyrazole-5-carboxamido)-6-methyl-5,6-dihydro-4H-imidazo[1,5,4-de]quinoxaline-8-carboxamide) 9

### Example 10

### (4S,4′S)-4,4′-((Z)-but-2-ene-1,4-diyl)bis(2-(1-ethyl-3-methyl-1H-pyrazole-5-carboxamido)-6-methyl-5,6-dihydro-4H-imidazo[1,5,4-de]quinoxaline-8-carboxamide) 10

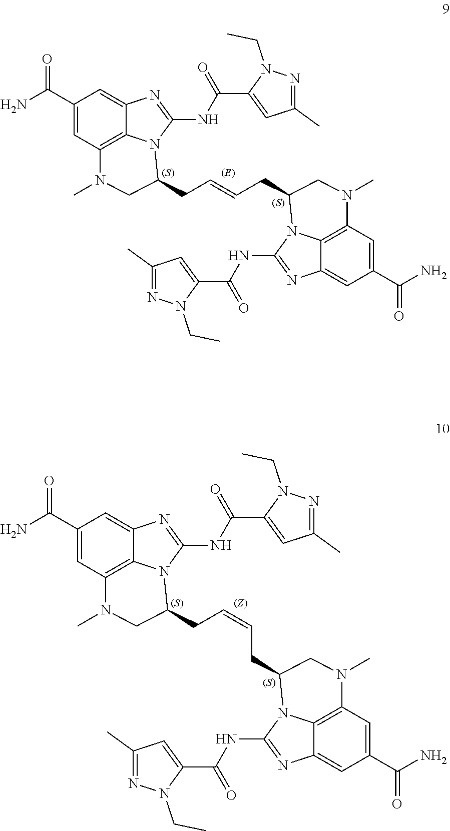

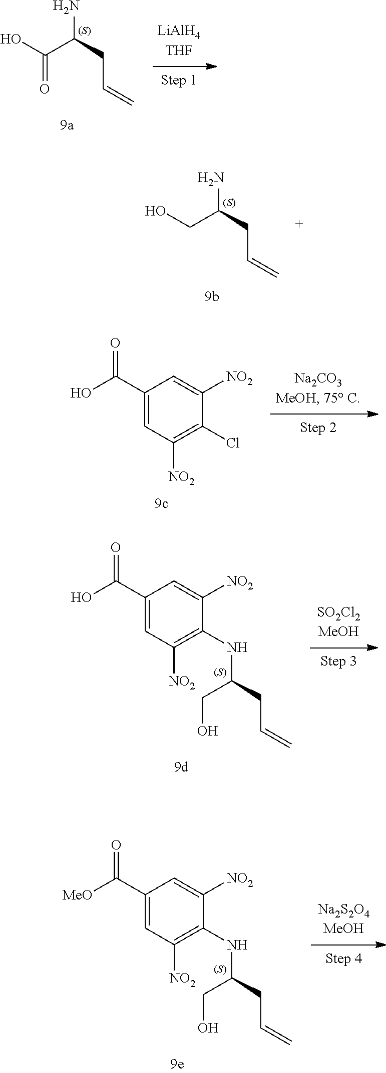

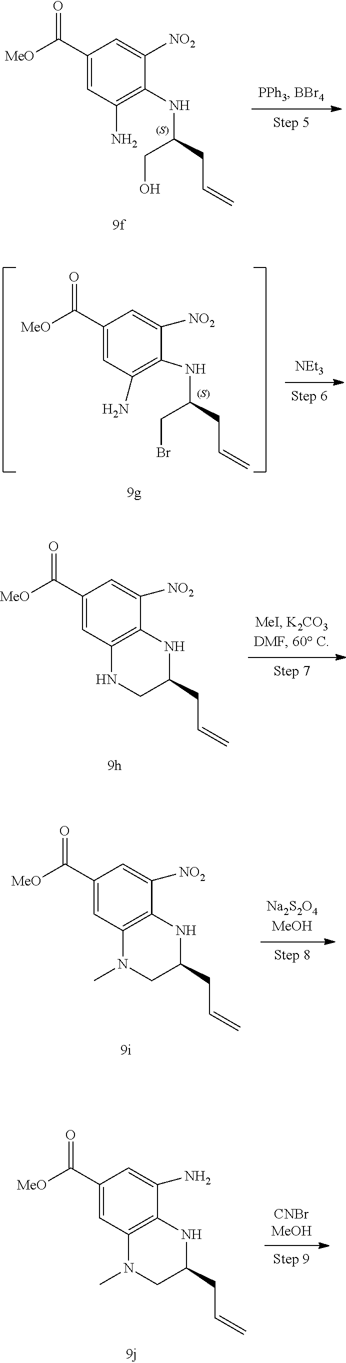

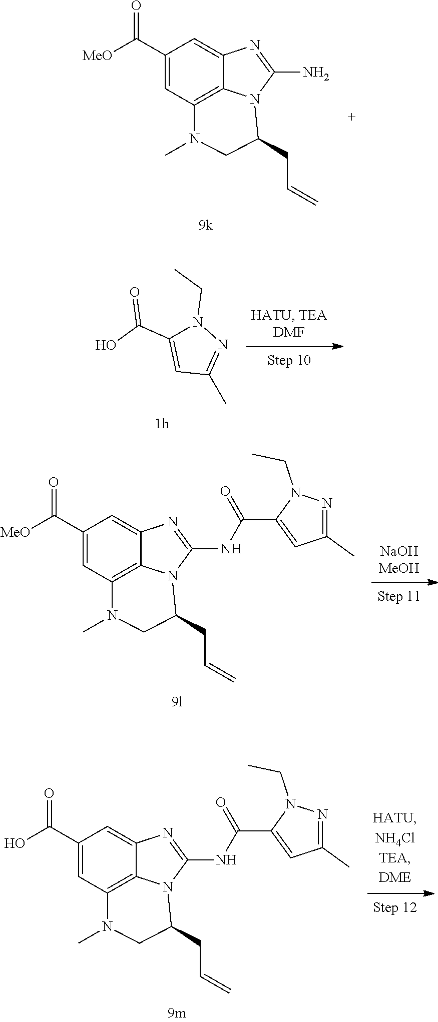 [SMILES: C=CC[C@H]1CN(C)c2cc(C(=O)OC)cc3nc(N)n1c23 ; CCn1nc(C)cc1C(=O)O ; C=CC[C@H]1CN(C)c2cc(C(=O)OC)cc3nc(NC(=O)c4cc(C)nn4CC)n1c23 ; C=CC[C@H]1CN(C)c2cc(C(=O)O)cc3nc(NC(=O)c4cc(C)nn4CC)n1c23]

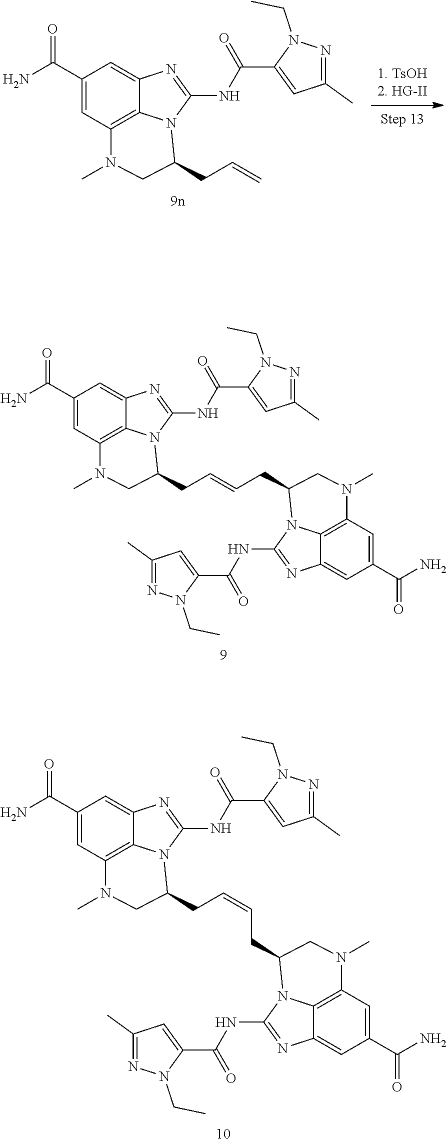 [SMILES: C=CC[C@H]1CN(C)c2cc(C(N)=O)cc3nc(NC(=O)c4cc(C)nn4CC)n1c23 ; CCn1nc(C)cc1C(=O)Nc1nc2cc(C(N)=O)cc3c2n1[C@@H](C/C=C/C[C@H]1CN(C)c2cc(C(N)=O)cc4nc(NC(=O)c5cc(C)nn5CC)n1c24)CN3C ; CCn1nc(C)cc1C(=O)Nc1nc2cc(C(N)=O)cc3c2n1[C@@H](C/C=C\C[C@H]1CN(C)c2cc(C(N)=O)cc4nc(NC(=O)c5cc(C)nn5CC)n1c24)CN3C]

### Step 1(S)-2-aminopent-4-en-1-ol 9b

To the THF solution (300 mL) of (S)-2-aminopent-4-enoic acid 9a (10 g, 86.7 mmol) under nitrogen atmosphere at room temperature was LiAlH₄ (108 mL, 1M in THF). The resulting solution was stirred overnight room temperature before quenching by addition of MeOH at 0° C. The mixture was diluted with brine (500 mL), extracted with EtOAc (500 mL×3). The organic layer was combined, dried over Na₂SO₄ and filtered. The solvent was concentrated under vacuum to give (S)-2-aminopent-4-en-1-ol 9b, which was used in the next step without further purification.

### Step 2 (S)-4-((1-hydroxypent-4-en-2-yl)amino)-3,5-dinitrobenzoic acid 9d

To the water solution (100 mL) of methyl (S)-2-aminopent-4-en-1-ol 9b (Crude from above, 86.9 mmol) and Na₂CO₃ (13.6 g, 129 mmol) at 75° C. was added 4-chloro-3,5-dinitrobenzoic acid 9c (21 g, 86.9 mmol). The resulting solution was stirred at 75° C. for 2 hours. After cooling down, the mixture was concentrated. The resulting mixture was purified by silica gel column (2*330 g ISCO cartridge with 0-100% MeOH in DCM) to give title compound (S)-4-((1-hydroxypent-4-en-2-yl)amino)-3,5-dinitrobenzoic acid 9d (>100%, contain silica gel).

### Step 3 (S)-methyl 4-((1-hydroxypent-4-en-2-yl)amino)-3,5-dinitrobenzoate 9e

To the MeOH solution (800 mL) of compound 9d (Crude, 86.7 mmol, 1 eq) at 0° C. was added SOCl₂ (10 mL, catalytic amount). The resulting solution was slowly warm up to 75° C. and stirred for 2 hours. The mixture was cooled to room temperature before concentrated under vacuum, and purified by silica gel column (2*330 g ISCO cartridge with 0-100% hexanes: EtOAc) to give title compound (S)-methyl 4-((1-hydroxypent-4-en-2-yl)amino)-3,5-dinitrobenzoate 9e (9.2 g, 32% three steps).

Step 4 of Example 9 was prepared with the similar procedures as Example 1.

### Step 5 and Step 6 (S)-methyl 2-allyl-8-nitro-1,2,3,4-tetrahydroquinoxaline-6-carboxylate 9h

To the MeCN solution (40 mL) of methyl (S)-3-amino-4-((1-hydroxypent-4-en-2-yl)amino)-5-nitrobenzoate 9f (1.2 g, 4.06 mmol) and PPh₃ (2.34 g, 8.95 mmol, 2.2 eq) under nitrogen atmosphere at room temperature was added CBr₄ (3 g, 8.95 mmol, 2.2 eq) in MeCN (10 mL). The resulting solution was stirred at room temperature for 15 min before addition of NEt₃ (1.7 mL, 17.8 mmol, 4.4 eq). After stirring 30 min at room temperature, the mixture was concentrated. The resulting mixture was purified by silica gel column (40 g ISCO cartridge with 0-100% with 0-100% EtOAc in Hexanes) to give title compound (S)-methyl 2-allyl-8-nitro-1,2,3,4-tetrahydroquinoxaline-6-carboxylate 9h (872 mg, 77%).

### Step 7 (S)-methyl 2-allyl-4-methyl-8-nitro-1,2,3,4-tetrahydroquinoxaline-6-carboxylate 9i

To the DMF solution (10 mL) of (S)-methyl 2-allyl-8-nitro-1,2,3,4-tetrahydroquinoxaline-6-carboxylate 9h (340 mg, 1.23 mmol) and K₂CO₃ (338 mg, 2.45 mmol, 2 eq) under nitrogen atmosphere at 60° C. was added Mel (1.5 mL). The resulting solution was stirred at 60° C. for 45 min before adding more Mel (1 mL). The mixture was stirred for another 30 min before cooling down and concentrated under vacuum. The residue was purified by silica gel column (20 g ISCO cartridge with 0-100% EtOAc in DCM) to give title compound (S)-methyl 2-allyl-4-methyl-8-nitro-1,2,3,4-tetrahydroquinoxaline-6-carboxylate 9i and its isomer (300 mg, 84%).

Steps 8-12 of Example 9 were prepared with the similar procedures as Example 1.

In step 12, the mixture was purified by prep-HPLC, eluated with ACN/H₂O/formic acid to give title compound (S)-4-allyl-2-(1-ethyl-3-methyl-1H-pyrazole-5-carboxamido)-6-methyl-5,6-dihydro-4H-imidazo[1,5,4-de]quinoxaline-8-carboxamide 9n. MS m/z (ESI): 408 [M+1]. 1H NMR (400 MHz, Methanol-d₄): δ 7.42 (s, 1H), 7.12 (s, 1H), 6.71 (s, 1H), 6.05-5.96 (m, 1H), 5.15-5.12 (m, 2H), 4.76-4.68 (m, 3H), 3.56-3.37 (m, 1H), 3.36-3.33 (m, 1H), 3.33 (s, 3H), 2.70-2.61 (m, 2H), 2.27 (s, 3H), 1.45 (t, J=7.2 Hz, 3H).

Step 13 of Examples 9 and 10 was prepared with the similar procedures as Example 7 and 8.

To the dichloromethane/MeOH solution (1:1, 2 mL) of (S)-4-allyl-2-(1-ethyl-3-methyl-1H-pyrazole-5-carboxamido)-6-methyl-5,6-dihydro-4H-imidazo[1,5,4-de]quinoxaline-8-carboxamide 9n (15 mg) at room temperature was added TsOH.H₂O (12 mg) in MeOH (0.5 mL). The resulting solution was stirred at room temperature for 15 min and then concentrated under vacuum. To re-dissolved residue in DCM (2 mL) under N₂ was added Hoveyda-Grubbs 2nd Gen Catalyst (15 mg). The resulting solution was stirred 1 hour at 80° C. After the reaction was done, the mixture was concentrated, and then purified by prep-HPLC, eluted with ACN/H₂O/TFA. The first elute was title compound (4S,4′S)-4,4′-((E)-but-2-ene-1,4-diyl)bis(2-(1-ethyl-3-methyl-1H-pyrazole-5-carboxamido)-6-methyl-5,6-dihydro-4H-imidazo[1,5,4-de]quinoxaline-8-carboxamide) 9 (1.8 mg, 12%). MS m/z (ESI): 787 [M+1]. ¹H NMR (400 MHz, Methanol-d₄): δ 7.31 (m, 2H), 6.98 (m, 2H), 6.48 (m, 2H), 6.60-5.48 (m, 2H), 5.15-5.12 (m, 4H), 4.67-4.64 (m, 2H), 4.54-4.47 (m, 2H), 4.41-4.33 (m, 2H), 2.89 (s, 6H), 2.45-2.37 (m, 4H), 2.18 (s, 6H), 1.21 (t, J=7.2 Hz, 6H).

The second elute was title compound (4S,4′S)-4,4′-((Z)-but-2-ene-1,4-diyl)bis(2-(1-ethyl-3-methyl-1H-pyrazole-5-carboxamido)-6-methyl-5,6-dihydro-4H-imidazo[1,5,4-de]quinoxaline-8-carboxamide) 10 (2.5 mg, 17%). MS m/z (ESI): 787 [M+1] 785 [M−1]. ¹H NMR (400 MHz, Methanol-d₄): δ 7.20 (m, 2H), 6.92 (m, 2H), 6.33 (m, 2H), 5.73-5.71 (m, 2H), 5.15-5.12 (m, 4H), 4.74-4.67 (m, 2H), 4.49-4.39 (m, 4H), 2.61 (s, 6H), 2.58-2.38 (m, 2H), 2.19-2.16 (m, 2H), 1.69 (s, 6H), 1.30 (t, J=7.1 Hz, 6H).

### Example 11

### (4S,4′S)-4,4′-((E)-but-2-ene-1,4-diyl)bis(2-(1-ethyl-3-methyl-1H-pyrazole-5-carboxamido)-6-(3-methoxypropyl)-5,6-dihydro-4H-imidazo[1,5,4-de]quinoxaline-8-carboxamide) 11

### Example 12

### (4S,4′S)-4,4′-((Z)-but-2-ene-1,4-diyl)bis(2-(1-ethyl-3-methyl-1H-pyrazole-5-carboxamido)-6-(3-methoxypropyl)-5,6-dihydro-4H-imidazo[1,5,4-de]quinoxaline-8-carboxamide) 12

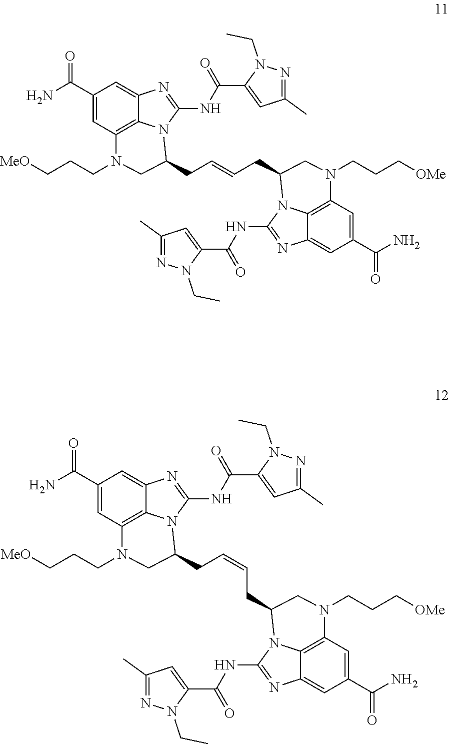 [SMILES: CCn1nc(C)cc1C(=O)Nc1nc2cc(C(N)=O)cc3c2n1[C@@H](C/C=C/C[C@H]1CN(CCCOC)c2cc(C(N)=O)cc4nc(NC(=O)c5cc(C)nn5CC)n1c24)CN3CCCOC ; CCn1nc(C)cc1C(=O)Nc1nc2cc(C(N)=O)cc3c2n1[C@@H](C/C=C\C[C@H]1CN(CCCOC)c2cc(C(N)=O)cc4nc(NC(=O)c5cc(C)nn5CC)n1c24)CN3CCCOC]

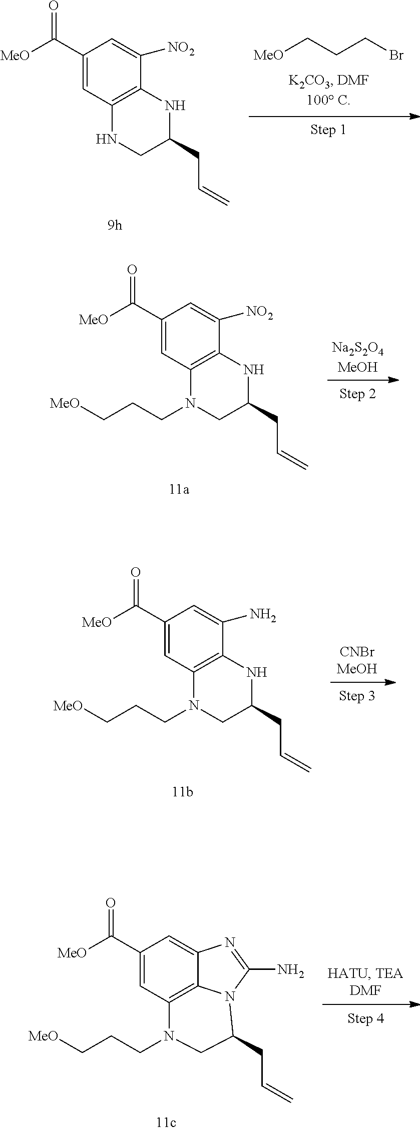 [SMILES: C=CC[C@H]1CNc2cc(C(=O)OC)cc([N+](=O)[O-])c2N1 ; COCCCBr ; C=CC[C@H]1CN(CCCOC)c2cc(C(=O)OC)cc([N+](=O)[O-])c2N1 ; C=CC[C@H]1CN(CCCOC)c2cc(C(=O)OC)cc(N)c2N1 ; C=CC[C@H]1CN(CCCOC)c2cc(C(=O)OC)cc3nc(N)n1c23]

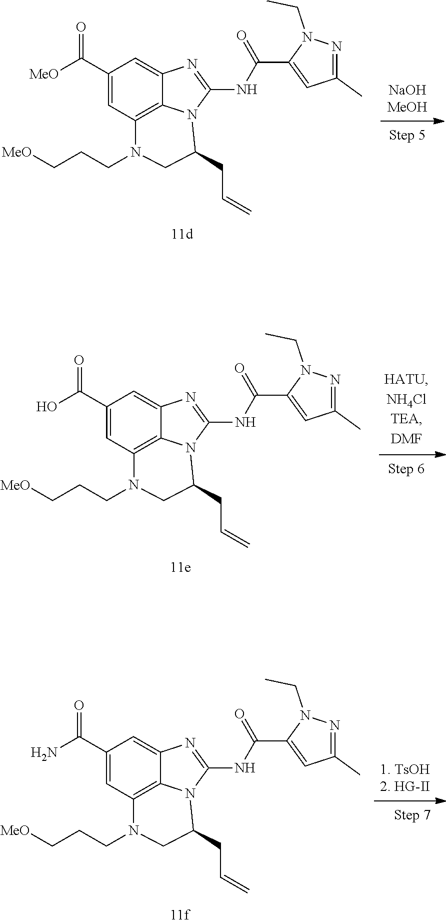 [SMILES: C=CC[C@H]1CN(CCCOC)c2cc(C(=O)OC)cc3nc(NC(=O)c4cc(C)nn4CC)n1c23 ; C=CC[C@H]1CN(CCCOC)c2cc(C(=O)O)cc3nc(NC(=O)c4cc(C)nn4CC)n1c23 ; C=CC[C@H]1CN(CCCOC)c2cc(C(N)=O)cc3nc(NC(=O)c4cc(C)nn4CC)n1c23]

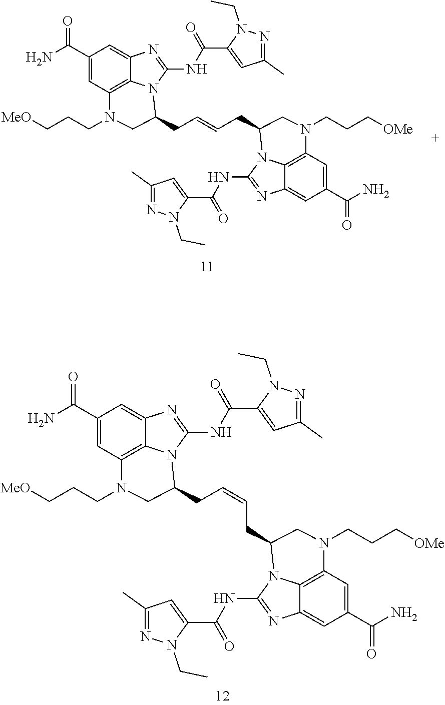 [SMILES: CCn1nc(C)cc1C(=O)Nc1nc2cc(C(N)=O)cc3c2n1[C@@H](C/C=C/C[C@H]1CN(CCCOC)c2cc(C(N)=O)cc4nc(NC(=O)c5cc(C)nn5CC)n1c24)CN3CCCOC ; CCn1nc(C)cc1C(=O)Nc1nc2cc(C(N)=O)cc3c2n1[C@@H](C/C=C\C[C@H]1CN(CCCOC)c2cc(C(N)=O)cc4nc(NC(=O)c5cc(C)nn5CC)n1c24)CN3CCCOC]

### Step 1 (S)-methyl 2-allyl-4-(3-methoxypropyl)-8-nitro-1,2,3,4-tetrahydroquinoxaline-6-carboxylate 11a

To the DMF solution (5 mL) of methyl (S)-2-allyl-8-nitro-1,2,3,4-tetrahydroquinoxaline-6-carboxylate 9h (97 mg, 0.35 mmol) and K₂CO₃ (97 mg, 0.70 mmol, 2 eq) under nitrogen atmosphere at 100° C. was added 1-bromo-3-methoxypropane (2 mL). The resulting solution was stirred overnight at 100° C. The mixture was concentrated under vacuum and purified by silica gel column (20 g ISCO cartridge with 0-100% EtOAc in Hexanes) to give title compound (S)-2-allyl-4-(3-methoxypropyl)-8-nitro-1,2,3,4-tetrahydroquinoxaline-6-carboxylate 11a and its regioisomer (100 mg, 81%).

Steps 2-6 of Examples 11 and 12 were prepared with the similar procedures as in Example 1.

### Step 7 (4S,4′S)-4,4′-((E)-but-2-ene-1,4-diyl)bis(2-(1-ethyl-3-methyl-1H-pyrazole-5-carboxamido)-6-(3-methoxypropyl)-5,6-dihydro-4H-imidazo[1,5,4-de]quinoxaline-8-carboxamide) 11

### (4S,4′S)-4,4′-((Z)-but-2-ene-1,4-diyl)bis(2-(1-ethyl-3-methyl-1H-pyrazole-5-carboxamido)-6-(3-methoxypropyl)-5,6-dihydro-4H-imidazo[1,5,4-de]quinoxaline-8-carboxamide) 12

To the dichloromethane/MeOH solution (1:1, 2 mL) of (S)-4-allyl-2-(1-ethyl-3-methyl-1H-pyrazole-5-carboxamido)-6-(3-methoxypropyl)-5,6-dihydro-4H-imidazo[1,5,4-de]quinoxaline-8-carboxamide 11f (15 mg, at room temperature was added TsOH.H₂O (17 mg) in MeOH (1 mL). The resulting solution was stirred at room temperature for 20 min and then concentrated under vacuum. To re-dissolved residue in DCM (2 mL) under N₂ was added Hoveyda-Grubbs 2nd Gen Catalyst (15 mg). The resulting solution was stirred 2 hours at 80° C. After the reaction was done, the mixture was concentrated, and then purified by prep-HPLC, eluted with ACN/H₂O/formic acid. The first elute was title compound (4S,4′S)-4,4′-((E)-but-2-ene-1,4-diyl)bis(2-(1-ethyl-3-methyl-1H-pyrazole-5-carboxamido)-6-(3-methoxypropyl)-5,6-dihydro-4H-imidazo[1,5,4-de]quinoxaline-8-carboxamide) 11 (1.3 mg, yield 9%). MS m/z (ESI): 903 [M+1] 901 [M−1].

The second elute was title compound (4S,4′S)-4,4′-((Z)-but-2-ene-1,4-diyl)bis(2-(1-ethyl-3-methyl-1H-pyrazole-5-carboxamido)-6-(3-methoxypropyl)-5,6-dihydro-4H-imidazo[1,5,4-de]quinoxaline-8-carboxamide) 12 (2.1 mg, yield 15%). MS m/z (ESI): 903 [M+1] 901 [M−1].

### Example 13

### (S)-4-((E)-4-((S)-8-carbamoyl-2-(I-ethyl-3-methyl-1H-pyrazole-5-carboxamido)-6-(3-methoxypropyl)-5,6-dihydro-4H-imidazo[1,5,4-de]quinoxalin-4-yl)but-2-en-1-yl)-2-(1-ethyl-3-methyl-1H-pyrazole-5-carboxamido)-6-methyl-5,6-dihydro-4H-imidazo[1,5,4-de]quinoxaline-8-carboxamide 13

### Example 14

### (S)-4-((Z)-4-((S)-8-carbamoyl-2-(1-ethyl-3-methyl-1H-pyrazole-5-carboxamido)-6-(3-methoxypropyl)-5,6-dihydro-4H-imidazo[1,5,4-de]quinoxalin-4-yl)but-2-en-1-yl)-2-(1-ethyl-3-methyl-1H-pyrazole-5-carboxamido)-6-methyl-5,6-dihydro-4H-imidazo[1,5,4-de]quinoxaline-8-carboxamide 14

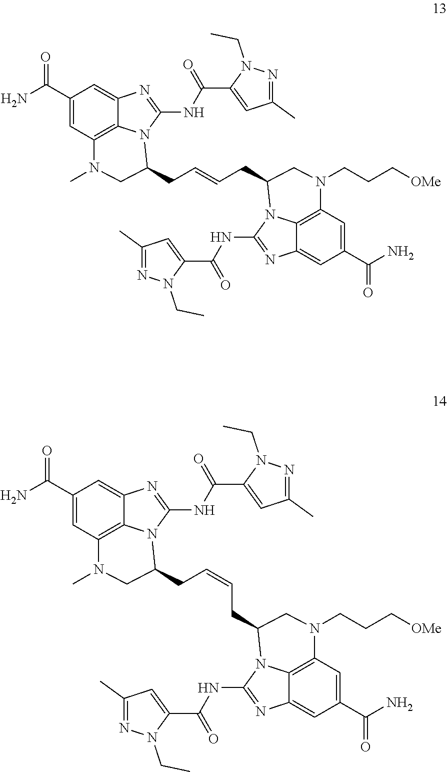 [SMILES: CCn1nc(C)cc1C(=O)Nc1nc2cc(C(N)=O)cc3c2n1[C@@H](C/C=C/C[C@H]1CN(CCCOC)c2cc(C(N)=O)cc4nc(NC(=O)c5cc(C)nn5CC)n1c24)CN3C ; CCn1nc(C)cc1C(=O)Nc1nc2cc(C(N)=O)cc3c2n1[C@@H](C/C=C\C[C@H]1CN(CCCOC)c2cc(C(N)=O)cc4nc(NC(=O)c5cc(C)nn5CC)n1c24)CN3C]

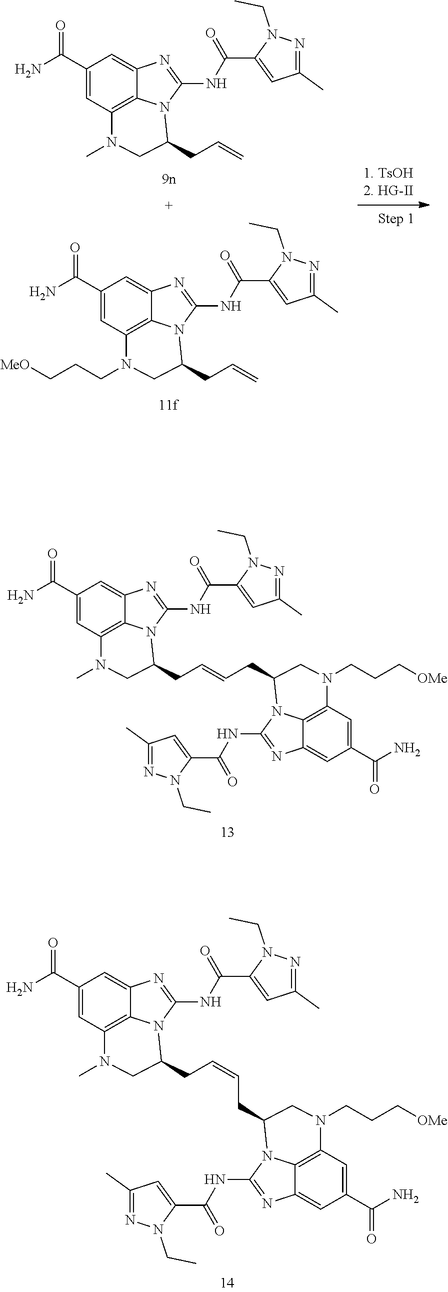 [SMILES: C=CC[C@H]1CN(C)c2cc(C(N)=O)cc3nc(NC(=O)c4cc(C)nn4CC)n1c23 ; C=CC[C@H]1CN(CCCOC)c2cc(C(N)=O)cc3nc(NC(=O)c4cc(C)nn4CC)n1c23 ; CCn1nc(C)cc1C(=O)Nc1nc2cc(C(N)=O)cc3c2n1[C@@H](C/C=C/C[C@H]1CN(CCCOC)c2cc(C(N)=O)cc4nc(NC(=O)c5cc(C)nn5CC)n1c24)CN3C ; CCn1nc(C)cc1C(=O)Nc1nc2cc(C(N)=O)cc3c2n1[C@@H](C/C=C\C[C@H]1CN(CCCOC)c2cc(C(N)=O)cc4nc(NC(=O)c5cc(C)nn5CC)n1c24)CN3C]

Step 1 of Examples 13 and 14 was prepared with the similar procedures as Example 7 and 8

To the dichloromethane/MeOH solution (1:1, 4 mL) of (S)-4-allyl-2-(1-ethyl-3-methyl-1H-pyrazole-5-carboxamido)-6-methyl-5,6-dihydro-4H-imidazo[1,5,4-de]quinoxaline-8-carboxamide 9n (25 mg) and (S)-4-allyl-2-(1-ethyl-3-methyl-1H-pyrazole-5-carboxamido)-6-(3-methoxypropyl)-5,6-dihydro-4H-imidazo[1,5,4-de]quinoxaline-8-carboxamide 11f (29 mg) at room temperature was added TsOH.H₂O (60 mg) in MeOH (2 mL). The resulting solution was stirred at room temperature for 20 min and then concentrated under vacuum. To re-dissolved residue in DCM (4 mL) under N₂ was added Hoveyda-Grubbs 2nd Gen Catalyst (25 mg). After the reaction was done, the mixture was concentrated, and then purified by prep-HPLC, eluted with ACN/H₂O/formic acid. The first elute was title compound (S)-4-((E)-4-((S)-8-carbamoyl-2-(1-ethyl-3-methyl-1H-pyrazole-5-carboxamido)-6-(3-methoxypropyl)-5,6-dihydro-4H-imidazo[1,5,4-de]quinoxalin-4-yl)but-2-en-1-yl)-2-(1-ethyl-3-methyl-1H-pyrazole-5-carboxamido)-6-methyl-5,6-dihydro-4H-imidazo[1,5,4-de]quinoxaline-8-carboxamide 13 (1.9 mg, 4%). MS m/z (ESI): 845 [M+1] 843 [M−1].

The second elute was title compound (S)-4-((Z)-4-((S)-8-carbamoyl-2-(1-ethyl-3-methyl-1H-pyrazole-5-carboxamido)-6-(3-methoxypropyl)-5,6-dihydro-4H-imidazo[1,5,4-de]quinoxalin-4-yl)but-2-en-1-yl)-2-(1-ethyl-3-methyl-1H-pyrazole-5-carboxamido)-6-methyl-5,6-dihydro-4H-imidazo[1,5,4-de]quinoxaline-8-carboxamide 14 (4.4 mg, 8%). MS m/z (ESI): 845 [M+1] 843 [M−1].

The compounds 15-20 can be prepared with the similar procedures as illustrated in Examples 1-14.

### Example 15

### (3S,3″S)-3,3″-(cyclopropane-1,2-diylbis(methylene))bis(2-(1-ethyl-3-methyl-1H-pyrazole-5-carboxamido)-3,4-dihydro-5-oxa-1,2a-diazaacenaphthylene-7-carboxamide) 15

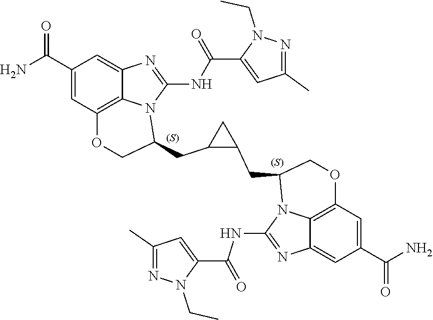

### Example 16

### (3S,3″S)-3,3″-(2,3-dihydroxybutane-1,4-diyl)bis(2-(1-ethyl-3-methyl-1H-pyrazole-5-carboxamido)-3,4-dihydro-5-oxa-1,2a-diazaacenaphthylene-7-carboxamide) 16

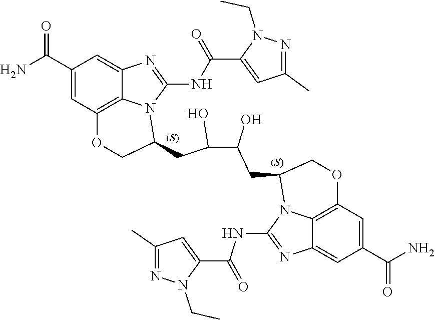

### Example 17

### 3,3″-(prop-1-ene-1,3-diyl)bis(2-(1-ethyl-3-methyl-1H-pyrazole-5-carboxamido)-3,4-dihydro-5-oxa-1,2a-diazaacenaphthylene-7-carboxamide) 17

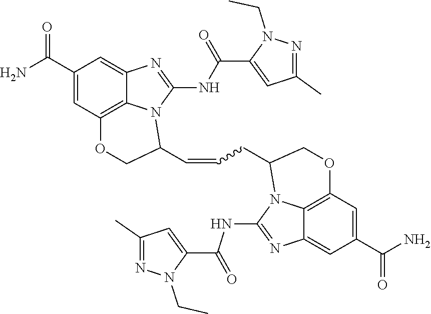 [SMILES: CCn1nc(C)cc1C(=O)Nc1nc2cc(C(N)=O)cc3c2n1C(C=CCC1COc2cc(C(N)=O)cc4nc(NC(=O)c5cc(C)nn5CC)n1c24)CO3]

### Example 18

### 3,3″-(propane-1,3-diyl)bis(2-(1-ethyl-3-methyl-1H-pyrazole-5-carboxamido)-3,4-dihydro-5-oxa-1,2a-diazaacenaphthylene-7-carboxamide) 18

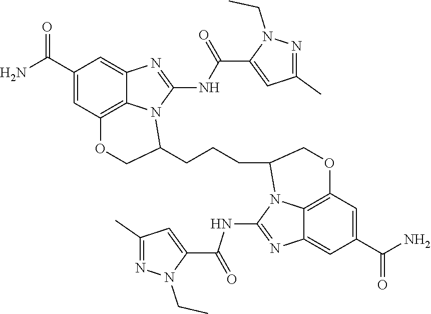 [SMILES: CCn1nc(C)cc1C(=O)Nc1nc2cc(C(N)=O)cc3c2n1C(CCCC1COc2cc(C(N)=O)cc4nc(NC(=O)c5cc(C)nn5CC)n1c24)CO3]

### Example 19

### (3S,3″S)-3,3″-(pent-2-ene-1,5-diyl)bis(2-(1-ethyl-3-methyl-1H-pyrazole-5-carboxamido)-3,4-dihydro-5-oxa-1,2a-diazaacenaphthylene-7-carboxamide) 19

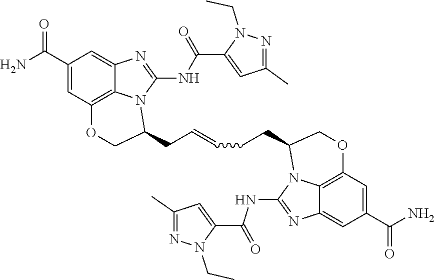 [SMILES: CCn1nc(C)cc1C(=O)Nc1nc2cc(C(N)=O)cc3c2n1[C@@H](CC=CCC[C@H]1COc2cc(C(N)=O)cc4nc(NC(=O)c5cc(C)nn5CC)n1c24)CO3]

### Example 20

### (3S,3″S)-3,3″-(1,2-phenylenebis(methylene))bis(2-(1-ethyl-3-methyl-1H-pyrazole-5-carboxamido)-3,4-dihydro-5-oxa-1,2a-diazaacenaphthylene-7-carboxamide) 20

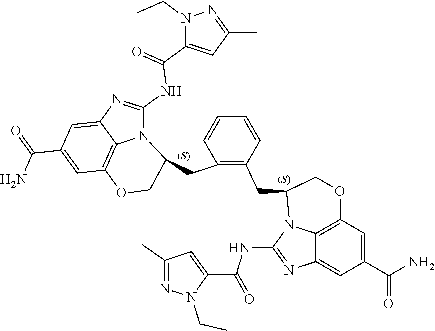

### Biological Assays

### Test Example 1

Thermal shift assay for measuring the relative binding affinity to hSTING R232 c-terminal domain.

### Materials and Reagents

1. SYPRO Orange Stain (Thermo Fisher Scientific)

2. Buffer—20 mM HEPES pH 7.2, 150 mM NaCl (Sigma)

3. DMSO (Thermo Fisher Scientific)

4. Purified hSTING R232 (aa154-342)

5. Compounds—10 mM stock in DMSO

6. cGAMP—10 mM stock in DMSO (Sigma)

7. Light Cycler 480 II (Roche)

8. Light Cycler 480 Multi-well Plates, 384-well white (Roche)

### Experimental Procedure

From each of the 10 mM stock solutions of compounds in DMSO, dilutions are made to create samples with three concentrations 10 mM, 5 mM, and 2.5 mM. From these dilutions a final 50-fold dilution is made into assay buffer, giving concentrations of 200 μM, 100 μM, and 50 μM. From each of the buffer dilutions, 5 μL is added to the 384 well assay plate. A positive control is setup with cGAMP using the same dilution scheme as the ligands. A baseline thermal shift for the negative control is determined using buffer and 2% DMSO.

An aliquot of protein is thawed on ice and SYPRO orange reagent is brought to room temperature. The 5000×SYPRO orange stock is diluted in assay buffer to a concentration of 10×. Protein is diluted to a concentration of 10 μM in the prepared buffer/dye solution. Five micro-liters of protein/buffer/dye solution is added to each of the sample and control wells, and the plate is sealed with the provided films. The plate is centrifuged for 5 min at 20° C. at 1000 rpm.

On the Light Cycler instrument, measurements are made over a temperature gradient from 20° C. to 99° C. at 0.07° C./s and data acquisitions collected at a rate of 8/° C. are used to measure the change in fluorescence as a function of temperature. Data analysis is performed using the Roche Light Cycler Software to determine the melting temperature (Tm ° C.) of each sample. A mean Tm ° C. for the negative control is calculated and subtracted from each of the samples to generate the ΔTm ° C. values for each of the ligands.

The relative binding affinity to hSTING c-terminal domain of the compounds of the present invention was determined by the above assay, and ΔTm ° C. values are shown in the following Table 3.

TABLE 3
Thermal Shift ΔTm ° C. values of the compounds in the present invention.

| Example No. | Thermal Shift ΔTm (° C.) at 100 μM |
|---|---|
| 1 | 14.6 |
| 2 | 12.2 |
| 3 | 5.9 |
| 4 | 7.5 |
| 5 | 9.1 |
| 7 | 6.2 |

Conclusion: The compounds of the present invention showed binding affinity to a human STING protein.

### Test Example 2. Human THP1 Reporter Cell Assay

### Materials and Reagents

1. Human THP1-Dual KI-hSTING-R232 Cells (InvivoGen, Cat. #thpd-r232)

2. QUANTI-LUC (InvivoGen, Cat. #rep-qlc2)

3. Media for cell culture and compound dilution: RPMI with high glucose and glutamine (Genesee, Cat. #25-506), 10% fetal bovine serum (Life Technologies, Cat. #10082147), 25 mM HEPES (Genesee, Cat. #25-534), 100 μg/ml Normocin (InvivoGen, Cat. #ant-nr-2), 10 μg/ml blasticidin (InvivoGen, Cat. #ant-bl-05), 100 μg/ml Zeocin (InvivoGen, Cat. #ant-zn-5p) and Penicillin-Streptomycin (100X) (Life Technologies, Cat. #15140122) 4. Infinite M1000 plate reader (TECAN)

### Experimental Procedure

Activation of STING in THP1-Dual KI-hSTING-R232 cells was determined by measuring the luminescence signal resulting from the expression of the IRF luciferase reporter gene. All reagent preparation and assay procedures were conducted according to the protocols provided by InvivoGen. In brief, test compounds and cells (1×10⁵ cells per well) were dispensed into 96-well plates with a final volume per well of 150 μl. Plates were incubated in a humidified, 5% CO₂ incubator at 37° C. for 24 hours. The expression level of the reporter gene was measured by transferring 20 μl of the supernatant to a non-transparent 96-well plate to which 50 μl of QUANTI-LUC was dispensed into each well. The resulting luminescence signal was immediately read using a TECAN plate reader. The background luminescence signal from media was subtracted. The fold induction effect of the luminescence signal at each compound concentration was determined relative to controls that lack compound treatment. The plot of fold induction effect versus the log of compound concentration was fit in GraphPad Prism with a 4-parameter concentration response equation to calculate EC₅₀ and Emax.

Activation of STING in THP1 of the compounds in the present invention was determined by the above assay, and EC₅₀ values are shown in the following Table 4.

TABLE 4
Human THP1 reporter cell assay

| Example No | EC₅₀ (THP1 R232, μM) |
|---|---|
| 1 | 0.2 |
| 2 | 0.006 |
| 4 | 11.5 |
| 5 | 0.43 |
| 8 | 6.4 |
| 9 | 0.054 |
| 10 | 0.012 |
| 11 | 0.15 |
| 12 | 0.14 |
| 13 | 0.18 |
| 14 | 0.06 |

Conclusion: The compounds of the present invention had significant stimulatory activity on human STING.

### Test Example 3. IFNβ Secretion from Human PBMC

### Materials and Reagents

1. Human PBMC cells (STEMCELL Technologies)

2. Lymphocyte Medium (Zenbio)

3. Culture and compound dilution media: RPMI with high glucose and glutamine (Genesee, Cat. #25-506), 10% fetal bovine serum (Life Technologies, Cat. #10082147), 100 μg/ml Normocin (InvivoGen, Cat. #ant-nr-2) and Penicillin-Streptomycin (100×) (Life Technologies, Cat. #15140122)

4. Human IFNβ Quantikine ELISA kit (R&D systems)

5. Infinite M1000 plate reader (TECAN)

### Experimental Procedure

Cryopreserved peripheral blood human mononuclear cells (PBMCs) were rapidly thawed and resuspended in Lymphocyte Media and centrifuged at 500×g for 5 minutes. The supernatant was removed and the cell pellets were gently resuspended in cell culture and compound dilution media. Then the cells were plated in a 96-well format at a concentration of 1.5×10⁵ per well. The test compounds, at varying concentrations, or vehicle control (<0.3% DMSO) were mixed with the cells giving a final volume of 150 μl per well. The plates were incubated in a humidified, 5% CO₂ incubator at 37° C. for 5 hours. After incubation, the human IFNβ in the supernatant and the IFNβ standard controls were measured using human IFNβ Quantikine ELISA kit according to the manufacturer's protocol. The absorbance at 450 nm was measured with Infinite M1000 plate reader and corrected by background reading at 540 nm of each well. The concentration of IFNβ secreted was calculated based on the standard curves. The plot of IFNβ concentration versus the log of compound concentration was fit in GraphPad Prism with a 4-parameter concentration response equation to calculate EC₅₀ and Emax (see Table 5).

TABLE 5
IFNβ secretion in human PBMC.

| Example No | EC₅₀ (μM) |
|---|---|
| 1 | 0.22 |
| 2 | 0.026 |
| 9 | 0.14 |
| 10 | 0.05 |
| 11 | 0.19 |
| 12 | 0.058 |

Conclusion: The compounds of the present invention showed significant activity in STING-specific IFNβ generation.

The foregoing embodiments and examples are provided for illustration only and are not intended to limit the scope of the invention. Various changes and modifications to the disclosed embodiments will be apparent to those skilled in the art based on the present disclosure, and such changes and modifications may be made without departure from the spirit and scope of the present invention. All literature cited are incorporated herein by reference in their entireties without admission of them as prior art.

1. A compound of formula (I): 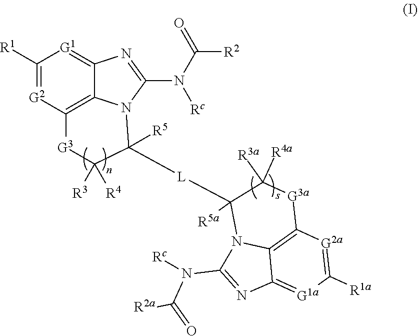
or a tautomer, cis- or trans-isomer, mesomer, racemate, enantiomer, diastereomer, or mixture thereof, or a pharmaceutically acceptable salt, solvate, or prodrug thereof, wherein:
G¹, G², G^{1a} and G^{2a} are identical or different, and each is independently N or CR⁶;
G³ and G^{3a} are identical or different, and each is independently O, NR^{g} or CR⁷R⁸;
L is selected from the group consisting of alkylene, alkenylene, alkynylene, alkylene-Q-alkylene, alkylene-O-alkylene, alkylene-NH-alkylene, alkylene-S(O)m-alkylene, alkylene-C(O)-alkylene, alkylene-C(O)NH-alkylene, alkylene-NHC(O)-alkylene, and alkylene-HNC(O)NH— alkylene, wherein the alkylene, alkenylene and alkynylene at each occurrence is independently unsubstituted or substituted with one or more substituents selected from the group consisting of halogen, alkyl, alkoxy, haloalkyl, amino, nitro, hydroxy, hydroxyalkyl, cyano, cycloalkyl, heterocyclyl, aryl, and heteroaryl;
R^{c} is selected from the group consisting of hydrogen, alkyl, haloalkyl, alkenyl, and alkynyl;
R^{g} each is identical or different, and each is selected from the group consisting of hydrogen, alkyl, cycloalkyl, and alkenyl; wherein the alkyl, cycloalkyl or alkenyl is unsubstituted or substituted with one or more substituents selected from the group consisting of halogen, alkyl, alkoxy, haloalkyl, amino, cyano, hydroxy, hydroxyalkyl, cycloalkyl, heterocyclyl, aryl, and heteroaryl;
R¹ and R^{1a} are identical or different, and each is independently selected from the group consisting of —C(O)NR⁹R¹⁰, —C(O)OR^{m}, hydrogen, halogen, alkyl, alkoxy, haloalkyl, amino, nitro, hydroxy, hydroxyalkyl, cyano, cycloalkyl, heterocyclyl, aryl, and heteroaryl, wherein the alkyl, alkoxy, cycloalkyl, heterocyclyl, aryl, or heteroaryl is unsubstituted or substituted with one or more substituents independently selected from the group consisting of halogen, alkyl, alkoxy, haloalkyl, amino, nitro, hydroxy, hydroxyalkyl, cyano, cycloalkyl, heterocyclyl, aryl, and heteroaryl;
R² and R^{2a} are identical or different, and each is independently selected from the group consisting of alkyl, haloalkyl, cycloalkyl, heterocyclyl, aryl, and heteroaryl; wherein the alkyl, cycloalkyl, heterocyclyl, aryl, or heteroaryl is unsubstituted or substituted with one or more substituents independently selected from the group consisting of halogen, alkyl, alkoxy, haloalkyl, amino, nitro, cyano, hydroxy, hydroxyalkyl, cycloalkyl, heterocyclyl, aryl, and heteroaryl;
R³, R⁴, R^{3a} and R^{4a} are identical or different, and each is independently selected from the group consisting of hydrogen, halogen, alkyl, alkoxy, haloalkyl, amino, nitro, hydroxy, hydroxyalkyl, cyano, cycloalkyl, heterocyclyl, aryl, and heteroaryl;
R⁵ and R^{5a} are identical or different, and each is independently selected from the group consisting of hydrogen, halogen, alkyl, alkoxy, haloalkyl, amino, nitro, hydroxy, hydroxyalkyl, and cyano;
R⁶ is selected from the group consisting of hydrogen, halogen, alkyl, alkoxy, haloalkyl, amino, nitro, hydroxy, hydroxyalkyl, cyano, cycloalkyl, heterocyclyl, aryl, and heteroaryl;
R⁷ and R⁸ are identical or different, and each is independently selected from the group consisting of hydrogen, halogen, alkyl, alkoxy, haloalkyl, amino, nitro, hydroxy, hydroxyalkyl, cyano, cycloalkyl, heterocyclyl, aryl, and heteroaryl;
R⁹ and R¹⁰ are identical or different, and each is independently selected from the group consisting of hydrogen, alkyl, haloalkyl, hydroxy, cycloalkyl, heterocyclyl, aryl, and heteroaryl; wherein the alkyl, cycloalkyl, heterocyclyl, aryl or heteroaryl is unsubstituted or substituted with one or more substituents selected from the group consisting of halogen, alkyl, alkoxy, haloalkyl, amino, nitro, cyano, hydroxy, hydroxyalkyl, cycloalkyl, heterocyclyl, aryl, and heteroaryl;
R^{m} is selected from the group consisting of hydrogen, alkyl, haloalkyl, hydroxyalkyl, cycloalkyl, heterocyclyl, aryl, and heteroaryl;
Q is selected from the group consisting of cycloalkyl, heterocyclyl, aryl, and heteroaryl;
m is 0, 1 or 2;
n is 0, 1, 2 or 3; and
s is 0, 1, 2 or 3.

2. The compound of claim 1, being a compound of formula (IM): 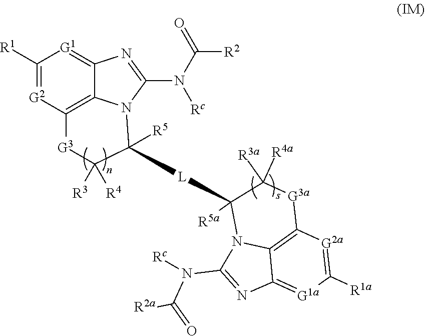
or a tautomer, cis- or trans-isomer, mesomer, racemate, enantiomer, diastereomer, or mixture thereof, or a pharmaceutically acceptable salt, solvate, or prodrug thereof, wherein:
R¹ to R⁵, R^{1a} to R^{5a}, R^{c}, G¹ to G³, G^{1a} to G^{3a}, L, n and s are each as defined in claim 1.

3. The compound of claim 1, or a tautomer, cis- or trans-isomer, mesomer, racemate, enantiomer, diastereomer, or mixture thereof, or a pharmaceutically acceptable salt, solvate, or prodrug thereof, wherein G¹, G², G^{1a} and G^{2a} are identical or different, and each is independently CR⁶; wherein R⁶ at each occurrence is independently as defined in claim 1.

4. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein R² and R^{2a} are identical or different, and each is independently aryl or heteroaryl; wherein the aryl or heteroaryl is unsubstituted or substituted with one or more substituents independently selected from the group consisting of halogen, alkyl, alkoxy, haloalkyl, amino, nitro, cyano, hydroxy, hydroxyalkyl, cycloalkyl, heterocyclyl, aryl, and heteroaryl.

5. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein R⁵ and R^{5a} are each hydrogen.

6. The compound according to claim 1, being a compound of formula (II): 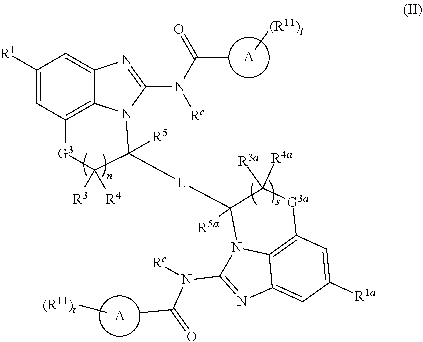
or a tautomer, cis- or trans-isomer, mesomer, racemate, enantiomer, diastereomer, or mixture thereof, or a pharmaceutically acceptable salt, solvate, or prodrug thereof, wherein:
ring A is selected from the group consisting of aryl and heteroaryl;
R¹¹ is each identical or different, and each is independently selected from the group consisting of halogen, alkyl, alkoxy, haloalkyl, amino, nitro, cyano, hydroxy, hydroxyalkyl, cycloalkyl, heterocyclyl, aryl, and heteroaryl;
t is 0, 1, 2, 3 or 4; and
R¹, R^{1a}, R^{c}, R³, R⁴, R^{3a}, R^{4a}, G³, G^{3a}, L, n, and s are each as defined in claim 1.

7. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein R¹ and R^{1a} are identical or different, and each is independently selected from the group consisting of —C(O)NR⁹R¹⁰ and —C(O)OR^{m}, R⁹, R¹⁰ and R^{m} are each as defined in claim 1.

8. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein R^{c} is hydrogen.

9. The compound according to claim 1, being a compound of formula (IG): 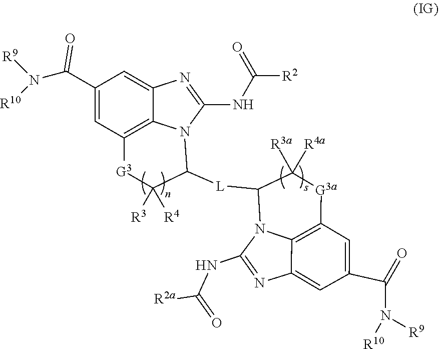
or a tautomer, cis- or trans-isomer, mesomer, racemate, enantiomer, diastereomer, or mixture thereof, or a pharmaceutically acceptable salt, solvate, or prodrug thereof, wherein:
R² to R⁴, R^{2a} to R^{4a}, R⁹, R¹⁰, G³, G^{3a}, L, n and s are each as defined in claim 1.

10. The compound according to claim 1, being a compound of formula (IK): 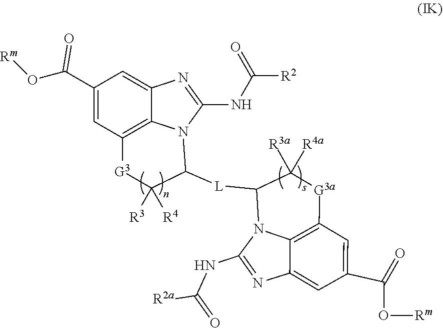
or a tautomer, cis- or trans-isomer, mesomer, racemate, enantiomer, diastereomer, or mixture thereof, or a pharmaceutically acceptable salt, solvate, or prodrug thereof, wherein:
R^{m} is hydrogen or alkyl;
R² to R⁴, R^{2a} to R^{4a}, G³, G^{3a}, L, n and s are each as defined in claim 1.

11. The compound according to claim 1, being a compound of formula (III): 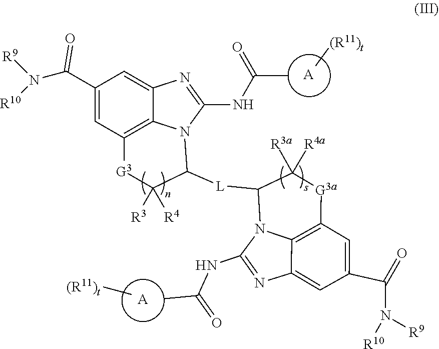
or a tautomer, cis- or trans-isomer, mesomer, racemate, enantiomer, diastereomer, or mixture thereof, or a pharmaceutically acceptable salt, solvate, or prodrug thereof, wherein:
ring A is selected from the group consisting of aryl and heteroaryl;
R⁹ and R¹⁰ are identical or different, and each is independently selected from the group consisting of hydrogen and alkyl;
R¹¹ is each identical or different, and each is independently selected from the group consisting of halogen, alkyl, alkoxy, haloalkyl, amino, nitro, cyano, hydroxy, hydroxyalkyl, cycloalkyl, heterocyclyl, aryl, and heteroaryl;
t is 0, 1, 2, 3 or 4;
R³, R⁴, R^{3a}, R^{4a}, G³, G^{3a}, L, n and s are each as defined in claim 1.

12. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein R³, R⁴, R^{3a} and R^{4a} are hydrogen.

13. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein G³ and G^{3a} are identical or different, and each is independently O or NR^{g}; R^{g} each is identical or different, and each is hydrogen or alkyl, wherein alkyl is unsubstituted or substituted with one or more alkoxy.

14. The compound according to claim 7, or a tautomer, cis- or trans-isomer, mesomer, racemate, enantiomer, diastereomer, or mixture thereof, or a pharmaceutically acceptable salt, solvate, or prodrug thereof, wherein R⁹ and R¹⁰ are each hydrogen.

15. The compound according to claim 11, or a tautomer, cis- or trans-isomer, mesomer, racemate, enantiomer, diastereomer, or mixture thereof, or a pharmaceutically acceptable salt, solvate thereof, wherein A is pyrazole.

16. The compound according to a claim 1, being a compound of formula (IV): 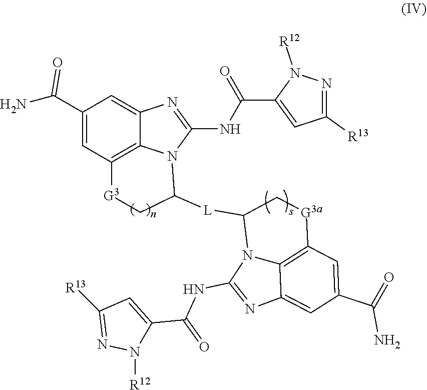
or a tautomer, cis- or trans-isomer, mesomer, racemate, enantiomer, diastereomer, or mixture thereof, or a pharmaceutically acceptable salt, solvate, or prodrug thereof, wherein:
G³ and G^{3a} are identical or different, and each is independently O or NR^{g};
R¹² and R¹³ are identical or different, and each is independently selected from hydrogen and alkyl;
R^{g}, L, n and s are each as defined in claim 1.

17. The compound according to claim 1, being a compound of formula (IVM): 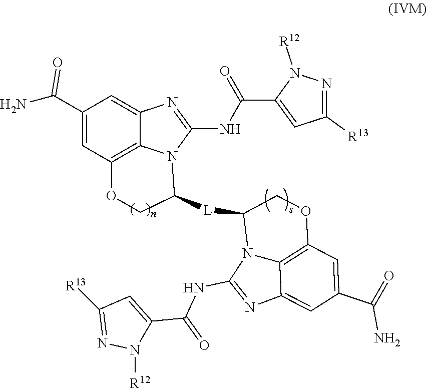
or a tautomer, cis- or trans-isomer, mesomer, racemate, enantiomer, diastereomer, or mixture thereof, or a pharmaceutically acceptable salt, solvate, or prodrug thereof, wherein:
R¹² and R¹³ are identical or different, and each is independently selected from hydrogen and alkyl;
L, n and s are each as defined in claim 1.

18. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein G³ and G^{3a} are identical or different, and each is independently NR^{g}; R^{g} is each as defined in claim 1.

19. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein L is selected from the group consisting of alkylene, alkenylene, alkynylene, alkylene-Q-alkylene, and alkylene-O-alkylene, wherein the alkylene, alkenylene and alkynylene each is unsubstituted or substituted with one or more substituents selected from the group consisting of halogen, alkyl, alkoxy, haloalkyl, amino, nitro, hydroxy, hydroxyalkyl, cyano, cycloalkyl, heterocyclyl, aryl, and heteroaryl; Q is selected from the group consisting of cycloalkyl, heterocyclyl, aryl, and heteroaryl.

20. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein L is selected from the group consisting of —(CH₂)p-, —(CH₂)p¹-(CH═CH)q-(CH₂)p²-, —(CH₂)p¹-C≡C—(CH₂)p²-, —(CH₂)p¹-cyclopropyl-(CH₂)p²-, —(CH₂)p¹-phenyl-(CH₂)p²-, —(CH₂)p¹-O—(CH₂)p²-, and —(CH₂)p¹-(CH(OH))t-(CH₂)p²-; p is an integer of 1 to 6; p¹ is 0, 1, 2 or 3; p² is 0, 1, 2 or 3; q is 0, 1 or 2; and t is 0, 1, 2 or 3.

21. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein L is selected from the group consisting of —CH₂—CH═CH—CH₂—, —CH₂CH₂—, —(CH₂)₃—, —(CH₂)₄—, —CH₂CH(OH)CH(OH)CH₂—, —CH₂—CH═CH—, —CH₂-cyclopropyl-CH₂—, —CH₂-phenyl-CH₂—, —CH₂—C≡C—CH₂—, —CH₂—CH═CH—CH₂CH₂—, and —CH₂—O—CH₂—.

22. The compound according to claim 1, or a tautomer, cis- or trans-isomer, mesomer, racemate, enantiomer, diastereomer, or mixture thereof, or a pharmaceutically acceptable salt, solvate, or prodrug thereof, wherein the compound is selected from the group consisting of:

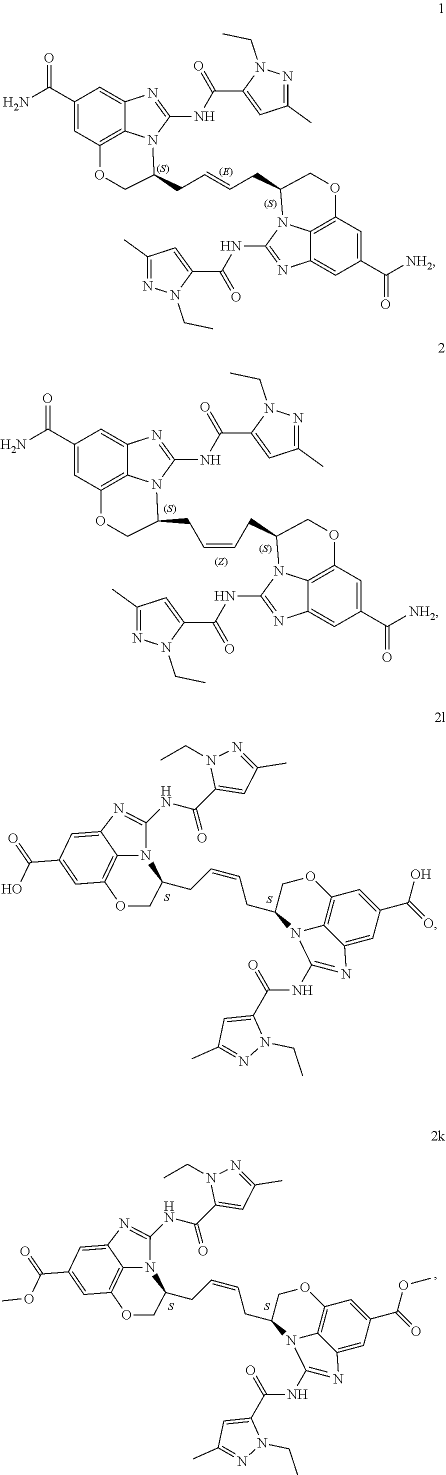

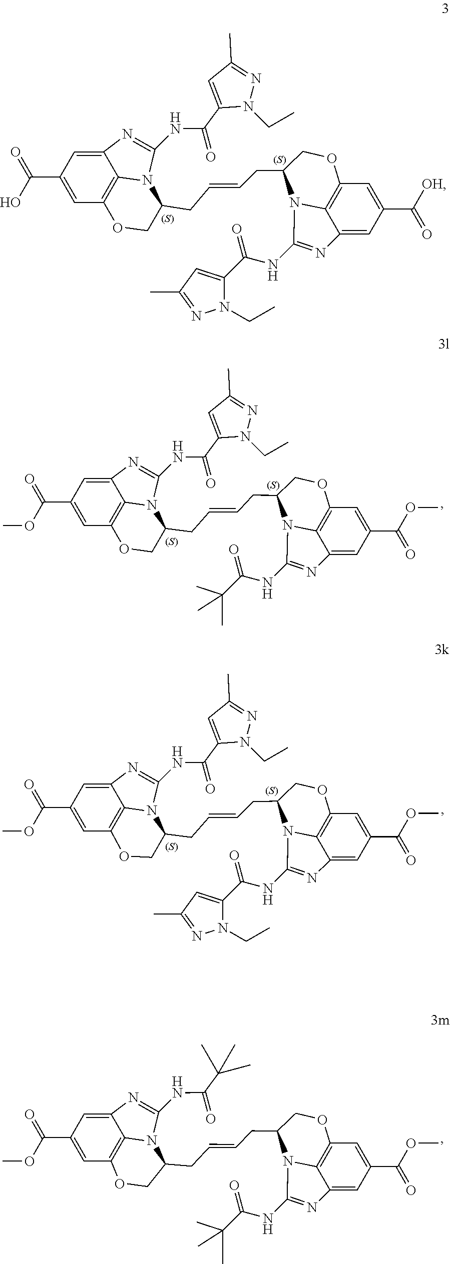

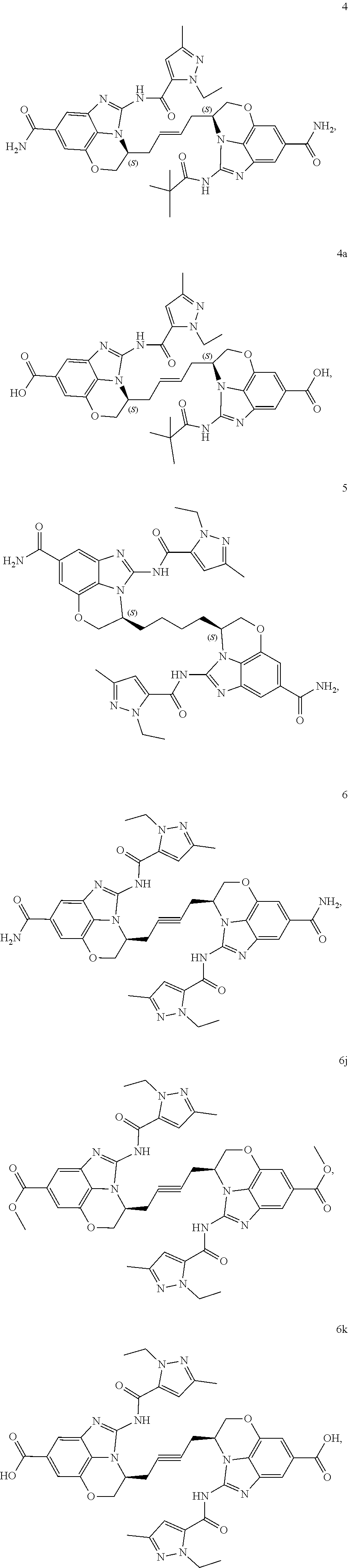

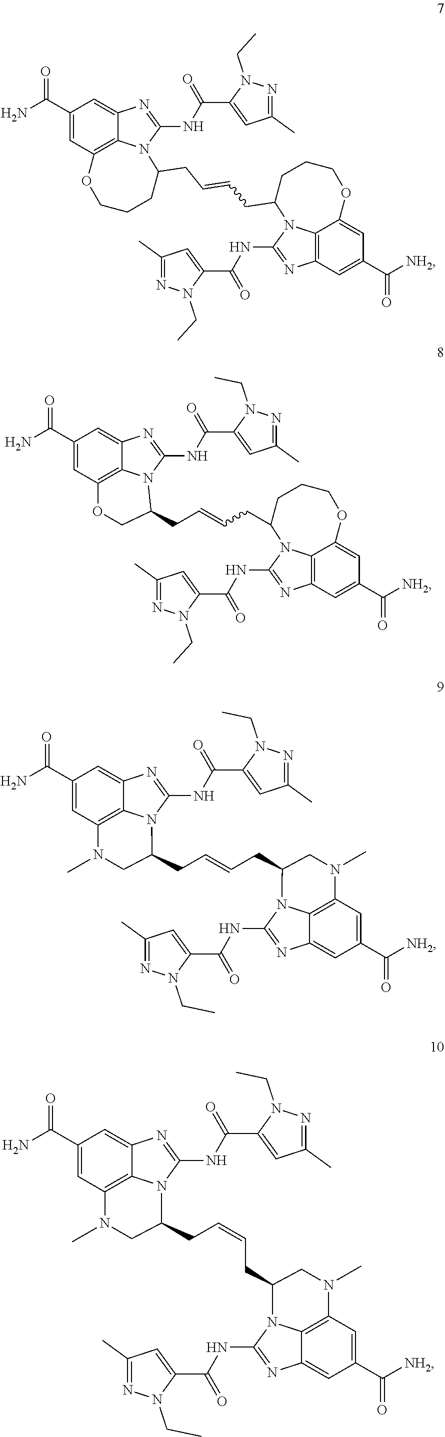 [SMILES: CCn1nc(C)cc1C(=O)Nc1nc2cc(C(N)=O)cc3c2n1C(CC=CCC1CCCOc2cc(C(N)=O)cc4nc(NC(=O)c5cc(C)nn5CC)n1c24)CCCO3 ; CCn1nc(C)cc1C(=O)Nc1nc2cc(C(N)=O)cc3c2n1C(CC=CC[C@H]1COc2cc(C(N)=O)cc4nc(NC(=O)c5cc(C)nn5CC)n1c24)CCCO3 ; CCn1nc(C)cc1C(=O)Nc1nc2cc(C(N)=O)cc3c2n1[C@@H](C/C=C/C[C@H]1CN(C)c2cc(C(N)=O)cc4nc(NC(=O)c5cc(C)nn5CC)n1c24)CN3C ; CCn1nc(C)cc1C(=O)Nc1nc2cc(C(N)=O)cc3c2n1[C@@H](C/C=C\C[C@H]1CN(C)c2cc(C(N)=O)cc4nc(NC(=O)c5cc(C)nn5CC)n1c24)CN3C]

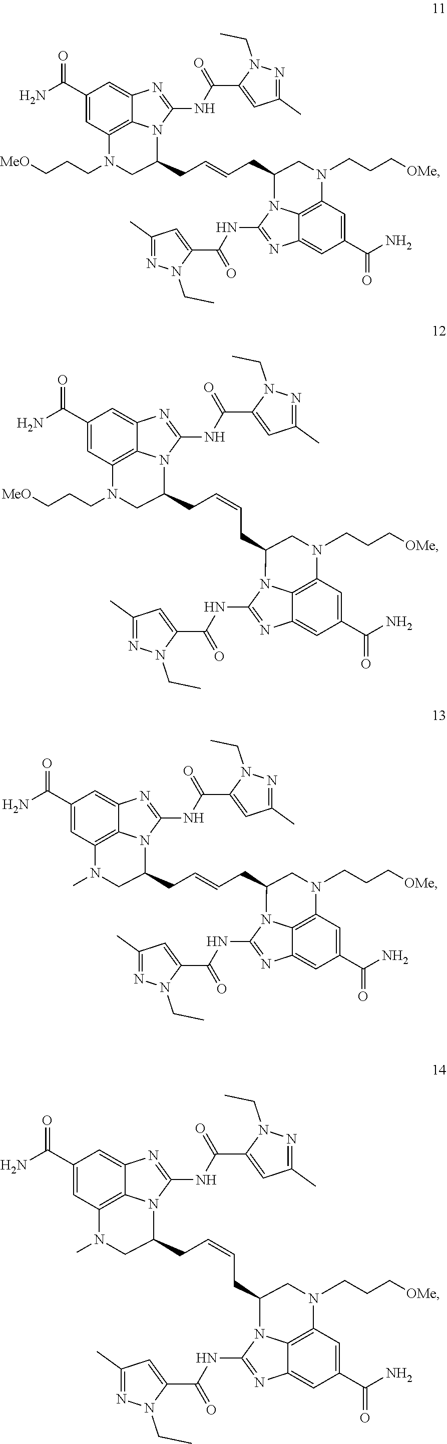 [SMILES: CCn1nc(C)cc1C(=O)Nc1nc2cc(C(N)=O)cc3c2n1[C@@H](C/C=C/C[C@H]1CN(CCCOC)c2cc(C(N)=O)cc4nc(NC(=O)c5cc(C)nn5CC)n1c24)CN3CCCOC ; CCn1nc(C)cc1C(=O)Nc1nc2cc(C(N)=O)cc3c2n1[C@@H](C/C=C\C[C@H]1CN(CCCOC)c2cc(C(N)=O)cc4nc(NC(=O)c5cc(C)nn5CC)n1c24)CN3CCCOC ; CCn1nc(C)cc1C(=O)Nc1nc2cc(C(N)=O)cc3c2n1[C@@H](C/C=C/C[C@H]1CN(CCCOC)c2cc(C(N)=O)cc4nc(NC(=O)c5cc(C)nn5CC)n1c24)CN3C ; CCn1nc(C)cc1C(=O)Nc1nc2cc(C(N)=O)cc3c2n1[C@@H](C/C=C\C[C@H]1CN(CCCOC)c2cc(C(N)=O)cc4nc(NC(=O)c5cc(C)nn5CC)n1c24)CN3C]

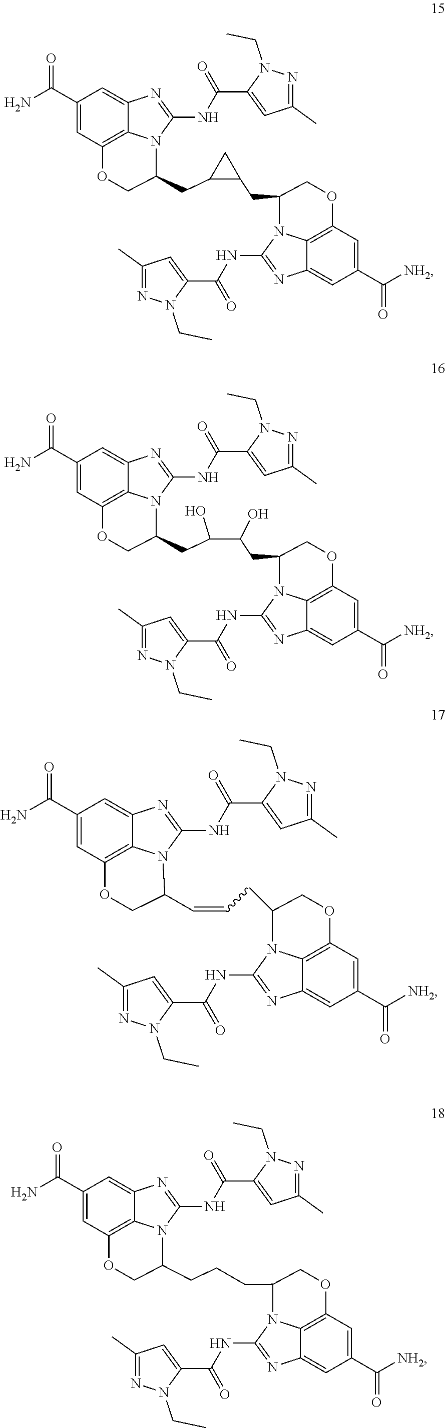 [SMILES: CCn1nc(C)cc1C(=O)Nc1nc2cc(C(N)=O)cc3c2n1[C@@H](CC1CC1C[C@H]1COc2cc(C(N)=O)cc4nc(NC(=O)c5cc(C)nn5CC)n1c24)CO3 ; CCn1nc(C)cc1C(=O)Nc1nc2cc(C(N)=O)cc3c2n1[C@@H](CC(O)C(O)C[C@H]1COc2cc(C(N)=O)cc4nc(NC(=O)c5cc(C)nn5CC)n1c24)CO3 ; CCn1nc(C)cc1C(=O)Nc1nc2cc(C(N)=O)cc3c2n1C(C=CCC1COc2cc(C(N)=O)cc4nc(NC(=O)c5cc(C)nn5CC)n1c24)CO3 ; CCn1nc(C)cc1C(=O)Nc1nc2cc(C(N)=O)cc3c2n1C(CCCC1COc2cc(C(N)=O)cc4nc(NC(=O)c5cc(C)nn5CC)n1c24)CO3]

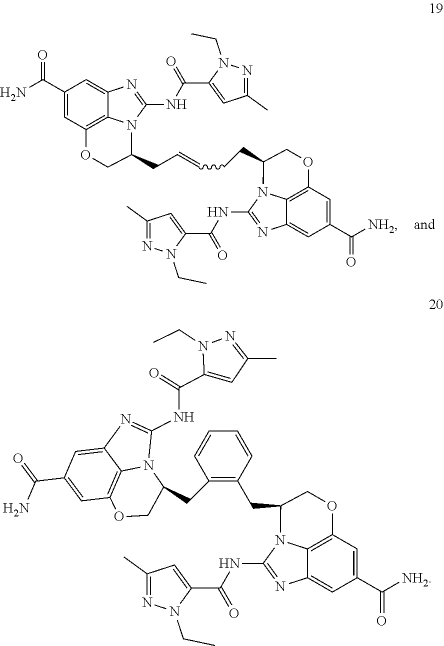 [SMILES: CCn1nc(C)cc1C(=O)Nc1nc2cc(C(N)=O)cc3c2n1[C@@H](CC=CCC[C@H]1COc2cc(C(N)=O)cc4nc(NC(=O)c5cc(C)nn5CC)n1c24)CO3 ; "and" ; CCn1nc(C)cc1C(=O)Nc1nc2cc(C(N)=O)cc3c2n1[C@@H](Cc1ccccc1C[C@H]1COc2cc(C(N)=O)cc4nc(NC(=O)c5cc(C)nn5CC)n1c24)CO3]

23. A compound of formula (IA) or (IB): 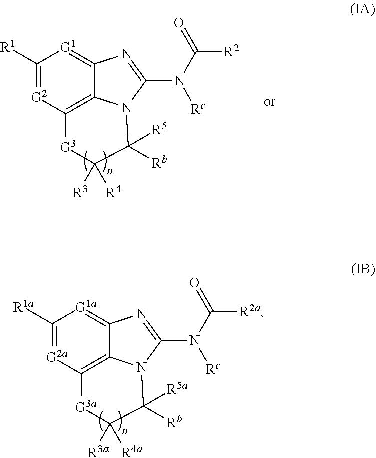
or a tautomer, cis- or trans-isomer, mesomer, racemate, enantiomer, diastereomer, or mixture thereof, or a pharmaceutically acceptable salt, solvate, or prodrug thereof, wherein:
R^{b} is —(CH₂)ₚ₁—CH═CR^{e}R^{f};
R^{d} is —(CH₂)ₚ₂—CH═CR^{e}R^{f};
R^{e} and R^{f} are identical or different, and each is independently selected from the group consisting of hydrogen and alkyl;
p¹ is 0, 1, 2 or 3;
p² is 0, 1, 2 or 3;
R¹ to R⁵, R^{1a} to R^{5a}, R^{c}, G¹ to G³, G^{1a} to G^{3a}, n and s are each as defined in claim 1.

24. The compound of claim 23, or a tautomer, cis- or trans-isomer, mesomer, racemate, enantiomer, diastereomer, or mixture thereof, or a pharmaceutically acceptable salt, solvate, or prodrug thereof, wherein the compound is:

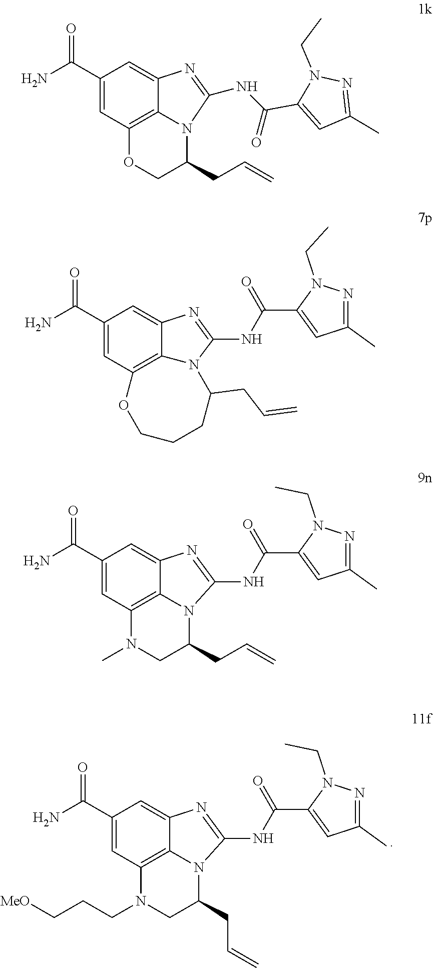 [SMILES: C=CC[C@H]1COc2cc(C(N)=O)cc3nc(NC(=O)c4cc(C)nn4CC)n1c23 ; C=CCC1CCCOc2cc(C(N)=O)cc3nc(NC(=O)c4cc(C)nn4CC)n1c23 ; C=CC[C@H]1CN(C)c2cc(C(N)=O)cc3nc(NC(=O)c4cc(C)nn4CC)n1c23 ; C=CC[C@H]1CN(CCCOC)c2cc(C(N)=O)cc3nc(NC(=O)c4cc(C)nn4CC)n1c23]

25. A process of preparing the compound of formula (I) according to claim 1, or a tautomer, cis- or trans-isomer, mesomer, racemate, enantiomer, diastereomer, or mixture thereof, or a pharmaceutically acceptable salt, solvate, or prodrug thereof, comprising a step of: 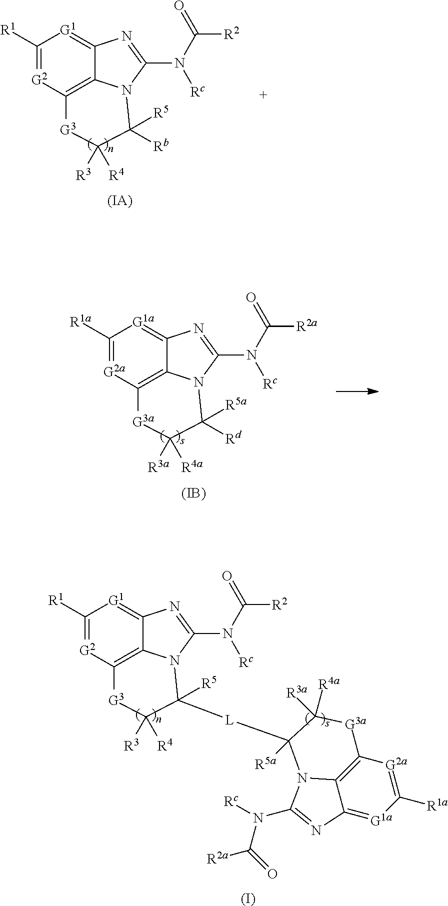
reacting a compound of formula (IA) with a compound of formula (IB) to obtain the compound of formula (I), wherein:
R^{b} is —(CH₂)ₚ₁—CH═CR^{e}R^{f};
R^{d} is —(CH₂)ₚ₂—CH═CR^{e}R^{f};
L is —(CH₂)ₚ₁—CH═CH—(CH₂)ₚ₂—;
R^{e} and R^{f} are identical or different, and each is independently selected from the group consisting of hydrogen and alkyl;
p¹ is 0, 1, 2 or 3;
p² is 0, 1, 2 or 3;
R¹ to R⁵, R^{1a} to R^{5a}, R^{c}, G¹ to G³, G^{1a} to G^{3a}, n and s are each as defined in claim 1.

26. A process of preparing the compound of formula (IG) according to claim 9, or a tautomer, cis- or trans-isomer, mesomer, racemate, enantiomer, diastereomer, or mixture thereof, or a pharmaceutically acceptable salt, solvate, or prodrug thereof, comprising a step of: 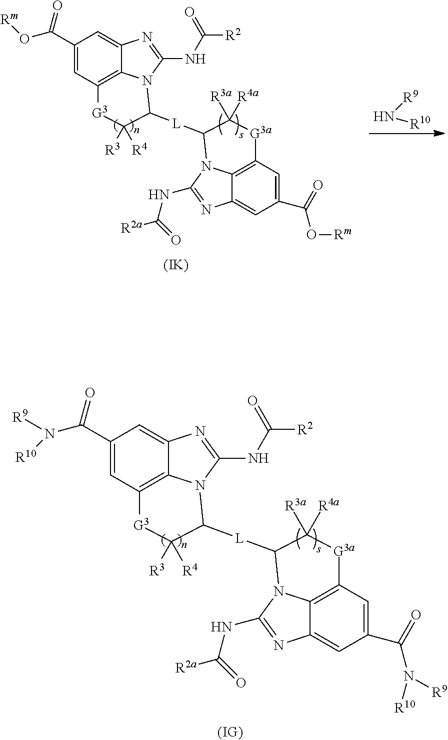
reacting a compound of formula (IK) with a compound of NHR⁹R¹⁰ to obtain the compound of formula (IG), wherein:
R^{m} is hydrogen or alkyl;
R² to R⁴, R^{2a} to R^{4a}, G³, G^{3a}, R⁹, R¹⁰, n and s are each as defined in claim 9.

27. A pharmaceutical composition comprising a therapeutically effective amount of the compound according to claim 1, or a tautomer, cis- or trans-isomer, mesomer, racemate, enantiomer, diastereomer, or mixture thereof, or a pharmaceutically acceptable salt solvate, or prodrug thereof, and a pharmaceutically acceptable carrier.

28-31. (canceled)

32. A method for treating a STING-mediated disease or disorder, comprising a step of administering to a subject in need thereof a therapeutically effective amount of the compound according to claim 1, or a tautomer, cis- or trans-isomer, mesomer, racemate, enantiomer, diastereomer, or mixture thereof, or a pharmaceutically acceptable salt solvate, or prodrug thereof.

33. The method according to claim 32, wherein the disease or disorder is selected from a cancer, a pre-cancerous syndrome and viral infections, preferably a cancer and a pre-cancerous syndrome.
